SEQUENCE LISTING (165 sequences)

SEQ ID NO: 1 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 16 aa, synthetic construct
KRPAATKKAGQAKKKK

SEQ ID NO: 3 - RNA, 20 nt, Acidaminococcus sp.
  source 1..20: mol_type = genomic RNA
taatttctactcttgtagat

SEQ ID NO: 4 - RNA, 40 nt, synthetic construct
  source 1..40: mol_type = other RNA
  misc_difference 21: note = a, c, u, g, unknown or other
  misc_difference 22: note = a, c, u, g, unknown or other
  misc_difference 23: note = a, c, u, g, unknown or other
  misc_difference 24: note = a, c, u, g, unknown or other
  misc_difference 25: note = a, c, u, g, unknown or other
  misc_difference 26: note = a, c, u, g, unknown or other
  misc_difference 27: note = a, c, u, g, unknown or other
  misc_difference 28: note = a, c, u, g, unknown or other
  misc_difference 29: note = a, c, u, g, unknown or other
  misc_difference 30: note = a, c, u, g, unknown or other
  misc_difference 31: note = a, c, u, g, unknown or other
  misc_difference 32: note = a, c, u, g, unknown or other
  misc_difference 33: note = a, c, u, g, unknown or other
  misc_difference 34: note = a, c, u, g, unknown or other
  misc_difference 35: note = a, c, u, g, unknown or other
  misc_difference 36: note = a, c, u, g, unknown or other
  misc_difference 37: note = a, c, u, g, unknown or other
  misc_difference 38: note = a, c, u, g, unknown or other
  misc_difference 39: note = a, c, u, g, unknown or other
  misc_difference 40: note = a, c, u, g, unknown or other
taatttctactcttgtagatnnnnnnnnnnnnnnnnnnnn

SEQ ID NO: 5 - DNA, 24 bp, Homo sapiens
  source 1..24: mol_type = genomic DNA
tttgttgttcctttagtaattgca

SEQ ID NO: 6 - DNA, 24 bp, Homo sapiens
  source 1..24: mol_type = genomic DNA
tttagtaattgcatccagagtgga

SEQ ID NO: 7 - DNA, 24 bp, Homo sapiens
  source 1..24: mol_type = genomic DNA
tttggctctgaaggtgcagaatca

SEQ ID NO: 8 - DNA, 24 bp, Homo sapiens
  source 1..24: mol_type = genomic DNA
tttcatcatttgattctgcacctt

SEQ ID NO: 9 - DNA, 24 bp, Homo sapiens
  source 1..24: mol_type = genomic DNA
tttgattctgcaccttcagagcca

SEQ ID NO: 10 - DNA, 24 bp, Homo sapiens
  source 1..24: mol_type = genomic DNA
tttgacatcatccaacatagcaaa

SEQ ID NO: 11 - DNA, 24 bp, Homo sapiens
  source 1..24: mol_type = genomic DNA
tttgctatgttggatgatgtcaaa

SEQ ID NO: 12 - DNA, 24 bp, Homo sapiens
  source 1..24: mol_type = genomic DNA
tttagcgaatggcctcctgcagct

SEQ ID NO: 13 - DNA, 24 bp, Homo sapiens
  source 1..24: mol_type = genomic DNA
tttaagtccatgacccagctgcag

SEQ ID NO: 14 - DNA, 24 bp, Homo sapiens
  source 1..24: mol_type = genomic DNA
tttgtcccttagtcttatggacaa

SEQ ID NO: 15 - DNA, 24 bp, Homo sapiens
  source 1..24: mol_type = genomic DNA
tttgtccataagactaagggacaa

SEQ ID NO: 16 - DNA, 24 bp, Homo sapiens
  source 1..24: mol_type = genomic DNA
tttcagaagctcaacatatttgat

SEQ ID NO: 17 - DNA, 24 bp, Homo sapiens
  source 1..24: mol_type = genomic DNA
tttgatcagtctttttatgaccta

SEQ ID NO: 18 - DNA, 24 bp, Homo sapiens
  source 1..24: mol_type = genomic DNA
tttcattggttcgaagtgataggt

SEQ ID NO: 19 - DNA, 24 bp, Homo sapiens
  source 1..24: mol_type = genomic DNA
tttgatttcattggttcgaagtga

SEQ ID NO: 20 - DNA, 24 bp, Homo sapiens
  source 1..24: mol_type = genomic DNA
tttatgacctatcacttcgaacca

SEQ ID NO: 21 - DNA, 24 bp, Homo sapiens
  source 1..24: mol_type = genomic DNA
tttcctcttctttgatttcattgg

SEQ ID NO: 22 - DNA, 24 bp, Homo sapiens
  source 1..24: mol_type = genomic DNA
tttaacttgtagtgtagatgtagt

SEQ ID NO: 23 - DNA, 24 bp, Homo sapiens
  source 1..24: mol_type = genomic DNA
tttgagttcagttctactgacatg

SEQ ID NO: 24 - DNA, 24 bp, Homo sapiens
  source 1..24: mol_type = genomic DNA
tttggaggagcagctaaccaactt

SEQ ID NO: 25 - DNA, 24 bp, Homo sapiens
  source 1..24: mol_type = genomic DNA
tttgagtgatgttacttctgggtg

SEQ ID NO: 26 - DNA, 24 bp, Homo sapiens
  source 1..24: mol_type = genomic DNA
tttctacttactttgagtgatgtt

SEQ ID NO: 27 - DNA, 24 bp, Homo sapiens
  source 1..24: mol_type = genomic DNA
tttgtagaacagcaagacaacagc

SEQ ID NO: 28 - DNA, 24 bp, Homo sapiens
  source 1..24: mol_type = genomic DNA
tttatactgttcttccacactctg

SEQ ID NO: 29 - DNA, 24 bp, Homo sapiens
  source 1..24: mol_type = genomic DNA
tttatctgcatgtgctgttgactt

SEQ ID NO: 30 - DNA, 24 bp, Homo sapiens
  source 1..24: mol_type = genomic DNA
tttcttttatctgcatgtgctgtt

SEQ ID NO: 31 - DNA, 24 bp, Homo sapiens
  source 1..24: mol_type = genomic DNA
tttctatttcttttatctgcatgt

SEQ ID NO: 32 - DNA, 24 bp, Homo sapiens
  source 1..24: mol_type = genomic DNA
tttgtattcttttatcagctcaga

SEQ ID NO: 33 - DNA, 24 bp, Homo sapiens
  source 1..24: mol_type = genomic DNA
tttatcagctcagaaagactggta

SEQ ID NO: 34 - DNA, 24 bp, Homo sapiens
  source 1..24: mol_type = genomic DNA
tttctgagggttcttgaataccag

SEQ ID NO: 35 - DNA, 24 bp, Homo sapiens
  source 1..24: mol_type = genomic DNA
tttagaagaaagagaattttctga

SEQ ID NO: 36 - DNA, 24 bp, Homo sapiens
  source 1..24: mol_type = genomic DNA
tttcttctaaatcaagagcaccaa

SEQ ID NO: 37 - DNA, 24 bp, Homo sapiens
  source 1..24: mol_type = genomic DNA
tttcgttcagttgaagagggggag

SEQ ID NO: 38 - DNA, 24 bp, Homo sapiens
  source 1..24: mol_type = genomic DNA
tttctgtttcgttcagttgaagag

SEQ ID NO: 39 - DNA, 24 bp, Homo sapiens
  source 1..24: mol_type = genomic DNA
tttataacagaggcgaacatacaa

SEQ ID NO: 40 - DNA, 24 bp, Homo sapiens
  source 1..24: mol_type = genomic DNA
tttaatagtgtacacgccacttgt

SEQ ID NO: 41 - DNA, 24 bp, Homo sapiens
  source 1..24: mol_type = genomic DNA
tttcttggtttaatagtgtacacg

SEQ ID NO: 42 - DNA, 24 bp, Homo sapiens
  source 1..24: mol_type = genomic DNA
tttacctgattgggtatcacagta

SEQ ID NO: 43 - DNA, 24 bp, Homo sapiens
  source 1..24: mol_type = genomic DNA
tttaatgtctactgtgatacccaa

SEQ ID NO: 44 - DNA, 24 bp, Homo sapiens
  source 1..24: mol_type = genomic DNA
tttccatgggtttacctgattggg

SEQ ID NO: 45 - DNA, 24 bp, Homo sapiens
  source 1..24: mol_type = genomic DNA
tttccggtgttgaattaatgtcca

SEQ ID NO: 46 - DNA, 24 bp, Homo sapiens
  source 1..24: mol_type = genomic DNA
tttcgttgaagtcctgtgagccat

SEQ ID NO: 47 - DNA, 24 bp, Homo sapiens
  source 1..24: mol_type = genomic DNA
tttcccatgtttcgttgaagtcct

SEQ ID NO: 48 - DNA, 24 bp, Homo sapiens
  source 1..24: mol_type = genomic DNA
tttcgtagttttcccatgtttcgt

SEQ ID NO: 49 - DNA, 24 bp, Homo sapiens
  source 1..24: mol_type = genomic DNA
tttaccatcgagcctcccaaagcc

SEQ ID NO: 50 - DNA, 24 bp, Homo sapiens
  source 1..24: mol_type = genomic DNA
tttgggaggctcgatggtaaagtg

SEQ ID NO: 51 - DNA, 24 bp, Homo sapiens
  source 1..24: mol_type = genomic DNA
tttctttaggagaattttggttgg

SEQ ID NO: 52 - DNA, 24 bp, Homo sapiens
  source 1..24: mol_type = genomic DNA
tttaggagaattttggttgggcct

SEQ ID NO: 53 - DNA, 24 bp, Homo sapiens
  source 1..24: mol_type = genomic DNA
tttggttgggcctagagaagatct

SEQ ID NO: 54 - DNA, 24 bp, Homo sapiens
  source 1..24: mol_type = genomic DNA
tttccagtcttgtagctcgagtcg

SEQ ID NO: 55 - DNA, 24 bp, Homo sapiens
  source 1..24: mol_type = genomic DNA
tttacgactcgagctacaagactg

SEQ ID NO: 56 - DNA, 24 bp, Homo sapiens
  source 1..24: mol_type = genomic DNA
tttcgtgactgcccaggtgaaagg

SEQ ID NO: 57 - DNA, 24 bp, Homo sapiens
  source 1..24: mol_type = genomic DNA
tttcacctgggcagtcacgaaacc

SEQ ID NO: 58 - DNA, 24 bp, Homo sapiens
  source 1..24: mol_type = genomic DNA
tttgctctgtgattccatgtagaa

SEQ ID NO: 59 - DNA, 24 bp, Homo sapiens
  source 1..24: mol_type = genomic DNA
tttctacatggaatcacagagcaa

SEQ ID NO: 60 - DNA, 24 bp, Homo sapiens
  source 1..24: mol_type = genomic DNA
tttctggacagtagagctgtccct

SEQ ID NO: 61 - DNA, 24 bp, Homo sapiens
  source 1..24: mol_type = genomic DNA
tttctccacatatgtcattccacc

SEQ ID NO: 62 - DNA, 24 bp, Homo sapiens
  source 1..24: mol_type = genomic DNA
tttaggtggctggtggtggaatga

SEQ ID NO: 63 - DNA, 24 bp, Homo sapiens
  source 1..24: mol_type = genomic DNA
tttaggttgttttctccacatatg

SEQ ID NO: 64 - DNA, 24 bp, Homo sapiens
  source 1..24: mol_type = genomic DNA
tttccatttaggttgttttctcca

SEQ ID NO: 65 - DNA, 24 bp, Homo sapiens
  source 1..24: mol_type = genomic DNA
tttgttgtattttccatttaggtt

SEQ ID NO: 66 - DNA, 24 bp, Homo sapiens
  source 1..24: mol_type = genomic DNA
tttggttctgggtttgttgtattt

SEQ ID NO: 67 - DNA, 24 bp, Homo sapiens
  source 1..24: mol_type = genomic DNA
tttctgctctgaggtctccagtag

SEQ ID NO: 68 - DNA, 24 bp, Homo sapiens
  source 1..24: mol_type = genomic DNA
tttgatagcatagagctttctgct

SEQ ID NO: 69 - DNA, 24 bp, Homo sapiens
  source 1..24: mol_type = genomic DNA
tttggatgatttgatagcatagag

SEQ ID NO: 70 - DNA, 24 bp, Homo sapiens
  source 1..24: mol_type = genomic DNA
tttgttcaatctgtagcctctggg

SEQ ID NO: 71 - DNA, 24 bp, Homo sapiens
  source 1..24: mol_type = genomic DNA
tttatgacctcttccctggcttct

SEQ ID NO: 72 - DNA, 24 bp, Homo sapiens
  source 1..24: mol_type = genomic DNA
tttgtcttcgcccagagcatccca

SEQ ID NO: 73 - DNA, 24 bp, Homo sapiens
  source 1..24: mol_type = genomic DNA
tttgcaggcctggagtttattcgg

SEQ ID NO: 74 - DNA, 24 bp, Homo sapiens
  source 1..24: mol_type = genomic DNA
tttattcggaagagtcagctaatc

SEQ ID NO: 75 - DNA, 24 bp, Homo sapiens
  source 1..24: mol_type = genomic DNA
tttaccatgccaggtcatcacagt

SEQ ID NO: 76 - DNA, 24 bp, Homo sapiens
  source 1..24: mol_type = genomic DNA
tttggggactaattttggacgctg

SEQ ID NO: 77 - DNA, 24 bp, Homo sapiens
  source 1..24: mol_type = genomic DNA
tttggacgctgtgtggatctcttt

SEQ ID NO: 78 - DNA, 24 bp, Homo sapiens
  source 1..24: mol_type = genomic DNA
tttgcccccgggaaggacatcatc

SEQ ID NO: 79 - DNA, 24 bp, Homo sapiens
  source 1..24: mol_type = genomic DNA
tttggtagagaagtggatcagcct

SEQ ID NO: 80 - DNA, 24 bp, Homo sapiens
  source 1..24: mol_type = genomic DNA
tttctccaactctcaggccatagg

SEQ ID NO: 81 - DNA, 24 bp, Homo sapiens
  source 1..24: mol_type = genomic DNA
tttgggggtgagggtgtctatgcc

SEQ ID NO: 82 - DNA, 24 bp, Homo sapiens
  source 1..24: mol_type = genomic DNA
tttgcattccagccctggggcatg

SEQ ID NO: 83 - DNA, 24 bp, Homo sapiens
  source 1..24: mol_type = genomic DNA
tttgctgaaggccggctccggcag

SEQ ID NO: 84 - DNA, 24 bp, Homo sapiens
  source 1..24: mol_type = genomic DNA
tttgcctcgctggactggtatttg

SEQ ID NO: 85 - DNA, 24 bp, Homo sapiens
  source 1..24: mol_type = genomic DNA
tttgtgtctgaagctggccccgcg

SEQ ID NO: 86 - DNA, 24 bp, Homo sapiens
  source 1..24: mol_type = genomic DNA
tttggattctccagcaccctgggc

SEQ ID NO: 87 - DNA, 24 bp, Homo sapiens
  source 1..24: mol_type = genomic DNA
tttgaccatcagaggacatttgga

SEQ ID NO: 88 - DNA, 24 bp, Homo sapiens
  source 1..24: mol_type = genomic DNA
tttacagccacgtctacagcaggg

SEQ ID NO: 89 - DNA, 24 bp, Homo sapiens
  source 1..24: mol_type = genomic DNA
tttgaacacttttacagccacgtc

SEQ ID NO: 90 - DNA, 24 bp, Homo sapiens
  source 1..24: mol_type = genomic DNA
tttctacaagcttacccagaggca

SEQ ID NO: 91 - DNA, 24 bp, Homo sapiens
  source 1..24: mol_type = genomic DNA
tttgtagaaggagtgtacagagta

SEQ ID NO: 92 - DNA, 24 bp, Homo sapiens
  source 1..24: mol_type = genomic DNA
tttggtgtccagttctactctgta

SEQ ID NO: 93 - DNA, 24 bp, Homo sapiens
  source 1..24: mol_type = genomic DNA
tttccccgttccatgaattcgcgg

SEQ ID NO: 94 - DNA, 24 bp, Homo sapiens
  source 1..24: mol_type = genomic DNA
tttcacagccaacgactctggcca

SEQ ID NO: 95 - DNA, 40 bp, synthetic construct
  source 1..40: mol_type = other DNA
  misc_feature 1..20: note = RNA
  misc_feature 21: note = DNA
  misc_feature 22..40: note = RNA
taatttctactcttgtagatttcggaagagtcagctaatc

SEQ ID NO: 96 - DNA, 40 bp, synthetic construct; identical to SEQ ID NO: 95
  source 1..40: mol_type = other DNA
  misc_feature 1..27: note = RNA
  misc_feature 28: note = DNA
  misc_feature 29..40: note = RNA

SEQ ID NO: 97 - DNA, 40 bp, synthetic construct; identical to SEQ ID NO: 95
  source 1..40: mol_type = other DNA
  misc_feature 1..28: note = RNA
  misc_feature 29: note = DNA
  misc_feature 30..40: note = RNA

SEQ ID NO: 98 - DNA, 40 bp, synthetic construct; identical to SEQ ID NO: 95
  source 1..40: mol_type = other DNA
  misc_feature 1..29: note = RNA
  misc_feature 30: note = DNA
  misc_feature 31..40: note = RNA

SEQ ID NO: 99 - DNA, 40 bp, synthetic construct; identical to SEQ ID NO: 95
  source 1..40: mol_type = other DNA
  misc_feature 1..30: note = RNA
  misc_feature 31: note = DNA
  misc_feature 32..40: note = RNA

SEQ ID NO: 100 - DNA, 40 bp, synthetic construct; identical to SEQ ID NO: 95
  source 1..40: mol_type = other DNA
  misc_feature 1..31: note = RNA
  misc_feature 32: note = DNA
  misc_feature 33..40: note = RNA

SEQ ID NO: 101 - DNA, 40 bp, synthetic construct; identical to SEQ ID NO: 95
  source 1..40: mol_type = other DNA
  misc_feature 1..33: note = RNA
  misc_feature 34: note = DNA
  misc_feature 35..40: note = RNA

SEQ ID NO: 102 - DNA, 40 bp, synthetic construct; identical to SEQ ID NO: 95
  source 1..40: mol_type = other DNA
  misc_feature 1..34: note = RNA
  misc_feature 35: note = DNA
  misc_feature 36..40: note = RNA

SEQ ID NO: 103 - DNA, 40 bp, synthetic construct; identical to SEQ ID NO: 95
  source 1..40: mol_type = other DNA
  misc_feature 1..36: note = RNA
  misc_feature 37: note = DNA
  misc_feature 38..40: note = RNA

SEQ ID NO: 104 - DNA, 40 bp, synthetic construct; identical to SEQ ID NO: 95
  source 1..40: mol_type = other DNA
  misc_feature 1..37: note = RNA
  misc_feature 38: note = DNA
  misc_feature 39..40: note = RNA

SEQ ID NO: 105 - DNA, 40 bp, synthetic construct; identical to SEQ ID NO: 95
  source 1..40: mol_type = other DNA
  misc_feature 1..38: note = RNA
  misc_feature 39: note = DNA
  misc_feature 40: note = RNA

SEQ ID NO: 106 - DNA, 40 bp, synthetic construct; identical to SEQ ID NO: 95
  source 1..40: mol_type = other DNA
  misc_feature 1..39: note = RNA
  misc_feature 40: note = DNA

SEQ ID NO: 107 - DNA, 40 bp, synthetic construct
  source 1..40: mol_type = other DNA
  misc_feature 1..20: note = RNA
  misc_feature 21: note = DNA
  misc_feature 22..40: note = RNA
taatttctactcttgtagatgtgtccagttctactctgta

SEQ ID NO: 108 - DNA, 40 bp, synthetic construct; identical to SEQ ID NO: 107
  source 1..40: mol_type = other DNA
  misc_feature 1..27: note = RNA
  misc_feature 28: note = DNA
  misc_feature 29..40: note = RNA

SEQ ID NO: 109 - DNA, 40 bp, synthetic construct; identical to SEQ ID NO: 107
  source 1..40: mol_type = other DNA
  misc_feature 1..28: note = RNA
  misc_feature 29: note = DNA
  misc_feature 30..40: note = RNA

SEQ ID NO: 110 - DNA, 40 bp, synthetic construct; identical to SEQ ID NO: 107
  source 1..40: mol_type = other DNA
  misc_feature 1..29: note = RNA
  misc_feature 30: note = DNA
  misc_feature 31..40: note = RNA

SEQ ID NO: 111 - DNA, 40 bp, synthetic construct; identical to SEQ ID NO: 107
  source 1..40: mol_type = other DNA
  misc_feature 1..30: note = RNA
  misc_feature 31: note = DNA
  misc_feature 32..40: note = RNA

SEQ ID NO: 112 - DNA, 40 bp, synthetic construct; identical to SEQ ID NO: 107
  source 1..40: mol_type = other DNA
  misc_feature 1..31: note = RNA
  misc_feature 32: note = DNA
  misc_feature 33..40: note = RNA

SEQ ID NO: 113 - DNA, 40 bp, synthetic construct; identical to SEQ ID NO: 107
  source 1..40: mol_type = other DNA
  misc_feature 1..33: note = RNA
  misc_feature 34: note = DNA
  misc_feature 35..40: note = RNA

SEQ ID NO: 114 - DNA, 40 bp, synthetic construct; identical to SEQ ID NO: 107
  source 1..40: mol_type = other DNA
  misc_feature 1..34: note = RNA
  misc_feature 35: note = DNA
  misc_feature 36..40: note = RNA

SEQ ID NO: 115 - DNA, 40 bp, synthetic construct; identical to SEQ ID NO: 107
  source 1..40: mol_type = other DNA
  misc_feature 1..36: note = RNA
  misc_feature 37: note = DNA
  misc_feature 38..40: note = RNA

SEQ ID NO: 116 - DNA, 40 bp, synthetic construct; identical to SEQ ID NO: 107
  source 1..40: mol_type = other DNA
  misc_feature 1..37: note = RNA
  misc_feature 38: note = DNA
  misc_feature 39..40: note = RNA

SEQ ID NO: 117 - DNA, 40 bp, synthetic construct; identical to SEQ ID NO: 107
  source 1..40: mol_type = other DNA
  misc_feature 1..38: note = RNA
  misc_feature 39: note = DNA
  misc_feature 40: note = RNA

SEQ ID NO: 118 - DNA, 40 bp, synthetic construct; identical to SEQ ID NO: 107
  source 1..40: mol_type = other DNA
  misc_feature 1..39: note = RNA
  misc_feature 40: note = DNA

SEQ ID NO: 119 - DNA, 40 bp, synthetic construct
  source 1..40: mol_type = other DNA
  misc_feature 1..20: note = RNA
  misc_feature 21: note = DNA
  misc_feature 22..40: note = RNA
taatttctactcttgtagatacttgtagtgtagatgtagt

SEQ ID NO: 120 - DNA, 40 bp, synthetic construct; identical to SEQ ID NO: 119
  source 1..40: mol_type = other DNA
  misc_feature 1..27: note = RNA
  misc_feature 28: note = DNA
  misc_feature 29..40: note = RNA

SEQ ID NO: 121 - DNA, 40 bp, synthetic construct; identical to SEQ ID NO: 119
  source 1..40: mol_type = other DNA
  misc_feature 1..28: note = RNA
  misc_feature 29: note = DNA
  misc_feature 30..40: note = RNA

SEQ ID NO: 122 - DNA, 40 bp, synthetic construct; identical to SEQ ID NO: 119
  source 1..40: mol_type = other DNA
  misc_feature 1..29: note = RNA
  misc_feature 30: note = DNA
  misc_feature 31..40: note = RNA

SEQ ID NO: 123 - DNA, 40 bp, synthetic construct; identical to SEQ ID NO: 119
  source 1..40: mol_type = other DNA
  misc_feature 1..30: note = RNA
  misc_feature 31: note = DNA
  misc_feature 32..40: note = RNA

SEQ ID NO: 124 - DNA, 40 bp, synthetic construct; identical to SEQ ID NO: 119
  source 1..40: mol_type = other DNA
  misc_feature 1..31: note = RNA
  misc_feature 32: note = DNA
  misc_feature 33..40: note = RNA

SEQ ID NO: 125 - DNA, 40 bp, synthetic construct; identical to SEQ ID NO: 119
  source 1..40: mol_type = other DNA
  misc_feature 1..33: note = RNA
  misc_feature 34: note = DNA
  misc_feature 35..40: note = RNA

SEQ ID NO: 126 - DNA, 40 bp, synthetic construct; identical to SEQ ID NO: 119
  source 1..40: mol_type = other DNA
  misc_feature 1..34: note = RNA
  misc_feature 35: note = DNA
  misc_feature 36..40: note = RNA

SEQ ID NO: 127 - DNA, 40 bp, synthetic construct; identical to SEQ ID NO: 119
  source 1..40: mol_type = other DNA
  misc_feature 1..36: note = RNA
  misc_feature 37: note = DNA
  misc_feature 38..40: note = RNA

SEQ ID NO: 128 - DNA, 40 bp, synthetic construct; identical to SEQ ID NO: 119
  source 1..40: mol_type = other DNA
  misc_feature 1..37: note = RNA
  misc_feature 38: note = DNA
  misc_feature 39..40: note = RNA

SEQ ID NO: 129 - DNA, 40 bp, synthetic construct; identical to SEQ ID NO: 119
  source 1..40: mol_type = other DNA
  misc_feature 1..38: note = RNA
  misc_feature 39: note = DNA
  misc_feature 40: note = RNA

SEQ ID NO: 130 - DNA, 40 bp, synthetic construct; identical to SEQ ID NO: 119
  source 1..40: mol_type = other DNA
  misc_feature 1..39: note = RNA
  misc_feature 40: note = DNA

SEQ ID NO: 131 - DNA, 40 bp, synthetic construct; identical to SEQ ID NO: 95
  source 1..40: mol_type = other DNA
  misc_feature 1..20: note = RNA
  misc_feature 21: note = DNA
  misc_feature 22..39: note = RNA
  misc_feature 40: note = DNA

SEQ ID NO: 132 - DNA, 40 bp, synthetic construct; identical to SEQ ID NO: 95
  source 1..40: mol_type = other DNA
  misc_feature 1..20: note = RNA
  misc_feature 21: note = DNA
  misc_feature 22..36: note = RNA
  misc_feature 37: note = DNA
  misc_feature 38..39: note = RNA
  misc_feature 40: note = DNA

SEQ ID NO: 133 - DNA, 40 bp, synthetic construct; identical to SEQ ID NO: 95
  source 1..40: mol_type = other DNA
  misc_feature 1..20: note = RNA
  misc_feature 21: note = DNA
  misc_feature 22..36: note = RNA
  misc_feature 37: note = DNA
  misc_feature 38..40: note = RNA

SEQ ID NO: 134 - DNA, 40 bp, synthetic construct; identical to SEQ ID NO: 95
  source 1..40: mol_type = other DNA
  misc_feature 1..36: note = RNA
  misc_feature 37: note = DNA
  misc_feature 38..39: note = RNA
  misc_feature 40: note = DNA

SEQ ID NO: 135 - DNA, 40 bp, synthetic construct; identical to SEQ ID NO: 95
  source 1..40: mol_type = other DNA
  misc_feature 1..26: note = RNA
  misc_feature 27: note = DNA
  misc_feature 28..35: note = RNA
  misc_feature 36: note = DNA
  misc_feature 37..40: note = RNA

SEQ ID NO: 136 - DNA, 40 bp, synthetic construct; identical to SEQ ID NO: 95
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 137 - DNA, 40 bp, synthetic construct; identical to SEQ ID NO: 107
  source 1..40: mol_type = other DNA
  misc_feature 1..20: note = RNA
  misc_feature 21: note = DNA
  misc_feature 22..28: note = RNA
  misc_feature 29: note = DNA
  misc_feature 30..39: note = RNA
  misc_feature 40: note = DNA

SEQ ID NO: 138 - DNA, 40 bp, synthetic construct; identical to SEQ ID NO: 107
  source 1..40: mol_type = other DNA
  misc_feature 1..28: note = RNA
  misc_feature 29: note = DNA
  misc_feature 30..38: note = RNA
  misc_feature 39..40: note = DNA

SEQ ID NO: 139 - DNA, 40 bp, synthetic construct; identical to SEQ ID NO: 107
  source 1..40: mol_type = other DNA
  misc_feature 1..20: note = RNA
  misc_feature 21: note = DNA
  misc_feature 22..28: note = RNA
  misc_feature 29: note = DNA
  misc_feature 30..38: note = RNA
  misc_feature 39: note = DNA
  misc_feature 40: note = RNA

SEQ ID NO: 140 - DNA, 40 bp, synthetic construct; identical to SEQ ID NO: 107
  source 1..40: mol_type = other DNA
  misc_feature 1..20: note = RNA
  misc_feature 21: note = DNA
  misc_feature 22..28: note = RNA
  misc_feature 29: note = DNA
  misc_feature 30..38: note = RNA
  misc_feature 39..40: note = DNA

SEQ ID NO: 141 - DNA, 40 bp, synthetic construct; identical to SEQ ID NO: 107
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 142 - DNA, 40 bp, synthetic construct; identical to SEQ ID NO: 119
  source 1..40: mol_type = other DNA
  misc_feature 1..20: note = RNA
  misc_feature 21: note = DNA
  misc_feature 22..34: note = RNA
  misc_feature 35: note = DNA
  misc_feature 36..39: note = RNA
  misc_feature 40: note = DNA

SEQ ID NO: 143 - DNA, 40 bp, synthetic construct; identical to SEQ ID NO: 119
  source 1..40: mol_type = other DNA
  misc_feature 1..20: note = RNA
  misc_feature 21: note = DNA
  misc_feature 22..27: note = RNA
  misc_feature 28: note = DNA
  misc_feature 29..34: note = RNA
  misc_feature 35: note = DNA
  misc_feature 36..40: note = RNA

SEQ ID NO: 144 - DNA, 40 bp, synthetic construct; identical to SEQ ID NO: 119
  source 1..40: mol_type = other DNA
  misc_feature 1..27: note = RNA
  misc_feature 28: note = DNA
  misc_feature 29..34: note = RNA
  misc_feature 35: note = DNA
  misc_feature 36..39: note = RNA
  misc_feature 40: note = DNA

SEQ ID NO: 145 - DNA, 40 bp, synthetic construct; identical to SEQ ID NO: 119
  source 1..40: mol_type = other DNA
  misc_feature 1..20: note = RNA
  misc_feature 21: note = DNA
  misc_feature 22..27: note = RNA
  misc_feature 28: note = DNA
  misc_feature 29..38: note = RNA
  misc_feature 39: note = DNA
  misc_feature 40: note = RNA

SEQ ID NO: 146 - DNA, 40 bp, synthetic construct; identical to SEQ ID NO: 119
  source 1..40: mol_type = other DNA
  misc_feature 1..34: note = RNA
  misc_feature 35: note = DNA
  misc_feature 36: note = RNA
  misc_feature 37: note = DNA
  misc_feature 38..39: note = RNA
  misc_feature 40: note = DNA

SEQ ID NO: 147 - DNA, 40 bp, synthetic construct; identical to SEQ ID NO: 119
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 148 - RNA, 40 nt, synthetic construct
  source 1..40: mol_type = other RNA
taatttctactcttgtagatttcggaagagtcagctaatc

SEQ ID NO: 149 - DNA, 40 bp, synthetic construct; identical to SEQ ID NO: 95
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 150 - RNA, 40 nt, synthetic construct
  source 1..40: mol_type = other RNA
taatttctactcttgtagatccatgccaggtcatcacagt

SEQ ID NO: 151 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatccatgccaggtcatcacagt

SEQ ID NO: 152 - RNA, 40 nt, synthetic construct
  source 1..40: mol_type = other RNA
taatttctactcttgtagatgtgtccagttctactctgta

SEQ ID NO: 153 - DNA, 40 bp, synthetic construct; identical to SEQ ID NO: 107
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 154 - RNA, 40 nt, synthetic construct
  source 1..40: mol_type = other RNA
taatttctactcttgtagatggtggctggtggtggaatga

SEQ ID NO: 155 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatggtggctggtggtggaatga

SEQ ID NO: 156 - RNA, 40 nt, synthetic construct
  source 1..40: mol_type = other RNA
taatttctactcttgtagatacttgtagtgtagatgtagt

SEQ ID NO: 157 - DNA, 40 bp, synthetic construct; identical to SEQ ID NO: 119
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA

SEQ ID NO: 158 - RNA, 40 nt, synthetic construct
  source 1..40: mol_type = other RNA
taatttctactcttgtagatgcgaatggcctcctgcagct

SEQ ID NO: 159 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatgcgaatggcctcctgcagct

SEQ ID NO: 160 - RNA, 40 nt, synthetic construct
  source 1..40: mol_type = other RNA
taatttctactcttgtagatgaggagcagctaaccaactt

SEQ ID NO: 161 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatgaggagcagctaaccaactt

SEQ ID NO: 162 - RNA, 40 nt, synthetic construct
  source 1..40: mol_type = other RNA
taatttctactcttgtagatgctctgaaggtgcagaatca

SEQ ID NO: 163 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatgctctgaaggtgcagaatca

SEQ ID NO: 164 - RNA, 40 nt, synthetic construct
  source 1..40: mol_type = other RNA
taatttctactcttgtagattccataagactaagggacaa

SEQ ID NO: 165 - DNA, 40 bp, synthetic construct
  source 1..40: mol_type = other DNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagattccataagactaagggacaa

(19) United States
(12) Patent Application Publication
(10) Pub. No.: US 2025/0325698 A1
(43) Pub. Date: Oct. 23, 2025

(54) THERAPEUTIC APPLICATIONS OF CRISPR TYPE V SYSTEMS

(71) Applicant: Caribou Biosciences, Inc., Berkeley, CA (US)

(72) Inventors: Paul Daniel Donohoue, San Francisco, CA (US); Steven B. Kanner, Los Angeles, CA (US); Antonio Munoz-Howell, Oakland, CA (US); Meghdad Rahdar, Danville, CA (US)

(73) Assignee: Caribou Biosciences, Inc., Berkeley, CA (US)

(21) Appl. No.: 18/855,884

(22) PCT Filed: Apr. 12, 2023
(86) PCT No.: PCT/US2023/065685

§ 371 Date: Oct. 10, 2024

Related U.S. Application Data
(60) Provisional application No. 63/330,695, filed on Apr. 13, 2022.
(60) Provisional application No. 63/332,173, filed on Apr. 18, 2022.

(51) Int. Cl. A61K 48/00; A01K 67/0278; C12N 9/22; C12N 15/11; C12N 15/88; C12N 15/90

(52) U.S. Cl. CPC A61K 48/005; A01K 67/0278; C12N 9/226; C12N 15/111; C12N 15/88; C12N 15/907; A01K 2217/072; A01K 2227/108; A01K 2267/025; C12N 2310/20

(57) ABSTRACT

The present disclosure provides methods and compositions for therapeutic use, where the methods and compositions include Type V CRISPR systems with RNA guides contain ribonucleotide bases and at least one deoxyribonucleotide base. The Type V CRISPR systems are used to perform therapeutic genome editing in somatic cells, induced pluripotency stem cells (iPSCs) and germline or embryonic cells of animals for xenotransplantation of organs and tissues.

### REFERENCE TO RELATED APPLICATIONS

This application claims priority to the U.S. provisional application Ser. No. 63/330,695 filed on Apr. 13, 2022, and the U.S. provisional application Ser. No. 63/332,173 filed on Apr. 18, 2022, all incorporated herein by reference.

### STATEMENT REGARDING FEDERALLY SPONSORED RESEARCH

Not applicable.

### FIELD OF THE INVENTION

The present disclosure relates generally to the field of cellular therapies utilizing cells modified with the Clustered Regularly Interspaced Short Palindromic Repeats (CRISPR) systems, more specifically, CRISPR-Cas12 systems.

### BACKGROUND OF THE INVENTION

The Clustered Regularly Interspaced Short Palindromic Repeats (CRISPR) and CRISPR-associated (Cas) protein systems are found in the genomes of many prokaryotes and provide adaptive immunity against viruses. The state-of-the-art description and classification of various CRISPR-Cas systems in their native hosts (Class 1 Type I; Class 2 Types II and V), RNA targeting (Class 2 Type VI), and joint DNA and RNA targeting (Class 1 Type III) can be found in Makarova et al. (Nat. Rev. Microbiol., 2020, 18:67-83). Of special interest are the Type V systems including different subtypes, e.g., V-A, V-B, V-C, V-D, V-E, V-F, V-G, V-H, V-I, V-J, V-K and V-U. The V-A subtype encodes the Cas12a protein (formerly known as Cpf1). Cas12a has a RuvC-like nuclease domain that is homologous to the respective domain of Cas9 but lacks the HNH nuclease domain.

Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA_33_10 (PeCpf1), Acidaminococcus sp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella sp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1).

CRISPR-Cas systems provide powerful tools for site-directed genome editing by deleting, inserting, mutating, or substituting specific nucleic acid sequences. The alteration can be gene- or location-specific. Genome editing can use site-directed nucleases, such as Cas proteins and their cognate polynucleotides, to cut a target nucleic acid, thereby generating a site for alteration. In certain cases, the cleavage can introduce a double-strand break (DSB) in a target DNA sequence. DSBs can be repaired, e.g., by non-homologous end joining (NHEJ), microhomology-mediated end joining (MMEJ), or homology-directed repair (HDR). HDR relies on the presence of a template for repair. In some examples of this genome editing, a donor polynucleotide or portion thereof can be inserted into the break.

### SUMMARY OF THE INVENTION

This genome editing process utilizing the Type V CRISPR-Cas protein, such as Cas12a in combination with CRISPR hybrid RNA-DNA guides (chRDNAs) is particularly useful for generating genetically-modified cells useful in therapeutic applications.

In some embodiments, the invention is a method of treating a disease or condition characterized by aberrant expression of a gene, the method comprising introducing into a somatic cell of a patient suffering from a disease or condition: (a) a first nucleoprotein complex comprising a Cas 12a protein and a first CRISPR guide molecule having a targeting region capable of binding a first target nucleic acid sequence; and an activating region capable of forming a nucleoprotein complex with the Cas 12a protein, and the Cas 12a protein is capable of cleaving the first target nucleic acid wherein said first CRISPR guide molecule comprises at least one deoxyribonucleotide; and (b) a donor polynucleotide comprising a coding sequence of the gene target aberrantly expressed in individuals suffering from the disease or condition; wherein cleavage by the Cas12a protein results in insertion of the coding sequence into the genome of the somatic cell, and wherein the introducing is by contacting the somatic cells with a lipid nanoparticle comprising the first nucleoprotein complex and the donor polynucleotide, and wherein the gene target is selected from Table 3. In some embodiments, in the CRISPR guide molecule, the activating region, the targeting region, or both comprise at least one deoxyribonucleotide. In some embodiments, the method further comprises introducing into the somatic cell a second nucleoprotein complex comprising a Cas12a protein and a second CRISPR guide molecule having a targeting region capable of binding a second target nucleic acid sequence; and an activating region capable of forming a nucleoprotein complex with the Cas 12a protein, and the Cas 12a protein is capable of cleaving the second target nucleic acid; wherein the coding sequence is inserted between the cleavage sites in first target nucleic acid and the second target nucleic acid cleaved by the Cas 12a protein. In some embodiments, the second CRISPR guide molecule comprises at least one deoxyribonucleotide.

In some embodiments, the insertion of the coding sequence into the genome of the somatic cell results in an increased expression of the gene in the somatic cell. In some embodiments, the lipid nanoparticle comprises one or more cationic lipids with pKₐ of the lipid or combination of two or more lipids is between 6.1 and 6.7. In some embodiments, the lipid nanoparticle comprises a neutral lipid. In some embodiments, the lipid nanoparticle comprises a sterol. In some embodiments, the lipid nanoparticle comprises one or more lipids selected from the group consisting of DSPC, DPPC, POPC, DOPE, SM, PEG-DMA, PEG-DMG, DOTMA, DOSPA, DOTAP, DMRIE, DC-cholesterol, DOTAP-cholesterol, GAP-DMORIE-DPyPE, GL67A-DOPE-DMPE-PEG, 98N12-5, C12-200, DLin-KC2-DMA (KC2), DLin-MC3-DMA (MC3), XTC, MD1, 7C1, PEG-CerC14, and PEG-CerC20.

In some embodiments, the introducing into somatic cells is ex vivo. In some embodiments, the introducing into somatic cells is by systemic intravenous administration, administration into a portal vein, or by intraocular administration.

In some embodiments, the invention is a therapeutic composition for treating a disease or condition characterized by aberrant expression of a gene, the composition comprising: (a) a first nucleoprotein complex comprising a Cas 12a protein and a first CRISPR guide molecule having a targeting region capable of binding a first target nucleic acid sequence; and an activating region capable of forming a nucleoprotein complex with the Cas12a protein, and the Cas12a protein is capable of cleaving the first target nucleic acid wherein said first CRISPR guide molecule comprises at least one deoxyribonucleotide; and (b) a donor polynucleotide comprising a coding sequence of the gene target aberrantly expressed in individuals suffering from the disease or condition; wherein the first nucleoprotein complex and the donor polynucleotide are present in a lipid nanoparticle, and wherein the gene target is selected from Table 3. In some embodiments, in the CRISPR guide molecule, the activating region, the targeting region, or both comprise at least one deoxyribonucleotide.

In some embodiments, the composition further comprises a second nucleoprotein complex comprising a Cas12a protein and a second CRISPR guide molecule having a targeting region capable of binding a second target nucleic acid sequence; and an activating region capable of forming a nucleoprotein complex with the Cas 12a protein, and the Cas 12a protein is capable of cleaving the second target nucleic acid. In some embodiments of the composition, the second CRISPR guide molecule comprises at least one deoxyribonucleotide. In some embodiments of the composition, the lipid nanoparticle comprises one or more cationic lipids with pKₐ of the lipid or combination of two or more lipids is between 6.1 and 6.7. In some embodiments of the composition, the lipid nanoparticle comprises a neutral lipid. In some embodiments of the composition, the lipid nanoparticle comprises a sterol. In some embodiments of the composition, the lipid nanoparticle comprises one or more lipids selected from the group consisting of DSPC, DPPC, POPC, DOPE, SM, PEG-DMA, PEG-DMG, DOTMA, DOSPA, DOTAP, DMRIE, DC-cholesterol, DOTAP-cholesterol, GAP-DMORIE-DPyPE, GL67A-DOPE-DMPE-PEG, 98N12-5, C12-200, DLin-KC2-DMA (KC2), DLin-MC3-DMA (MC3), XTC, MD1, 7C1, PEG-CerC14, and PEG-CerC20. In some embodiments, the composition, further comprises a pharmaceutically acceptable carrier.

In some embodiments, the invention is a method of treating a disease or condition characterized by aberrant expression of a gene with genetically modified differentiated induced pluripotent stem cells (iPSCs), the method comprising (1) introducing into an iPSC: a first nucleoprotein complex comprising a Cas12a protein and a first CRISPR guide molecule having a targeting region capable of binding a first target nucleic acid sequence; and an activating region capable of forming a nucleoprotein complex with the Cas12a protein, and the Cas12a protein is capable of cleaving the first target nucleic acid wherein said first CRISPR guide molecule comprises at least one deoxyribonucleotide; wherein cleavage by the Cas 12a protein results in a modification of a gene target selected from Table 4 or Table 5; (2) differentiating the iPSC into a cell type affected by the disease or condition in individuals suffering from the disease or condition; and (3) administering the differentiated iPSC to a patient affected by the disease or condition. In some embodiments, in the CRISPR guide molecule, the activating region, the targeting region, or both comprise at least one deoxyribonucleotide.

In some embodiments, the method further comprises introducing into the iPSC a second nucleoprotein complex comprising a Cas12a protein and a second CRISPR guide molecule having a targeting region capable of binding a second target nucleic acid sequence; and an activating region capable of forming a nucleoprotein complex with the Cas12a protein, and the Cas12a protein is capable of cleaving the second target nucleic acid; wherein the coding sequence is inserted between the cleavage sites in first target nucleic acid and the second target nucleic acid cleaved by the Cas 12a protein. In some embodiments of the method, the second CRISPR guide molecule comprises at least one deoxyribonucleotide. In some embodiments, the method further comprises introducing into the iPSC a donor polynucleotide comprising a coding sequence of the gene target selected from Table 4. In some embodiments of the method, the cleavage with the Cas12a protein results in an insertion of the coding sequence into the genome of the iPSC. In some embodiments of the method, the insertion of the coding sequence into the genome of the iPSC results in an increased expression of the gene in the iPSC. In some embodiments of the method, the cleavage with the Cas 12a protein results in a disruption in the genome of the iPSC of a coding sequence of a gene target listed in Table 5. In some embodiments of the method, the disruption in the genome of the iPSC results in a decreased expression of the gene in the iPSC. In some embodiments of the method, the iPSC is produced by reprogramming a somatic cell. In some embodiments of the method, the reprogramming is by inducing expression of one or more genes in the somatic cell. In some embodiments of the method, the reprogramming is by inducing gene expression is by introducing an mRNA into the somatic cell. In some embodiments of the reprogramming, the one or more genes is selected from of Oct4, Sox2, Klf4, c-Myc, NANOG, Sox1, Sox3, Sox 15, Sox18, Klf1, Klf2, Klf5, NR5A2, c-Myc, 1-Myc, n-Myc, Rem2, Tert, LIN28, and Wnt. In some embodiments of the reprogramming, the one or more genes consists of a combination of Oct4, Sox2, Klf4, and c-Myc. In some embodiments of the reprogramming, the one or more genes consists of a combination of Oct4, Sox2, and NANOG. In some embodiments of the method, the reprogramming further comprises contacting the iPSCs with one or more of MEK inhibitor, a DNA methyltransferase inhibitor, a histone deacetylase (HDAC) inhibitor, valproic acid, 5′-azacytidine, dexamethasone, suberoylanilide, hydroxamic acid (SAHA), vitamin C, and trichostatin (TSA) Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylurcido) caproic hydroxamic acid), AOE (2-amino-8-oxo-9, 10-epoxydecanoic acid), CHAP31 and CHAP50. In some embodiments of the method, the iPSC are differentiated into neurons. In some embodiments of the method, the iPSC are differentiated into neurons by incubating the iPSCs in the presence of one or more of GSK-3 inhibitors, TGF-beta receptor, or TGF-beta inhibitors, ALK inhibitors, dorsomorphin, compound E, FGF, EGF, all-trans-retinoic acid, Sonic Hedgehog protein, purmorphamine, SAG dihydrochloride, CNTF, and GDNF. In some embodiments of the method, the differentiation of iPSC into neurons is assessed by measuring expression of one or more of Sox1, Pax6, Nestin, HB9, MAP2, NeuroFilament, Tuj1 and Olig2 after the differentiation process. In some embodiments of the method, the differentiation of iPSC into neurons is assessed by measuring electrical activity of the cells after the differentiation process.

In some embodiments of the method, the iPSC are differentiated into myocytes. In some embodiments of the method, the iPSC are differentiated into myocytes by incubating the iPSCs in the presence of one or more of GSK-3 inhibitor, and a Wnt-dependent phosphorylation blocker. In some embodiments of the method, the differentiation of iPSC into myocytes is assessed by measuring expression of one or more of TBX5, TNNT2, MYH6 and MYL7 after the differentiation process.

In some embodiments, the invention is a composition treating a disease or condition characterized by aberrant expression of a gene with genetically modified differentiated induced pluripotent stem cells (iPSCs), comprising an iPSC comprising: a first nucleoprotein complex comprising a Cas12a protein and a first CRISPR guide molecule having a targeting region capable of binding a first target nucleic acid sequence; and an activating region capable of forming a nucleoprotein complex with the Cas 12a protein, and the Cas 12a protein is capable of cleaving the first target nucleic acid wherein said first CRISPR guide molecule comprises at least one deoxyribonucleotide; wherein cleavage by the Cas12a protein results in a modification of a gene target selected from Table 4 or Table 5; and the iPSC is capable of differentiating into a cell type affected by the disease or condition in individuals suffering from the disease or condition. In some embodiments of the composition, CRISPR guide molecule, the activating region, the targeting region, or both comprise at least one deoxyribonucleotide. In some embodiments the composition further comprises introducing into the iPSC a second nucleoprotein complex comprising a Cas 12a protein and a second CRISPR guide molecule having a targeting region capable of binding a second target nucleic acid sequence; and an activating region capable of forming a nucleoprotein complex with the Cas 12a protein, and the Cas 12a protein is capable of cleaving the second target nucleic acid; wherein the coding sequence is inserted between the cleavage sites in first target nucleic acid and the second target nucleic acid cleaved by the Cas12a protein. In some embodiments of the composition, the second CRISPR guide molecule comprises at least one deoxyribonucleotide. In some embodiments, the composition further comprises a donor polynucleotide comprising a coding sequence of the gene target selected from Table 4. In some embodiments of the composition, the cleavage with the Cas12a protein results in an insertion of the coding sequence into the genome of the iPSC. In some embodiments of the composition, the insertion of the coding sequence into the genome of the iPSC results in an increased expression of the gene in the iPSC. In some embodiments of the composition, the cleavage with the Cas 12a protein results in a disruption in the genome of the iPSC of a coding sequence of a gene target listed in Table 5. In some embodiments of the composition, the disruption in the genome of the iPSC results in a decreased expression of the gene in the iPSC. In some embodiments of the composition, the iPSC is produced by reprogramming a somatic cell. In some embodiments of the composition, the reprogramming is by inducing expression of one or more genes in the somatic cell. In some embodiments of the composition, the reprogramming is by inducing gene expression is by introducing an mRNA into the somatic cell. In some embodiments of the composition, the one or more genes is selected from of Oct4, Sox2, Klf4, c-Myc, NANOG, Sox1, Sox3, Sox15, Sox 18, Klf1, Klf2, Klf5, NR5A2, c-Myc, 1-Myc, n-Myc, Rem2, Tert, LIN28, and Wnt. In some embodiments of the composition, the one or more genes consists of a combination of Oct4, Sox2, Klf4, and c-Myc. In some embodiments of the composition, the one or more genes consists of a combination of Oct4, Sox2, and NANOG. In some embodiments of the composition, the reprogramming further comprises contacting the iPSCs with one or more of MEK inhibitor, a DNA methyltransferase inhibitor, a histone deacetylase (HDAC) inhibitor, valproic acid, 5′-azacytidine, dexamethasone, suberoylanilide, hydroxamic acid (SAHA), vitamin C, and trichostatin (TSA) Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylurcido) caproic hydroxamic acid), AOE (2-amino-8-oxo-9, 10-epoxydecanoic acid), CHAP31 and CHAP50.

In some embodiments of the composition, the iPSC are differentiated into neurons. In some embodiments of the composition, the iPSC are differentiated into neurons by incubating the iPSCs in the presence of one or more of GSK-3 inhibitors, TGF-beta receptor, or TGF-beta inhibitors, ALK inhibitors, dorsomorphin, compound E, FGF, EGF, all-trans-retinoic acid, Sonic Hedgehog protein, purmorphamine, SAG dihydrochloride, CNTF, and GDNF. In some embodiments of the composition, the differentiation of iPSC into neurons is assessed by measuring expression of one or more of Sox1, Pax6, Nestin, HB9, MAP2, NeuroFilament, Tuj1, and Olig2 after the differentiation process. In some embodiments of the composition, the differentiation of iPSC into neurons is assessed by measuring electrical activity of the cells after the differentiation process.

In some embodiments of the composition, the iPSC are differentiated into myocytes. In some embodiments of the composition, the iPSC are differentiated into myocytes by incubating the iPSCs in the presence of one or more of GSK-3 inhibitor, and a Wnt-dependent phosphorylation blocker. In some embodiments of the composition, the differentiation of iPSC into myocytes is assessed by measuring expression of one or more of TBX5, TNNT2, MYH6 and MYL7 after the differentiation process.

In some embodiments, the composition further comprises a pharmaceutically acceptable carrier.

In some embodiments, the invention is a method of making genetically modified differentiated induced pluripotent stem cells (iPSCs) for treating a disease or condition characterized by aberrant expression of a gene, the method comprising (1) introducing into an iPSC: a first nucleoprotein complex comprising a Cas12a protein and a first CRISPR guide molecule having a targeting region capable of binding a first target nucleic acid sequence; and an activating region capable of forming a nucleoprotein complex with the Cas 12a protein, and the Cas 12a protein is capable of cleaving the first target nucleic acid wherein said first CRISPR guide molecule comprises at least one deoxyribonucleotide; wherein cleavage by the Cas12a protein results in a modification of a gene target selected from Table 4 or Table 5; (2) differentiating the iPSC into a cell type affected by the disease or condition in individuals suffering from the disease or condition; and (3) administering the differentiated iPSC to a patient affected by the disease or condition. In some embodiments of the method, the CRISPR guide molecule, the activating region, the targeting region, or both comprise at least one deoxyribonucleotide. In some embodiments, the method further comprises introducing into the iPSC a second nucleoprotein complex comprising a Cas12a protein and a second CRISPR guide molecule having a targeting region capable of binding a second target nucleic acid sequence; and an activating region capable of forming a nucleoprotein complex with the Cas 12a protein, and the Cas 12a protein is capable of cleaving the second target nucleic acid; wherein the coding sequence is inserted between the cleavage sites in first target nucleic acid and the second target nucleic acid cleaved by the Cas 12a protein. In some embodiments of the method, the second CRISPR guide molecule comprises at least one deoxyribonucleotide. In some embodiments, method further comprises introducing into the iPSC a donor polynucleotide comprising a coding sequence of the gene target selected from Table 4. In some embodiments of the method, the cleavage with the Cas 12a protein results in an insertion of the coding sequence into the genome of the iPSC. In some embodiments of the method, the insertion of the coding sequence into the genome of the iPSC results in an increased expression of the gene in the iPSC. In some embodiments of the method, the cleavage with the Cas12a protein results in a disruption in the genome of the iPSC of a coding sequence of a gene target listed in Table 5. In some embodiments of the method, the disruption in the genome of the iPSC results in decreased expression of the gene in the iPSC. In some embodiments of the method, the iPSC is produced by reprogramming a somatic cell. In some embodiments of the method, the reprogramming is by inducing expression of one or more genes in the somatic cell. In some embodiments of the method, the reprogramming is by inducing gene expression is by introducing an mRNA into the somatic cell. In some embodiments of the reprogramming, the one or more genes is selected from of Oct4, Sox2, Klf4, c-Myc, NANOG, Sox1, Sox3, Sox 15, Sox 18, Klf1, Klf2, KIf5, NR5A2, c-Myc, 1-Myc, n-Myc, Rem2, Tert, LIN28, and Wnt. In some embodiments of the reprogramming, the one or more genes consists of a combination of Oct4, Sox2, Klf4, and c-Myc. In some embodiments of the reprogramming, the one or more genes consists of a combination of Oct4, Sox2, and NANOG. In some embodiments of the method, the reprogramming further comprises contacting the iPSCs with one or more of MEK inhibitor, a DNA methyltransferase inhibitor, a histone deacetylase (HDAC) inhibitor, valproic acid, 5′-azacytidine, dexamethasone, suberoylanilide, hydroxamic acid (SAHA), vitamin C, and trichostatin (TSA) Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylurcido) caproic hydroxamic acid), AOE (2-amino-8-oxo-9, 10-epoxydecanoic acid), CHAP31 and CHAP50.

In some embodiments of the method, the iPSC are differentiated into neurons. In some embodiments of the method, the iPSC are differentiated into neurons by incubating the iPSCs in the presence of one or more of GSK-3 inhibitors, TGF-beta receptor, or TGF-beta inhibitors, ALK inhibitors, dorsomorphin, compound E, FGF, EGF, all-trans-retinoic acid, Sonic Hedgehog protein, purmorphamine, SAG dihydrochloride, CNTF, and GDNF. In some embodiments of the method, the differentiation of iPSC into neurons is assessed by measuring expression of one or more of Sox1, Pax6, Nestin, HB9, MAP2, NeuroFilament, Tuj1, and Olig2 after the differentiation process. In some embodiments of the method, the differentiation of iPSC into neurons is assessed by measuring electrical activity of the cells after the differentiation process.

In some embodiments of the method, the iPSC are differentiated into myocytes. In some embodiments of the method, the iPSC are differentiated into myocytes by incubating the iPSCs in the presence of one or more of GSK-3 inhibitor, and a Wnt-dependent phosphorylation blocker. In some embodiments of the method, the differentiation of iPSC into myocytes is assessed by measuring expression of one or more of TBX5, TNNT2, MYH6 and MYL7 after the differentiation process.

In some embodiments, the invention is a method of making a transgenic animal for xenotransplantation, the method comprising: (1) introducing into a cell of an animal: a first nucleoprotein complex comprising a Cas12a protein and a first CRISPR guide molecule having a targeting region capable of binding a first target nucleic acid sequence; and an activating region capable of forming a nucleoprotein complex with the Cas 12a protein, and the Cas 12a protein is capable of cleaving the first target nucleic acid wherein said first CRISPR guide molecule comprises at least one deoxyribonucleotide; wherein cleavage by the Cas12a protein results in a modification of a gene target selected from Table 6; (2) introducing the cell into a foster female animal. In some embodiments of the method, the cell of an animal is an oocyte, ovum, or zygote. In some embodiments of the method, the cell of an animal is a somatic cell and the method further comprises after step (1), transferring the nucleus of the cell into an enucleated ovum or zygote. In some embodiments of the method, the animal is a pig. In some embodiments of the method, in the CRISPR guide molecule, the activating region, the targeting region, or both comprise at least one deoxyribonucleotide. In some embodiments, the method further comprises introducing into the iPSC a second nucleoprotein complex comprising a Cas 12a protein and a second CRISPR guide molecule having a targeting region capable of binding a second target nucleic acid sequence; and an activating region capable of forming a nucleoprotein complex with the Cas12a protein, and the Cas 12a protein is capable of cleaving the second target nucleic acid; wherein the coding sequence is inserted between the cleavage sites in first target nucleic acid and the second target nucleic acid cleaved by the Cas 12a protein. In some embodiments of the method, the second CRISPR guide molecule comprises at least one deoxyribonucleotide. In some embodiments, the method further comprises introducing into the cell a donor polynucleotide comprising a coding sequence of the gene target selected from A20, HO-1, FAT-1, TNF-alpha receptor, CD39, hirudin, TFPI, EPCR, TBM, CD46, DAF (CD55), CD59, CR1, CTLA4, CD47, one or more of Class I HLA. In some embodiments of the method, the cleavage with the Cas12a protein results in an insertion of the coding sequence into the genome of the cell. In some embodiments of the method, the insertion of the coding sequence into the genome of the cell results in an increased expression of the gene in the cell. In some embodiments of the method, the cleavage with the Cas 12a protein results in a disruption in the genome of the cell of a coding sequence of a gene target selected from GGTA1, b4GalNT2, CMAH, GT (alpha (1,3)-galactosyltransferase), GHR, one or more of Class I SLA. In some embodiments of the method, the disruption in the genome of the cell results in a decreased expression of the gene in the cell.

In some embodiments, the invention is a composition for making a transgenic animal for xenotransplantation, comprising an animal cell comprising: a first nucleoprotein complex comprising a Cas 12a protein and a first CRISPR guide molecule having a targeting region capable of binding a first target nucleic acid sequence; and an activating region capable of forming a nucleoprotein complex with the Cas 12a protein, and the Cas 12a protein is capable of cleaving the first target nucleic acid wherein said first CRISPR guide molecule comprises at least one deoxyribonucleotide; wherein cleavage by the Cas12a protein results in a modification of a gene target selected from Table 6. In some embodiments of the composition, the cell of an animal is an oocyte, ovum, or zygote. In some embodiments of the composition, the cell of an animal is an ovum or zygote resulting from a transfer of a nucleus of a somatic cell into an enucleated ovum or zygote. In some embodiments of the composition, the animal is a pig. In some embodiments of the composition, the CRISPR guide molecule, the activating region, the targeting region, or both comprise at least one deoxyribonucleotide. In some embodiments, the composition further comprises introducing into the iPSC a second nucleoprotein complex comprising a Cas12a protein and a second CRISPR guide molecule having a targeting region capable of binding a second target nucleic acid sequence; and an activating region capable of forming a nucleoprotein complex with the Cas 12a protein, and the Cas12a protein is capable of cleaving the second target nucleic acid; wherein the coding sequence is inserted between the cleavage sites in first target nucleic acid and the second target nucleic acid cleaved by the Cas12a protein. In some embodiments of the composition, the second CRISPR guide molecule comprises at least one deoxyribonucleotide. In some embodiments, the composition further comprises a donor polynucleotide comprising a coding sequence of the gene target selected from A20, HO-1, FAT-1, TNF-alpha receptor, CD39, hirudin, TFPI, EPCR, TBM, CD46, DAF (CD55), CD59, CR1, CTLA4, CD47, one or more of Class I HLA. In some embodiments of the composition, the cleavage with the Cas12a protein results in an insertion of the coding sequence into the genome of the cell. In some embodiments of the composition, the insertion of the coding sequence into the genome of the cell results in an increased expression of the gene in the cell. In some embodiments of the composition, the cleavage with the Cas 12a protein results in a disruption in the genome of the cell of a coding sequence of a gene target selected from GGTA1, b4GalNT2, CMAH, GT (alpha (1,3)-galactosyltransferase), GHR, one or more of Class I SLA. In some embodiments of the composition, the disruption in the genome of the cell results in a decreased expression of the gene in the cell.

### BRIEF DESCRIPTION OF THE FIGURES

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying figures. The figures are not proportionally rendered, nor are they to scale. The locations of indicators are approximate.

FIG. 1A, FIG. 1B, and FIG. 1C illustrate examples of Type V CRISPR-Cas12a guide RNAs.

FIG. 2 illustrates a Cas12a chRDNA guide/nucleoprotein complex cleavage of a target polynucleotide.

FIG. 3A-FIG. 3I illustrate various canonical and non-canonical nucleotides for use in Cas12 chRDNA guides.

FIG. 4 illustrates a Cas12a chRDNA guide/nucleoprotein complex cleavage of a target polynucleotide.

FIG. 5 illustrates a Cas12a crRNA guide.

FIG. 6 illustrates a Cas12a chRDNA guide comprising DNA bases in the activating region and target binding sequence.

FIG. 7 illustrates a Cas12a chRDNA guide comprising DNA bases and chemically modified nucleic acids in the activating region and target binding sequence.

FIG. 8 illustrates the formation of a Cas12 chRDNA guide/nucleoprotein complex and binding of a target polynucleotide.

FIG. 9 illustrates the generation of insertion or deletions (indels) in a target polynucleotide by a Cas12 chRDNA guide/nucleoprotein complex.

FIG. 10 illustrates the insertion of a donor polynucleotide sequence in a target polynucleotide by a Cas12 chRDNA guide/nucleoprotein complex.

FIG. 11 illustrates nicking of a target polynucleotide by a Cas12 chRDNA guide/nucleoprotein complex.

FIG. 12 illustrates the tandem nicking of a target polynucleotide with two Cas12 chRDNA guide/nucleoprotein complexes and insertion of a donor polynucleotide sequence in a target polynucleotide.

### DETAILED DESCRIPTION OF THE INVENTION

### Definitions

The following definitions aid in understanding this disclosure.

The terms “guide” and “guide polynucleotide” as used herein refer to one or more polynucleotides that form a nucleoprotein complex with a Cas protein, wherein the nucleoprotein complex preferentially binds a nucleic acid target sequence in a polynucleotide (relative to a polynucleotide that does not comprise the nucleic acid target sequence). Such guides can comprise ribonucleotide bases (e.g., RNA), deoxyribonucleotide bases (e.g., DNA), combinations of ribonucleotide bases and deoxyribonucleotide bases (e.g., RNA/DNA), nucleotide analogs, modified nucleotides, and the like, as well as synthetic, naturally occurring, and non-naturally occurring modified backbone residues or linkages. Many such guides are known, such as but not limited to, single-guide RNA (including miniature and truncated single-guide RNAs), crRNA, dual-guide RNAs, including but not limited to, crRNA/tracrRNA molecules, and the like, the use of which depends on the particular Cas protein. For example, a “Type V CRISPR-Cas 12-associated guide” is a guide that specifically associates with a cognate Cas12 protein to form a nucleoprotein complex.

As used herein, a “CRISPR polynucleotide” is a polynucleotide sequence comprising a portion of a guide molecule. In some embodiments, the CRISPR polynucleotide includes a targeting region and/or an activating region.

With reference to a guide molecule, a “spacer,” “spacer sequence,” “spacer element,” or “targeting region,” as used herein refers to a polynucleotide sequence that can specifically hybridize to a target nucleic acid sequence. The targeting region interacts with the target nucleic acid sequence through hydrogen bonding between complementary base pairs (i.e., paired bases). A targeting region binds to a selected nucleic acid target sequence. In some embodiments, the target sequence is a sequence within a genome of a cell, either in vitro, ex vivo (such as in the generation of CAR-T cells), or in vivo (such as where compositions are administered directly to a subject). The targeting region determines the location of the site-specific binding and nucleolytic cleavage of a Cas 12 protein. Variability of the functional length for a targeting region is known in the art.

With reference to a guide molecule, the term “activating region” refers to a portion of a polynucleotide capable of associating, or binding with, a Cas12 polypeptide, such as a Cas12a polypeptide.

As used herein, the terms “base analog,” “non-canonical base,” and “chemically-modified base” refer to a compound having structural similarity to a canonical purine or pyrimidine base occurring in DNA or RNA. The base analog may contain a modified sugar and/or a modified nucleobase, as compared to a purine or pyrimidine base occurring naturally in DNA or RNA. In some embodiments, the base analog is inosine or deoxyinosine, such as 2′-deoxyinosine. In other embodiments, the base analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuridine, iso deoxycytidine, other 0-1 purine analogs, N-6-hydroxylaminopurine, nebularine, 7-deaza hypoxanthine, other 7-deazapurines, and 2-methyl purines). In some embodiments, the base analog may be selected from the group consisting of 7-deaza-2′-deoxyinosine, 2′-aza-2′-deoxyinosine, PNA-inosine, morpholino-inosine, LNA-inosine, phosphoramidite-inosine, 2′-O-methoxyethyl-inosine, and 2′-OMe-inosine. The term “base analog” also includes, for example, 2′-deoxyribonucleosides, 2′-ribonucleosides, 2′-deoxyribonucleotides or 2′-ribonucleotides, wherein the nucleobase is a substituted hypoxanthine. For instance, the substituted hypoxanthine may be substituted with a halogen, such as fluorine or chlorine. In some embodiments, the base analog may be a fluoroinosine or a chloroinosine, such as 2-chloroinosine, 6-chloroinosine, 8-chloroinosine, 2-fluoroinosine, 6-fluoroinosine, or 8-fluoroinosine. In other embodiments, the base analog is deoxyuridine. In other embodiments the base analog is a nucleic acid mimic (such as, for example, artificial nucleic acids and xeno nucleic acids (XNA)).

As used herein, the term “CRISPR hybrid RNA/DNA guide” (chRDNA) refers to a polynucleotide guide molecule comprising a targeting region, wherein the polynucleotide comprises RNA with DNA designed into the polynucleotide.

As used herein, the term “Cas12-chRDNA guide nucleoprotein complex” refers to a chRDNA guide molecule complexed with a Cas12 protein to form a nucleoprotein complex, wherein the nucleoprotein complex is capable of site-directed binding to a nucleic acid target sequence complementary to the nucleic acid target binding sequence present in the chRDNA guide molecule.

A “linker element nucleotide sequence,” “linker nucleotide sequence,” and “linker polynucleotide” are used interchangeably herein and refer to a sequence of one or more nucleotides covalently attached to a first nucleic acid sequence (5′-linker nucleotide sequence-first nucleic acid sequence-3′). In some embodiments, a linker nucleotide sequence connects two separate nucleic acid sequences to form a single polynucleotide (e.g., 5′-first nucleic acid sequence-linker nucleotide sequence-second nucleic acid sequence-3′).

As used herein, the term “cognate” typically refers to a Cas12 protein (e.g., Cas12a) and one or more Type V CRISPR-Cas12-associated guides (e.g., Cas12 chRDNA guides) that are capable of forming a nucleoprotein complex capable of site-directed binding to a nucleic acid target sequence complementary to the nucleic acid target binding sequence present in one of the one or more guides.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

As used herein, a Cas 12 protein is said to “target” a polynucleotide if a Cas12 guide/nucleoprotein complex binds or cleaves a polynucleotide at the nucleic acid target sequence within the polynucleotide.

A “protospacer adjacent motif” or “PAM” as used herein refers to double-stranded nucleic acid sequences comprising a Cas12 protein-binding recognition sequence, wherein amino acids of the Cas 12 protein directly interact with the recognition sequence (e.g., Cas12a protein interacts with the PAM 5′-TTTN-3′ or the PAM 5′-TTTV-3′). PAM sequences are on the non-target strand and can be 5′ or 3′ of a target complement sequence (e.g., in CRISPR-Cas12a systems the PAM 5′-TTTN-3′ or the PAM 5′-TTTV-3′sequence is on the non-target strand and is 5′ of the target-complement sequence).

“Target,” “target sequence,” “nucleic acid target sequence,” “target nucleic acid sequence,” and “on-target sequence” are used interchangeably herein to refer to a nucleic acid sequence that is wholly, or in part, complementary to a nucleic acid target binding sequence of a Cas12 polynucleotide (e.g., the targeting region). Typically, the nucleic acid target binding sequence is selected to be 100% complementary to a nucleic acid target sequence to which binding of a Cas12 nucleoprotein complex is being directed; however, to attenuate binding to a nucleic acid target sequence, lower percent complementarity can be used.

“Donor polynucleotide,” “donor oligonucleotide,” “donor template,” “non-viral donor,” and “non-viral template” are used interchangeably herein and can be a double-stranded polynucleotide (e.g., DNA), a single-stranded polynucleotide (e.g., DNA or RNA), or a combination thereof. Donor polynucleotides can comprise homology arms flanking the insertion sequence (e.g., DSBs in the DNA). The homology arms on each side can vary in length to ensure the desirable level of hybridization at the conditions used.

As used herein, “homology-directed repair” (HDR) refers to DNA repair that takes place in cells, for example, during repair of a DSB in DNA. HDR requires nucleotide sequence homology and uses a donor polynucleotide to repair the sequence wherein the DSB (e.g., within a target DNA sequence) occurred. For example, a donor polynucleotide can be used for repair of the break in the target DNA sequence, wherein the repair results in the transfer of genetic information (e.g., polynucleotide sequences) from the donor polynucleotide at the site or in close proximity of the break in the DNA. Accordingly, new genetic information (e.g., polynucleotide sequences) may be inserted or copied at a target DNA sequence.

As used herein, “homology-independent target integration” (HITI) refers to DNA repair that takes place in a cell, for example, during repair of a DSB in DNA. HITI, unlike HDR, does not require nucleotide sequence homology and uses a donor polynucleotide to repair the sequence wherein the DSB occurred (e.g., within a target DNA sequence). HITI results in the transfer of genetic information from, for example, the donor polynucleotide to the target DNA sequence. Accordingly, new genetic information (e.g., polynucleotide sequences) may be inserted or copied at a target DNA sequence.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions.

As used herein, “non-homologous end joining” (NHEJ) refers to the repair of a DSB in DNA by direct ligation of one terminus of the break to the other terminus of the break without a requirement for a donor polynucleotide. NHEJ is a DNA repair pathway available to cells to repair DNA without the use of a repair template. NHEJ in the absence of a donor polynucleotide often results in nucleotides being randomly inserted or deleted at the site of the DSB.

“Microhomology-mediated end joining” (MMEJ) is pathway for repairing a DSB in DNA. MMEJ involves deletions flanking a DSB and alignment of microhomologous sequences internal to the break site before joining. MMEJ is genetically defined and requires the activity of, for example, CtIP, Poly (ADP-Ribose) Polymerase 1 (PARP1), DNA polymerase theta (Pol θ), DNA Ligase 1 (Lig 1), or DNA Ligase 3 (Lig 3). Additional genetic components are known in the art. See, e.g., Sfeir et al. (Trends in Biochemical Sciences, 2015, 40:701-714).

As used herein, “DNA repair” encompasses any process whereby cellular machinery repairs damage to a DNA molecule contained in the cell. The damage repaired can include single-strand breaks or double-strand breaks (DSBs). At least three mechanisms exist to repair DSBs: HDR, NHEJ, and MMEJ. “DNA repair” is also used herein to refer to DNA repair resulting from human manipulation, wherein a target locus is modified, e.g., by inserting, deleting, or substituting nucleotides, all of which represent forms of genome editing.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

As used herein, the term “operably linked” refers to polynucleotide sequences or amino acid sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function if separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, the term “modulate” refers to a change in the quantity, degree or amount of a function. For example, a Cas 12-guide/nucleoprotein complex, as disclosed herein, may modulate the activity of a promoter sequence by binding to a nucleic acid target sequence at or near the promoter. Depending on the action occurring after binding, the Cas 12 guide/nucleoprotein complex can induce, enhance, suppress, or inhibit, transcription of a gene operatively linked to the promoter sequence. Thus, “modulation” of gene expression includes both gene activation and gene repression.

An “adoptive cell” refers to a cell that can be or has been genetically modified for use in a cell therapy treatment.

A “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells are embryonic, fetal, amniotic, adult, or induced pluripotent stem cells.

An “induced pluripotent stem cell” (iPSCs) refers to a type of pluripotent stem cell that is artificially derived from a non-pluripotent cell, typically a somatic cell. Pluripotent stem cells can be edited before or after differentiation, with a Cas12 chRDNA guide/nucleoprotein complex. An iPSC can be further modified, before or after differentiation, through the introduction of an exogenous gene or sequence into the genome, such as sequence encoding a CAR.

A “hematopoietic stem cell” refers to an undifferentiated cell that has the ability to differentiate into a cell of hematopoietic lineage, such as a lymphocyte.

A “lymphocyte” refers to a leukocyte (white blood cell) that is part of the vertebrate immune system. Also encompassed by the term “lymphocyte” is a hematopoietic stem cell that gives rise to lymphoid cells. Lymphocytes include T cells for cell-mediated, cytotoxic adaptive immunity, such as CD4+ and/or CD8+ cytotoxic T cells; alpha/beta T cells and gamma/delta T cells; regulatory T cells such as Treg cells; natural killer (NK) cells that function in cell-mediated, cytotoxic innate immunity; and B cells, for humoral, antibody-driven adaptive immunity; NK/T cells; cytokine induced killer cells (CIK cells); and antigen presenting cells (APCs), such as dendritic cells. The lymphocyte can be a mammalian cell, such as a human cell.

Tumor infiltrating lymphocytes (TILs) are also encompassed by the term “lymphocyte” as used herein. TILs are immune cells that have penetrated the environment in and around a tumor (the “tumor microenvironment”). The term “lymphocyte” also encompasses genetically-modified T cells and NK cells (CAR-T cells and CAR-NK cells).

The terms “subject,” “individual,” or “patient” are used interchangeably herein and refer to humans and other primates, and other mammals, farm animals, domestic mammals, and laboratory animals. In some embodiments, a cell is derived from a subject (for example, lymphocytes, stem cells, progenitor cells, or tissue-specific cells). In some embodiments, the subject is a non-human subject.

The terms “effective amount” or “therapeutically effective amount” of a composition or agent, such as a genetically engineered adoptive cell as provided herein, refer to a sufficient amount of the composition or agent to provide the desired response. Preferably, the effective amount will prevent, avoid, or eliminate one or more harmful side-effects. Such responses will depend on the particular disease in question. For example, in a patient being treated for cancer using an adoptive cell therapy, a desired response may include, prevent, avoid, or eliminate, one or more of: treatment or prevention of the effects of graft versus host disease (GvHD), host versus graft rejection, cytokine release syndrome (CRS), cytokine storm, and the reduction of oncogenic transformations of administered genetically-modified cells. The exact treatment amount required will vary from subject to subject, depending on the species, age, and general condition of the subject, the severity of the condition being treated, and the particular modified lymphocyte used, mode of administration, and the like. An appropriate “effective” amount in any individual case may be determined by one of ordinary skill in the art using routine experimentation.

“Treatment” or “treating” a particular disease, such as a cancerous condition or GvHD, includes: preventing the disease, for example, preventing the development of the disease or causing the disease to occur with less intensity in a subject that may be predisposed to the disease, but does not yet experience or display symptoms of the disease; inhibiting the disease, for example, reducing the rate of development, arresting the development, or reversing the disease state; and/or relieving symptoms of the disease, for example, decreasing the number of symptoms experienced by the subject.

The present disclosure relies on the ordinary skill in the art as it pertains conventional techniques of immunology, biochemistry, chemistry, molecular biology, microbiology, cell biology, genomics, and recombinant polynucleotides, as taught, for example, by the following standard publication: Sambrook, Joseph. Molecular Cloning: a Laboratory Manual. Cold Spring Harbor, N.Y., Cold Spring Harbor Laboratory Press, 2001; E. A. Greenfield (Antibodies: A Laboratory Manual, 2014, Second edition, Cold Spring Harbor Laboratory Press, ISBN 978-1-936113-81-1); R.I. Freshney (Culture of Animal Cells: A Manual of Basic Technique and Specialized Applications, 2016, 7th Edition, Wiley-Blackwell, ISBN 978-1118873656); J. M. Walker (Methods in Molecular Biology (Series), Humana Press, ISSN 1064-3745); Green et al. (Molecular Cloning: A Laboratory Manual, 2012, Fourth Edition, Cold Spring Harbor Laboratory Press, ISBN 978-1605500560).

### CRISPR Type V Systems

Clustered regularly interspaced short palindromic repeats (CRISPR) and related CRISPR-associated proteins (Cas proteins) constitute CRISPR-Cas systems. The classification of CRISPR-Cas systems has had many iterations. Makarova et al. (Nat. Rev. Microbiol., 2020, 18:67-83) proposed a classification system that takes into consideration the signature cas genes specific for individual types and subtypes of CRISPR-Cas systems. The classification also considered sequence similarity between multiple shared Cas proteins, the phylogeny of the best-conserved Cas protein, gene organization, and the structure of the CRISPR array. This approach provided a classification scheme that divides CRISPR-Cas systems into two distinct classes: Class 1 and Class 2.

In Class 2, Type V, systems, the crRNA and target binding involves Cas12, as does the target nucleic acid cleavage. The RuvC-like nuclease domain of Cas12a, for instance, cleaves both strands of the target nucleic acid in a staggered configuration, producing 5′ overhangs, which is in contrast to the blunt ends generated by Cas9 cleavage. These 5′ overhangs may facilitate insertion of DNA through homologous recombination methods.

Other proteins associated with Type V crRNA and target binding and cleavage include Cas12b (formerly C2c1) and Cas12c (formerly C2c3). Cas12b and Cas12c proteins are similar in length to CRISPR Class 2 Type II Cas9 and CRISPR Class 2 Type V Cas12a proteins, ranging from approximately 1,100 amino acids to approximately 1,500 amino acids. C2c1 and C2c3 proteins also contain RuvC-like nuclease domains and have an architecture similar to Cas12a. C2c1 proteins are similar to Cas9 proteins in requiring a crRNA and a tracrRNA for target binding and cleavage, but have an optimal cleavage temperature of 50° C. C2c1 proteins target an AT-rich PAM, which similar to Cas12a, is 5′ of the target sequence. See, e.g., Shmakov et al. (Molecular Cell, 2015, 60 (3): 385-397).

The CRISPR Type V subtypes include the Cas12 proteins and demonstrate a broad sequence and diversity in size; however, Cas12 subtypes share a common evolutionary origin from TnpB nucleases encoded by IS605-like transposons. Owing to the low sequence similarity, and likely evolution through multiple independent recombination events of Cas12 proteins, classification of Cas12 proteins into their respective subtypes has resulted in multiple naming conventions. Table 1 presents the classification and names for the Type V Cas12 proteins as well as their approximate size, guide requirements, preferred target polynucleotide, and a representative organism of origin.

TABLE 1
Classification of Type V Subtypes

| Type | Cas | Other | Effector | | | Target | Representative |
|---|---|---|---|---|---|---|---|
| V | nomenclature | names | size | | Guide | polynucleotide | organism |
| V-A | Cas12a | MAD7 | >1000 | aa | crRNA | dsDNA | Francisella cf. |
| | | | | | | | novicida |
| V-B1 | Cas12b1 | c2c1 | >1000 | aa | crRNA, | dsDNA | Alicyclobacillus |
| | | | | | tracrRNA | | acidoterrestris |
| V-B2 | Cas12b2 | — | | | | | Planctomycetes |
| | | | | | | | bacterium |
| | | | | | | | RBG_13_46_10 |
| V-C | Cas12c | c2c3 | >1000 | aa | crRNA, | dsDNA | Oleiphilus spp. |
| | | | | | tracrRNA | | |
| V-D | Cas12d | CasY | >1000 | aa | crRNA | dsDNA | Bacterium |
| | | | | | | | CG09_39_24 |
| V-E | Cas12e | CasX | ~1000 | aa | crRNA, | dsDNA | Deltaproteobacteria |
| | | | | | tracrRNA | | bacterium |
| V-F1 | Cas12f1 | Cas14a, | 400-800 | aa | crRNA, | dsDNA | Uncultured |
| | | c2c10, | | | tracrRNA | | archaeon |
| | | V-U3 | | | | | |
| V-F2 | Cas12f2 | Cas14b | | | crRNA | | Bacillus |
| | | | | | | | thuringiensis |
| | | | | | | | HD-771 |
| V-F3 | Cas12f3 | Cas14c | | | crRNA | | Candidatus |
| | | | | | | | Micrarchaeota |
| | | | | | | | archaeon |
| V-G | Cas12g | — | 700-800 | aa | crRNA, | ssRNA | Hot springs |
| | | | | | tracrRNA | | metagenome |
| V-H | Cas12h | — | ~1000 | aa | crRNA | ssDNA, | Hypersaline lake |
| | | | | | | dsDNA | sediment |
| | | | | | | | metagenome |
| V-I | Cas12i | — | ~1000 | aa | crRNA | ssDNA, | Freshwater |
| | | | | | | dsDNA | metagenome |
| V-J | Cas12j | Casφ | 700-800 | aa | crRNA | dsDNA | Biggiephage |
| | | (Cas- | | | | | |
| | | phi) | | | | | |
| V-K | Cas12K | c2c5 | >700 | aa | crRNA, | no nuclease | Cyanothece spp. |
| | | | | | tracrRNA | activity | PCC 8801 |

| | | | | | | |
|---|---|---|---|---|---|---|
| V-U | — | c2c4, | n.d. | n.d. | n.d. | Gordonia otitidis |

c2c8,
c2c9

Cas12 homologs can be identified using sequence similarity search methods known to those skilled in the art. Typically, a Cas12 protein is capable of interacting with a cognate Cas12 guide to form a Cas12 guide/nucleoprotein complex capable of binding to a target nucleic acid sequence. In some embodiments of the present disclosure, the Cas 12 protein or homolog thereof is a Cas12a protein or homolog thereof.

Cas12a proteins include, but are not limited to, Cas12a from Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA_33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella sp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1).

In Type V systems, nucleic acid target sequence binding typically involves a Cas12 protein and a crRNA, as does the nucleic acid target sequence cleavage. In Type V systems, the RuvC-like nuclease domain of Cas12 protein cleaves both strands of the nucleic acid target sequence in a sequential fashion, see Swarts et al. (Mol. Cell, 2017, 66:221-233), producing 5′ overhangs, which contrasts with the blunt ends generated by Cas9 protein cleavage.

The Cas 12 protein cleavage activity of Type V systems can be independent of a tracrRNA (e.g., Type V-A); and some Type V systems require only a single crRNA that has a stem-loop structure forming an internal duplex. Cas12 protein binds the crRNA in a sequence- and structure-specific manner by recognizing the stem loop and sequences adjacent to the stem loop, most notably the nucleotides 5′ of the spacer sequence, which hybridize to the nucleic acid target sequence. This stem-loop structure is typically in the range of 15 to 22 nucleotides in length. Substitutions that disrupt this stem-loop duplex abolish cleavage activity, whereas other substitutions that do not disrupt the stem-loop duplex do not abolish cleavage activity. Certain Type V systems require the hybridization between a crRNA and tracrRNA, such as the Type V-F1, V-G, V-C, V-E (CasX), V-K, and V-B. See, e.g., Yan et. al. (Science, 2019, 363 (6422): 88-91).

### Cas12 Guides

CRISPR Cas12 chRDNA guides that are capable of forming a nucleoprotein complex with a cognate Cas12 protein, such as a Cas12a protein have been described in the International Patent Application Ser. No. PCT/US21/55394 DNA-containing polynucleotides and guides for CRISPR Type V systems and methods of making and using the same, filed on Oct. 18, 2021. The complexes described therein are capable of targeting a sequence complementary to the spacer sequence.

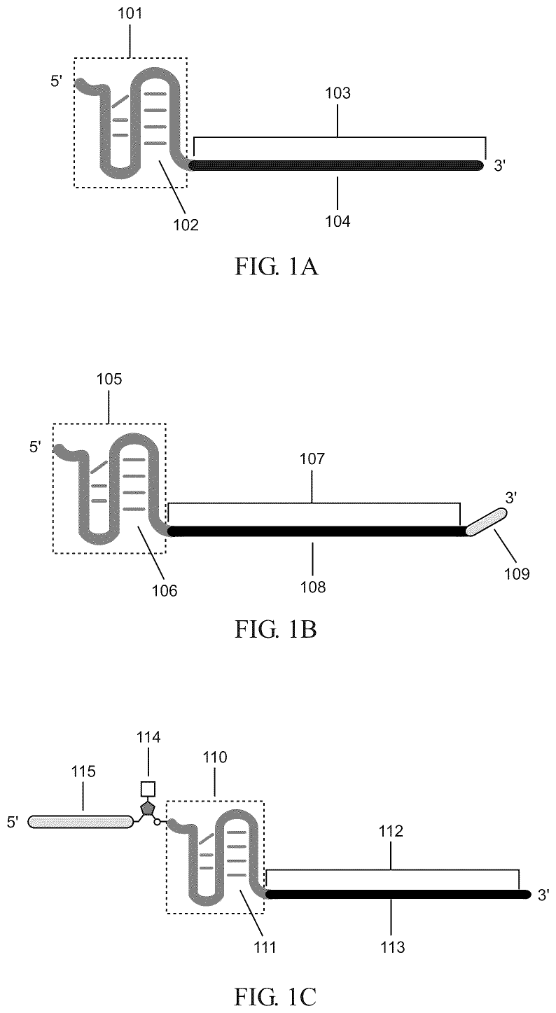

FIG. 1A illustrates an example of an Acidaminococcus spp. BV316 Cas 12a guide molecule comprising the following: an activating region (FIG. 1A, 101), comprising a stem-loop duplex (FIG. 1A, 102); and a spacer sequence (FIG. 1A, 103), comprising a target binding sequence (FIG. 1A, 104). FIG. 1B illustrates an alternative Cas 12a guide molecule comprising the following: an activating region (FIG. 1B, 105), comprising a stem-loop duplex (FIG. 1B, 106); and a spacer sequence (FIG. 1B, 107), comprising a target binding sequence (FIG. 1B, 108) and a 3′ extension (FIG. 1B, 109). The 3′ extension (FIG. 1B, 109) can be connected to the spacer sequence (FIG. 1B, 107) via a linker sequence. FIG. 1C illustrates an alternative Cas12a guide molecule comprising the following: an activating region (FIG. 1C, 110), comprising a stem-loop duplex (FIG. 1C, 111) and a linker nucleotide (FIG. 1C, 114) and a 5′ extension (FIG. 1C, 115); and a spacer sequence (FIG. 1C, 112), comprising a target binding sequence (FIG. 1C, 113).

In Cas12 chRDNA guide molecules of the present disclosure, the targeting region, and also or separately, the activating region, may comprise DNA, RNA, or a mixture of DNA and RNA. In certain embodiments, the targeting and activating region may also comprise other base analogs, modified nucleotides, abasic sites, and the like, as well as synthetic, naturally occurring, and non-naturally occurring modified backbone residues or linkages, or combinations thereof.

In some embodiments, the activating region is between 10-25 bases in length, inclusive of optional abasic sites.

In some embodiments, the targeting region is between 10-30 bases in length, inclusive of optional abasic sites.

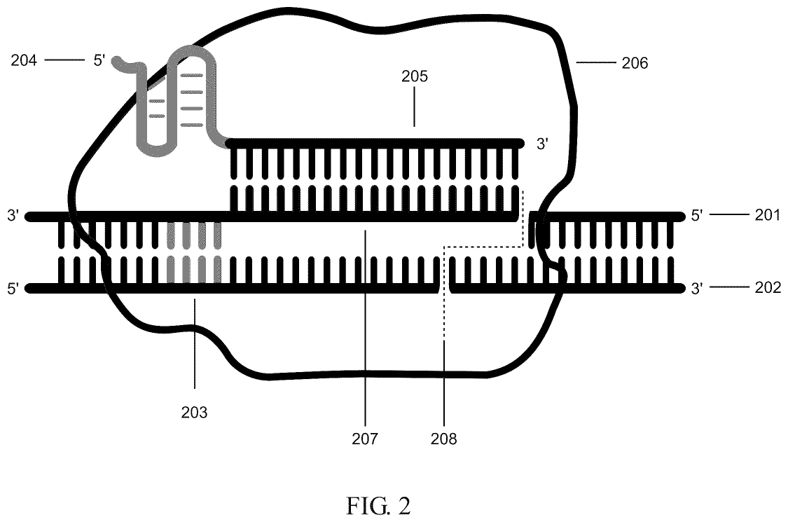

FIG. 2 illustrates a Cas12a protein (FIG. 2, 206) bound to a cognate Cas12a chRDNA guide molecule (FIG. 2, 204) comprising a target binding sequence (FIG. 2, 205). The Cas12a chRDNA guide/nucleoprotein complex unwinds a target polynucleotide comprising the target sequence, and the target binding sequence of the Cas12 chRDNA guide molecule (FIG. 2, 205) is connected via hydrogen bonds (FIG. 2, indicated by a vertical line between polynucleotides) to the target sequence (FIG. 2, 207). In FIG. 2, the target polynucleotide comprises a target strand (FIG. 2, 201) comprising the target sequence (FIG. 2, 207), and a non-target strand (FIG. 2, 202) comprising a PAM sequence (FIG. 2, 203). The PAM sequence (FIG. 2, 203) typically occurs upstream (i.e., in a 5′ direction) of the target sequence (FIG. 2, 207) on the non-target strand (FIG. 2, 202). Formation of hydrogen bonds between the target binding sequence of the Cas12a chRDNA guide molecule (FIG. 2, 205) and the target sequence (FIG. 2, 207) result in the staggered cleavage (FIG. 2, 208) of the target strand (FIG. 2, 201) and the non-target strand (FIG. 2, 202).

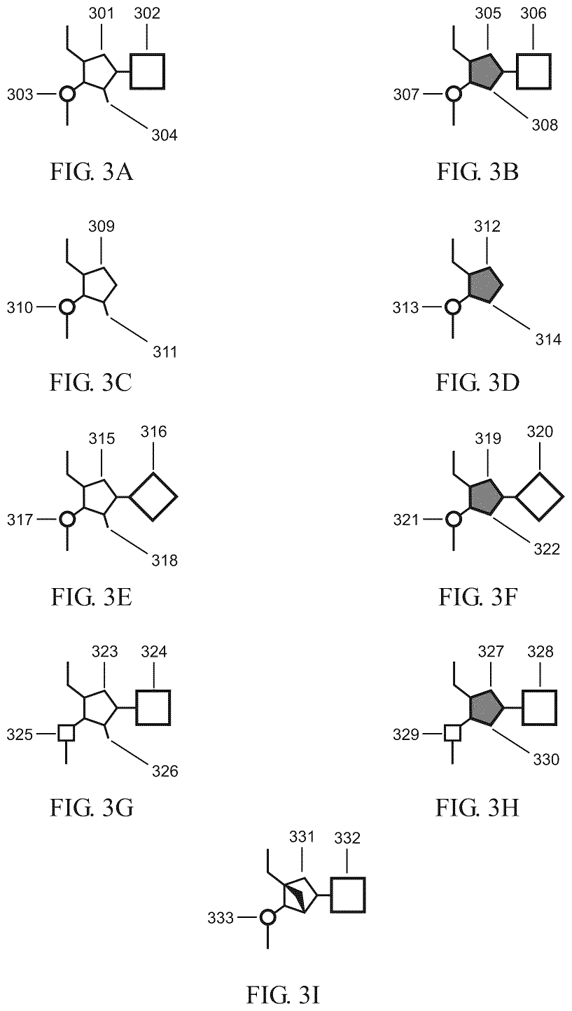

FIG. 3A-FIG. 3I illustrate various canonical and non-canonical nucleotides for use in Cas12 chRDNA guide molecules of the present disclosure. Table 2 presents a series of indicators used in FIG. 3A-FIG. 3I.

TABLE 2
Numerical Indicators of Canonical and Non-canonical
Nucleotides for Use in Cas12 crRNA and chRDNA Guides

| FIG. | Indicator | Description |
|---|---|---|
| FIG. 3A | 301 | a ribose sugar |
| | 302 | a nitrogen base |
| | 303 | a phosphate backbone |
| | 304 | a 2′hydroxyl group |
| FIG. 3B | 305 | a deoxyribose sugar |
| | 306 | a nitrogen base |
| | 307 | a phosphate backbone |
| | 308 | a lack of a 2′hydroxyl group |
| FIG. 3C | 309 | a ribose sugar |
| | 310 | a phosphate backbone |
| | 311 | a 2′hydroxyl group |
| FIG. 3D | 312 | a deoxyribose sugar |
| | 313 | a phosphate backbone |
| | 314 | a lack of a 2′hydroxyl group |
| FIG. 3E | 315 | a ribose sugar |
| | 316 | a non-canonical nitrogen base or base mimic |
| | 317 | a phosphate backbone |
| | 318 | a 2′hydroxyl group |
| FIG. 3F | 319 | a deoxyribose sugar |
| | 320 | a non-canonical base or base mimic |
| | 321 | a phosphate backbone |
| | 322 | a lack of a 2′hydroxyl group |
| FIG. 3G | 323 | a ribose sugar |
| | 324 | a nitrogen base |
| | 325 | a chemically modified backbone |
| | 326 | a 2′hydroxyl group |
| FIG. 3H | 327 | a deoxyribose sugar |
| | 328 | a nitrogen base |
| | 329 | a chemically modified backbone |
| | 330 | a lack of a 2′hydroxyl group |
| FIG. 3I | 331 | a non-canonical or chemically modified sugar |
| | 332 | a nitrogen base |
| | 333 | a phosphate backbone |

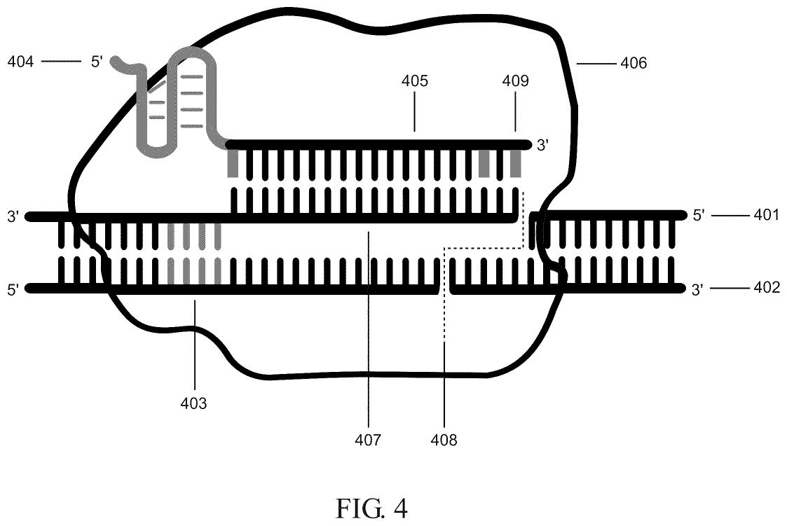

FIG. 4 illustrates a Cas12a protein (FIG. 4, 406) bound to a cognate Cas12a chRDNA guide molecule (FIG. 4, 404) comprising a target binding sequence (FIG. 4, 405), wherein the target binding sequence (FIG. 4, 405) comprises non-RNA nucleotides (FIG. 4, 409) such as a canonical and non-canonical nucleotide presented in FIG. 3B-FIG. 31. The Cas12a chRDNA guide/nucleoprotein complex unwinds a target polynucleotide comprising the target sequence, and the target binding sequence of the Cas12 chRDNA guide molecule (FIG. 4, 405) is connected via hydrogen bonds (FIG. 4, indicated by a vertical line between polynucleotides) to the target sequence (FIG. 4, 407). In FIG. 4, the target polynucleotide comprises a target strand (FIG. 4, 401) comprising the target sequence (FIG. 4, 407), and a non-target strand (FIG. 4, 402) comprising a PAM sequence (FIG. 4, 403). The PAM sequence (FIG. 4, 403) typically occurs upstream (i.e., in a 5′ direction) of the target sequence (FIG. 4, 407) on the non-target strand (FIG. 4, 402). Formation of hydrogen bonds between the target binding sequence of the chRDNA guide molecule (FIG. 4, 405) and the target sequence (FIG. 4, 407) result in the staggered cleavage (FIG. 4, 408) of the target strand (FIG. 4, 401) and the non-target strand (FIG. 4, 402).

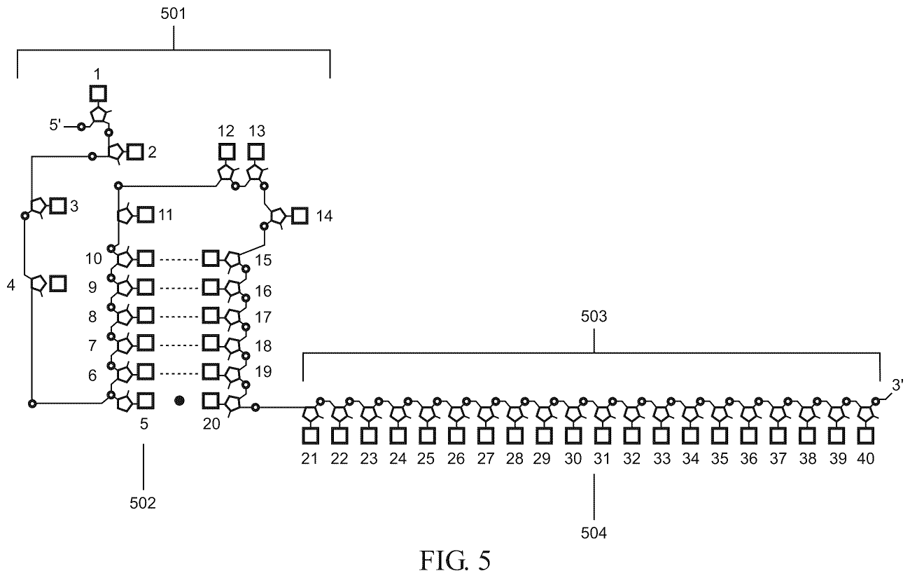

FIG. 5 illustrates an example of an Acidaminococcus spp. (strain BV3L6) Cas12a crRNA guide molecule comprising the following: an activating region (FIG. 5, 501), comprising a stem-loop duplex (FIG. 5, 502); and a spacer (FIG. 5, 503), comprising a target binding sequence (FIG. 5, 504). Each nucleotide position in the activating region (FIG. 5, 501) and in the spacer (FIG. 5, 503) is labeled starting at the 5′ end of the guide molecule, wherein the activating region and the target binding region each comprises RNA.

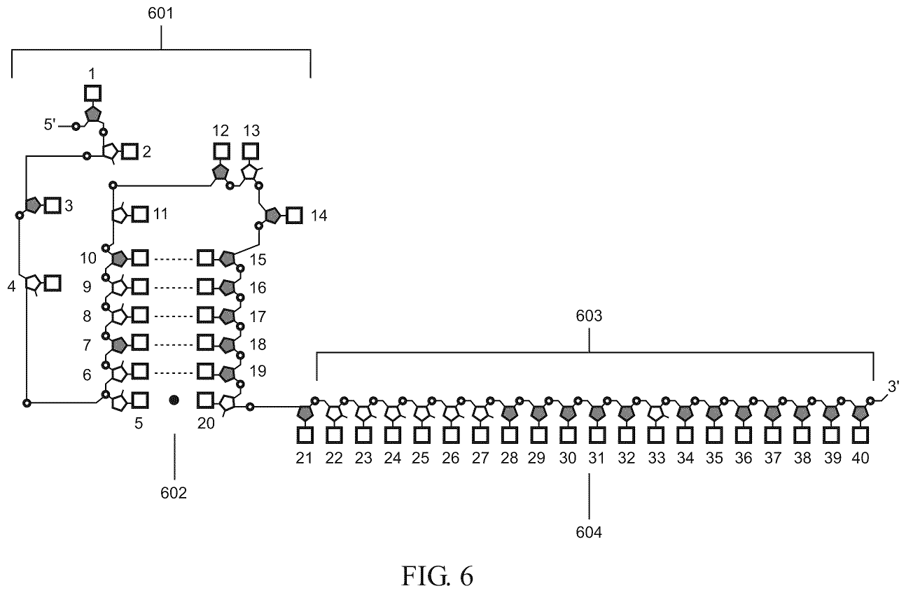

FIG. 6 illustrates an example of an Acidaminococcus spp. (strain BV3L6) Cas12a chRDNA guide molecule comprising the following: an activating region (FIG. 6, 601), comprising a stem-loop duplex (FIG. 6, 602); and a spacer (FIG. 6, 603), comprising a target binding sequence (FIG. 6, 604). Each nucleotide position in the activating region (FIG. 6, 601) in the spacer (FIG. 6, 603) is labeled starting at the 5′ end of the guide molecule, wherein the activating region comprises a mixture of RNA (white fill) and DNA (grey fill) and the target binding sequence comprises a mixture of RNA (white fill) and DNA (grey fill).

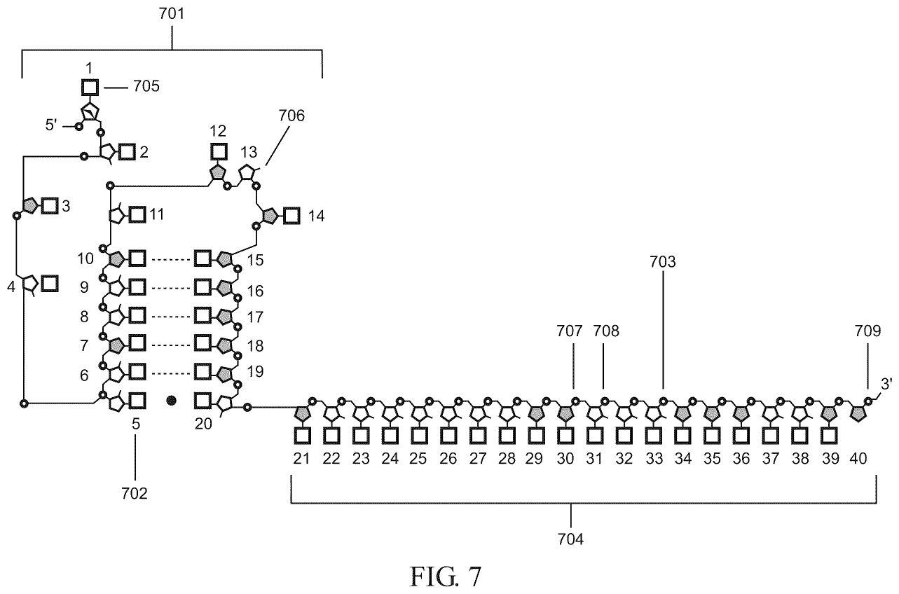

FIG. 7 illustrates an example of an Acidaminococcus spp. (strain BV3L6) Cas12a chRDNA guide molecule comprising the following: an activating region (FIG. 7, 701), comprising a stem-loop duplex (FIG. 7, 702), and a spacer (FIG. 7, 703), comprising a target binding sequence (FIG. 7, 704). Each nucleotide position in the activating region (FIG. 7, 701) and in the spacer (FIG. 7, 703) is labeled starting at the 5′ end of the guide molecule, wherein the activating region comprises a mixture of RNA (white fill) and DNA (grey fill). The Cas12a chRDNA guide molecule further comprises other non-canonical nucleotides, such as a chemically modified sugar nucleotide (FIG. 7, 705), an abasic ribonucleotide (FIG. 7, 706), a deoxy-ribonucleotide with a chemically modified backbone (FIG. 7, 707), a ribonucleotide with a chemically modified backbone (FIG. 7, 708), and an abasic deoxy-ribonucleotide (FIG. 7, 709).

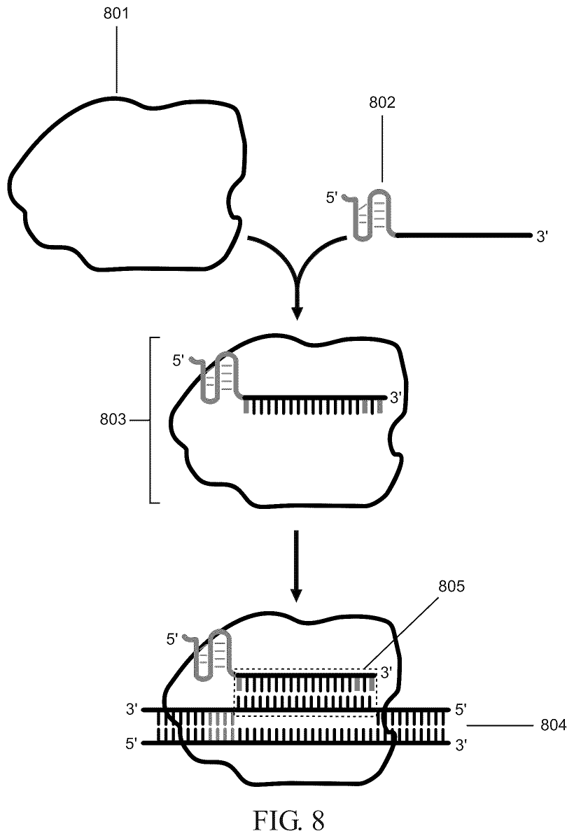

FIG. 8 illustrates the formation of a Cas12 chRDNA guide/nucleoprotein complex, wherein a Cas 12 protein (FIG. 8, 801) binds a Cas12 chRDNA guide molecule (FIG. 8, 802) to form a Cas12 chRDNA guide/nucleoprotein complex (FIG. 8, 803). The Cas12 chRDNA guide/nucleoprotein complex (FIG. 8, 803) binds a target polynucleotide (FIG. 8, 804), wherein the target polynucleotide contains a target sequence complementary to the target binding sequence of the Cas12 chRDNA guide molecule, and hydrogen bonds form between the target binding sequence of the Cas12 chRDNA guide molecule and the target sequence (FIG. 8, 805).

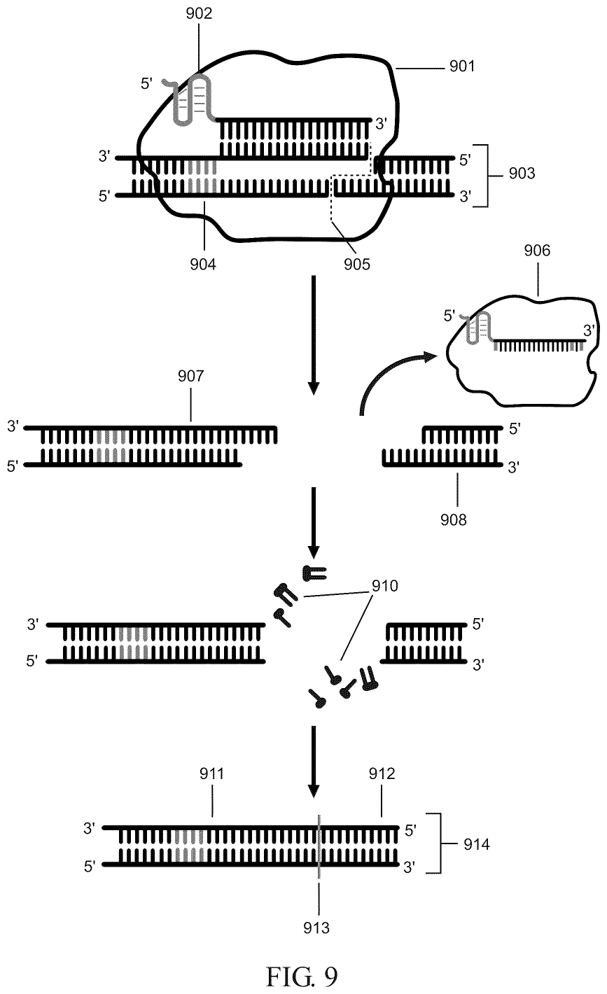

FIG. 9 illustrates the generation of insertion or deletion (indels) in a target polynucleotide by a Cas12 chRDNA guide/nucleoprotein complex, wherein a Cas12 protein (FIG. 9, 901) complexed with a Cas12 chRDNA guide molecule (FIG. 9, 902) binds a target polynucleotide (FIG. 9, 903) comprising a PAM (FIG. 9, 904), and the target polynucleotide is cleaved (FIG. 9, 905) by the Cas12 chRDNA guide/nucleoprotein complex. After targeting has occurred, the Cas12 chRDNA guide/nucleoprotein complex disassociates from the target polynucleotide (FIG. 9, 906), wherein the target polynucleotide comprises an upstream (i.e., in a 5′ direction) strand (FIG. 9, 907) and a downstream (i.e., in a 3′ direction) strand (FIG. 9, 908) relative to the PAM (FIG. 9, 904). The cellular DNA repair machinery repairs the target polynucleotide through insertion or deletion (FIG. 9, 910) of the sequence around the cleavage site in the target polynucleotide. The upstream strand (FIG. 9, 911) and a downstream strand (FIG. 9, 912) are rejoined and the edited target polynucleotide (FIG. 9, 914) comprises indels (FIG. 9, 913) at the cleavage site, wherein the edited target polynucleotide has a different sequence relative to an unedited target polynucleotide. In some embodiments, the generation of insertion or deletion (indels) in a target polynucleotide by a Cas12 chRDNA guide/nucleoprotein complex occurs inside a cell.

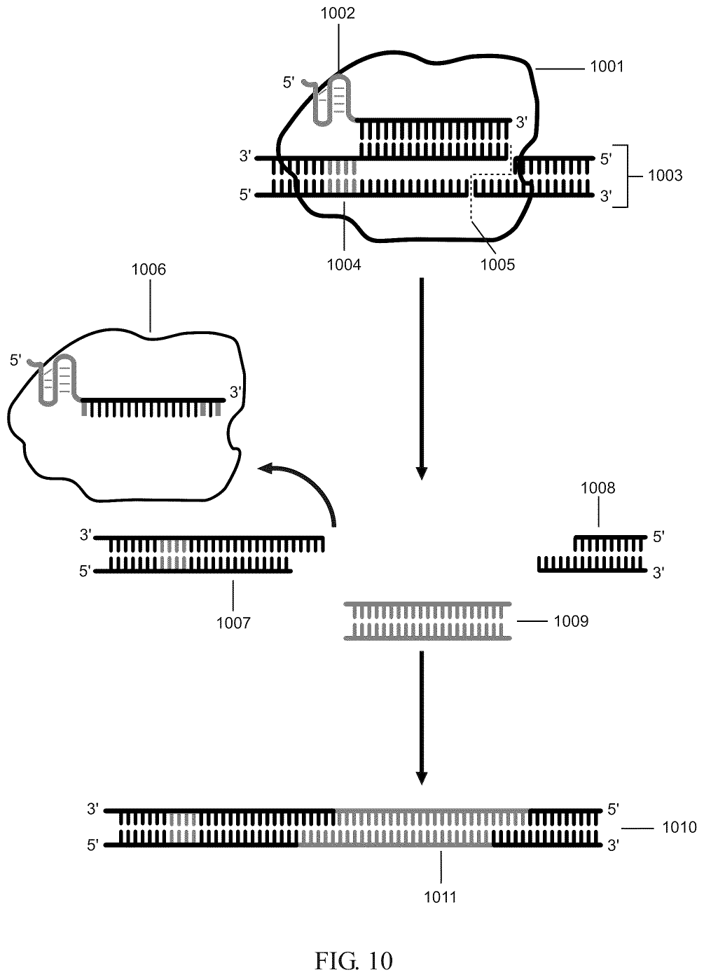

FIG. 10 illustrates incorporation of a donor polynucleotide sequence into a target polynucleotide, wherein a Cas12 protein (FIG. 10, 1001) complexed with a Cas12 chRDNA guide molecule (FIG. 10, 1002) binds a target polynucleotide (FIG. 10, 1003) comprising a PAM (FIG. 10, 1004), and the target polynucleotide is cleaved (FIG. 10, 1005) by the Cas12 chRDNA guide/nucleoprotein complex. After targeting has occurred, the Cas12 chRDNA guide/nucleoprotein complex disassociates from the target polynucleotide (FIG. 10, 1006), wherein the target polynucleotide comprises an upstream (i.e., in a 5′ direction) strand (FIG. 10, 1007) and a downstream (i.e., in a 3′ direction) strand (FIG. 10, 1008) relative to the PAM (FIG. 10, 1004), and wherein a donor polynucleotide is provided (FIG. 10, 1009). The cellular DNA repair machinery repairs the target polynucleotide (FIG. 10, 1010) using the donor polynucleotide (FIG. 10, 1011). The resulting edited target polynucleotide (FIG. 10, 1010) comprises the donor sequence (FIG. 10, 1011) at the target site. In some embodiments, the incorporation of a donor polynucleotide sequence into a target polynucleotide occurs inside a cell.

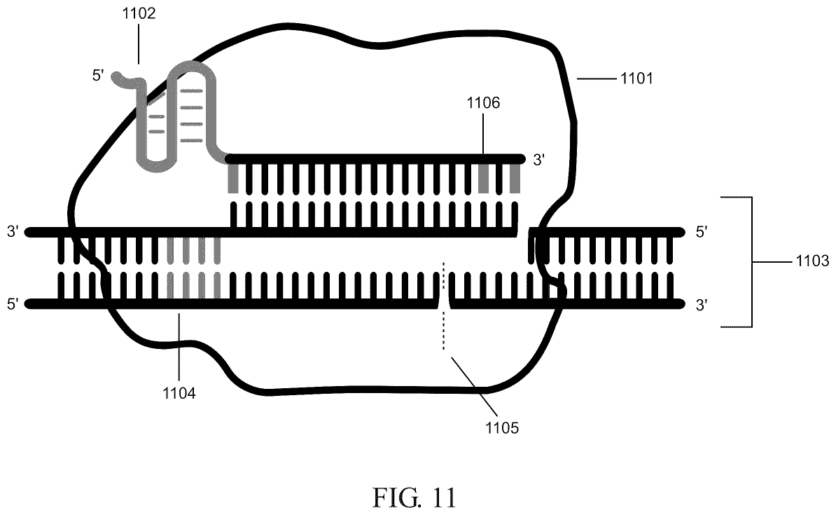

FIG. 11 illustrates nicking of a target polynucleotide, wherein a Cas12 protein (FIG. 11, 1101) complexed to a Cas12 chRDNA guide molecule (FIG. 11, 1102), comprising DNA bases in the target binding sequence (FIG. 11, 1106), binds a target polynucleotide (FIG. 11, 1103) comprising a PAM (FIG. 11, 1104), and the target polynucleotide is nicked (FIG. 11, 1105) in only one strand of the target polynucleotide by the Cas12 chRDNA guide/nucleoprotein complex.

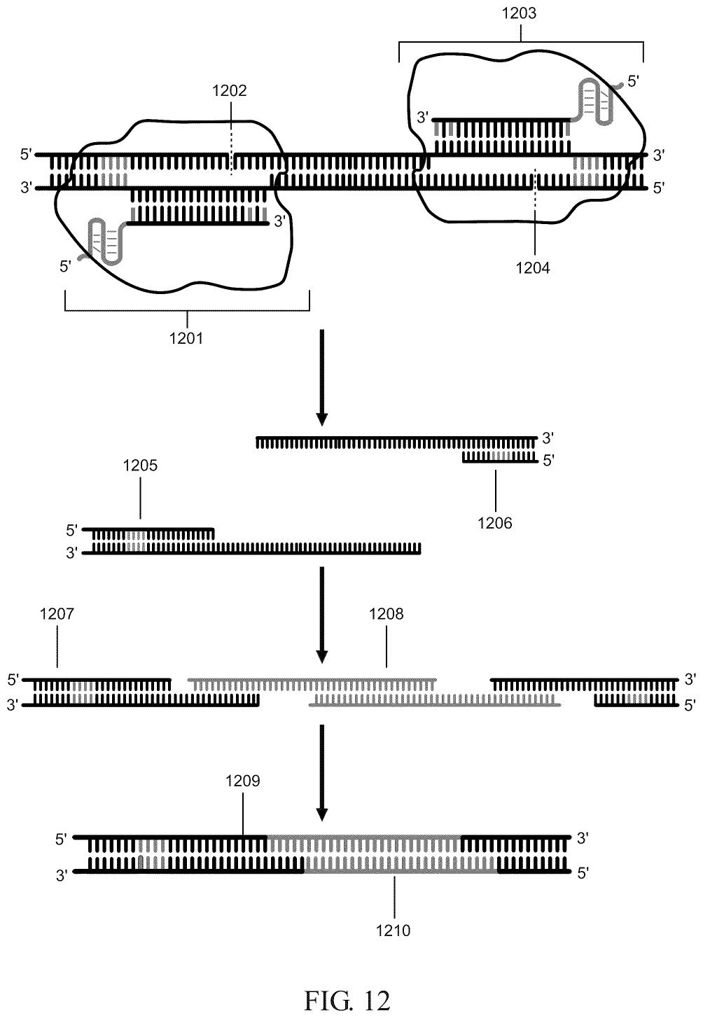

FIG. 12 illustrates the use of two nicking Cas12 chRDNA guide/nucleoprotein complexes to generate a staggered double-strand break in a target polynucleotide, wherein a first Cas12 chRDNA guide/nucleoprotein complex binds an upstream (i.e., in a 5′ direction) target sequence of a target polynucleotide (FIG. 12, 1201) creating a first nick in the target polynucleotide (FIG. 12, 1202) and a second Cas12 chRDNA guide/nucleoprotein complex binds a downstream (i.e., in a 3′ direction) target sequence of a target polynucleotide (FIG. 12, 1203) creating a second nick in the target polynucleotide (FIG. 12, 1204). After tandem nicking has occurred, the post-cleavage target polynucleotide comprises an upstream (i.e., in a 5′ direction) strand (FIG. 12, 1205) and a downstream (i.e., in a 3′ direction) strand (FIG. 12, 1206) with 5′ overhangs. A donor polynucleotide is provided, and the cellular DNA repair machinery repairs the target polynucleotide (FIG. 12, 1207) using the donor polynucleotide (FIG. 12, 1208). The resulting edited target polynucleotide (FIG. 12, 1209) comprises the donor sequence (FIG. 12, 1210) at the tandem nicked site. In some embodiments, the use of two nicking Cas12chRDNA guide/nucleoprotein complexes to generate a staggered DSB in the target polynucleotide occurs inside a cell.

Methods of designing particular Cas12 chRDNA guide molecules into which deoxyribonucleotides, and optionally additional modifications (such as base analogs, modified nucleotides, abasic sites, modified backbone residues or linkages, or combinations thereof) can be designed as described e.g., in the Ser. No. PCT/US21/55394. Briefly, to design a Cas12 guide, the genomic sequence for the gene to be targeted is first identified. The exact region of the selected gene to target will depend on the specific application. For example, in order to activate or repress a target gene, Cas12 can be targeted to the promoter driving expression of the gene or to the 5′ constitutively expressed exons, to reduce the chance of removal of the targeted region from mRNA due to alternative splicing. Other exons in the N-terminal portion can be targeted because frameshift mutations here will result in a non-functional protein product. Exons that code for essential protein domains can also be targeted. For gene editing using HDR, the target sequence should be close to the location of the desired edit. In this case, the location where the edit is desired is identified and a target sequence is selected nearby.

In some embodiments, a Cas12 chRDNA guide can be designed to bind outside of the cleavage site of the Cas 12 protein so that the target nucleic acid can be released from the Cas12 nucleoprotein complex. In some embodiments, a Cas12 chRDNA guide can be designed to bind inside of the cleavage site of the Cas 12 protein. In this case, the target nucleic acid can will be bound to the Cas12 nucleoprotein complex.

In some embodiments of the Cas12 chRDNA guide molecule, the targeting region, the activating region, or both contain deoxyribonucleotides or modified nucleotides. In some embodiments, the Cas12a chRDNA guide comprises one or more, e.g., 23 or less deoxyribonucleotides. In some embodiments, all of the deoxyribonucleotides in the targeting region of the chRDNA form canonical base pairs with the target sequence. In some embodiments, at least one of the deoxyribonucleotides in the targeting region of the chRDNA does not form a canonical base pair with the target sequence, or forms a non-canonical base pair with the target sequence.

### Cas12 Proteins

Cas 12 proteins of the present disclosure include, but are not limited to, Cas12 wild type proteins derived from Type V CRISPR-Cas systems, modified Cas12 proteins, variants of Cas 12 proteins, Cas12 orthologs, and combinations thereof. In some embodiments, the Cas12 protein is a wild type Cas12a protein, a modified Cas12a protein, a variant of a Cas 12a protein, a Cas 12a ortholog, or a combination thereof.

Cas12 proteins typically consist of six domains corresponding to the RECI, REC2, PAM interacting (PI), Nuclease (Nuc), Wedge (WED), and RuvC domains. See, e.g., Yamano et al. (Cell, 2016, 165 (4): 949-962). The WED domain and RuvC domain can have a tripartite sequence architecture, interrupted by sequences from other domains. For example, the Acidaminococcus spp. Cas12a WED domain sequence is interrupted by the RECI, REC2, and PI domain sequences. Additionally, certain subtypes of Cas12 proteins contain a bridge helix domain that occurs adjacent to, or between, the RuvC domain sequences.

To create a modified Cas12 protein, regions of the Cas12 protein can be modified to modulate the activity of the Cas12 protein. For example, regions of the Acidaminococcus spp. (strain BV3L6) Cas12a protein corresponding to residues of the PI domain (598-718) and WED domain (526-597 and 719-883) can be modified to alter PAM specificity. See, e.g., Tóth et al. (Nucleic Acid Research, 2020, 48 (7): 3722-3733). The region in the Acidaminococcus spp. (strain BV3L6) Cas12a protein corresponding to residues of the REC1 (24-319) and REC2 (320-526) domains can be modified to alter target engagement and cleavage kinetics. Regions of the REC1 (226-304) and REC2 (368-435) domains interact directly with the PAM distal end of the target binding sequence and target sequence, and can be engineered to modify efficiency of target sequence cleavage. Regions of the Nuc domain (1066-1261) and RuvC domain (940-956, 957-1065, and 1261-1307) can be modified to alter the cleavage efficiencies of the target strand, non-target strand, or target strand and non-target strand, of the target sequence. Engineering these regions can comprise introducing mutations, replacing with corresponding regions from other Cas12 orthologues, deletions, insertions, etc.

Modified Cas 12 proteins can be used in combination with Cas 12 chRDNA guide molecules to alter the activity or specificity of the Cas12 protein. In some instances, a Cas12 protein can be modified to provide enhanced activity or specificity when complexed with a Cas12 chRDNA guide molecule, wherein the Cas12 modifications occur in the RECI, REC2, RuvC, WED, and/or Nuc domain(s). In some instances, a Cas12 protein can be modified to provide enhanced activity or specificity when complexed with a Cas12 chRDNA guide molecule, wherein the Cas12a modifications occur in regions 226-304, 368-435, 940-956, 978-1158, 1159-1180, and 1181-1298 (numbering based on the Acidominococus spp. Cas12a sequence).

In some embodiments, the Cas 12 protein is an nCas 12 protein, that is nuclease-deficient variant, also termed a “nicking Cas12” or “Cas12-nickase.” Such molecules lack a portion of the endonuclease activity and therefore can only nick one strand of the target nucleic acid. See, e.g., Jinck et al. (Science, 2012, 337:816-821). This may be accomplished, for example, by introducing mutation(s) into the RuvC nuclease domain. Non-limiting examples of such modifications can include D917A, E1006A, and D1225A, to the RuvC nuclease domain of the F. novicida Cas 12a protein. It is understood that the mutation of other catalytic residues to reduce activity of the RuvC nuclease domain can also be carried out by those skilled in the art. The resultant nCas12 protein is unable to cleave double-stranded DNA, but retains the ability to complex with a guide molecule, bind a target DNA sequence, and nick only one strand of the target DNA. Targeting specificity is determined by Cas12 protein binding to the PAM sequence, and by complementary base pairing of guide molecule to the genomic locus. In some embodiments of the present disclosure, the nCas 12 protein is an nCas 12a protein.

In some embodiments, the Cas 12 protein is a dCas 12 protein, that is a nuclease-deactivated variant, also termed a “catalytically inactive Cas12 protein,” an “enzymatically inactive Cas12,” a “catalytically dead Cas12,” or a “dead Cas12.” Such molecules lack endonuclease activity, and can therefore be used to regulate genes in an RNA-guided manner. See, e.g., Jinek et al. (Science, 2012, 337:816-821). Mutations of catalytic residues to eliminate activity of the RuvC domain can be carried out by those skilled in the art. The resultant dCas12 protein is unable to cleave double-stranded DNA, but retains the ability to complex with a guide molecule and bind a target DNA sequence. Targeting specificity is determined by Cas 12 protein binding to the PAM sequence, and by complementary base pairing of guide molecule to the genomic locus. In some embodiments of the present disclosure, the dCas 12 protein is a dCas 12a protein.

Certain Cas 12 protein subtypes lack nuclease activity, due to either inactivation of the RuvC-like nuclease domain, or absences in part or in whole of the RuvC-like nuclease domain. One such subtype, Type V-K and associated protein Cas12k, instead are associated with Tn7-like transposable elements tnsB, tnsC, tniQ. See, e.g., Strecker et al. (Science, 2019, 364 (6448): 48-53). Cas12k retains the ability to complex with a guide molecule, and to bind a target DNA sequence, and the associated Tn7-like proteins facilitate the RNA-guided transposition of DNA sequences. In some embodiments of the present disclosure, the Cas12 chRDNA guide/nucleoprotein complex is a Cas12k chRDNA guide/nucleoprotein complex.

Other amino acid alterations may include amino acids with glycosylated forms, aggregative conjugates with other molecules, and covalent conjugates with unrelated chemical moieties (e.g., pegylated molecules). Covalent variants can be prepared by linking functionalities to groups that are found in the amino acid chain or at the N- or C-terminal residue. In some cases, mutated site-directed polypeptides may also include allelic variants and species variants.

In certain embodiments, the Cas 12 protein may be a fusion or chimeric protein containing a first domain from a Cas12 protein, and a second domain from a different protein, such as a Csy4 protein. The fusion modification to a Cas12 protein may confer additional activity on the modified Cas12 protein. Such activities can include nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, reverse transcriptase activity, deubiquitinating activity, adenylation activity, deadenylation activity, SUMOylating activity, deSUMOylating activity, ribosylation activity, deribosylation activity, and/or myristoylation activity or demyristoylation activity that modifies a polypeptide associated with nucleic acid target sequence (e.g., a histone).

In certain embodiments, a Cas12 protein may contain one or more NLS sequences (e.g., appended to, and/or inserted within, the Cas12 protein sequence). An NLS sequence may be located, for example, at the N-terminus, the C-terminus, or internally within a Cas12 protein (such as a Cas12a protein), including combinations thereof (e.g., one or more NLS at the N-terminus and one or more NLS at the C-terminus). The NLS sequence may be derived from SV40 large T-antigen, nucleoplasmin, 53BP1, VACM-1/CUL5, CXCR4, VP1, ING4, IER5, ERK5, UL79, EWS, Hrp1, cMyc (1), cMyc (2), Mouse c-able IV, Mata2, and MINIYO.

NLS sequences can be covalently attached (e.g., to a Cas12 protein, to another NLS sequence(s), or to a fusion peptide sequence attached to a Cas 12 protein) either directly or via a linker polypeptide. The length of a linker sequence can be optimized depending on the structural characteristics of the particular Cas12 protein (e.g., solvent accessibility of the termini, the presence of other critical functional peptide sequences at the termini, etc.) to ensure the accessibility of the NLS sequence for cognate importin protein binding and trafficking.

In some embodiments, a linker sequence contains at least one glycine, serine, and/or threonine residue. In some embodiments, a linker sequence contains at least one glycine residue and at least one serine residue. In some embodiments, a linker sequence contains a plurality of glycine residues and at least one serine residue. In some embodiments, a linker sequence consists of or comprises a GS sequence.

### Genomic Editing of Cells Using Cas12-chRDNA Guide Nucleoprotein Complexes

Delivery of Cas12 chRDNA guide molecules, Cas12 proteins, and Cas12-chRDNA guide nucleoprotein complexes of the present disclosure to cells, in vitro, ex vivo, or in vivo, may be achieved by a number of methods known to one of ordinary skill in the art. Non-limiting methods to introduce these components into a cell include viral vector delivery, sonoporation, cell squeezing, electroporation, nucleofection, lipofection, particle gun technology, microprojectile bombardment, or chemicals (e.g., cell penetrating peptides).

In some embodiments, electroporation can be used to deliver the Cas12 chRDNA guide molecules of the present disclosure to cells. Electroporation may also be used to deliver Cas12 chRDNA guide/nucleoprotein complexes of the present disclosure. In these methods, the chRDNA guide molecules, or the Cas12 chRDNA guide/nucleoprotein complexes, are mixed in an electroporation buffer with the target cells to form a suspension. This suspension is then subjected to an electrical pulse at an optimized voltage, which creates temporary pores in the phospholipid bilayer of the cell membrane, permitting charged molecules (like nucleic acids and proteins) to be driven through the pores and into the cell. Reagents and equipment to perform electroporation are sold commercially.

In some embodiments, the delivery of Cas12 chRDNA guides, Cas12 proteins, and Cas12-chRDNA guide nucleoprotein complexes is achieved by packaging the components into a compartment. The compartment comprising the components can be administered in vivo (e.g., in cells of a living organism, with the proviso that, in some embodiments, the organism is a non-human organism). In some embodiments, the compartment is a biological compartment such as a virus (lentivirus, adenovirus) or a liposome. In some embodiments, the compartment is a non-biological compartment selected from nanospheres, liposomes, quantum dots, nanoparticles, microparticles, nanocapsules, vesicles, polyethylene glycol particles, hydrogels, and micelles.

In some embodiments, the compartment is a lipid nanoparticle (LNP)

Cas12-chRDNA guide nucleoprotein complexes can be used to cleave or bind to a target nucleic acid when a Cas12 chRDNA guide molecule can be introduced into cells with a Cas12 protein, thereby forming a Cas12-chRDNA guide nucleoprotein complex. The nucleoprotein complex can hybridize to a target nucleic acid comprising a PAM. The nucleoprotein complex comprises a Cas12 chRDNA guide having a targeting region complementary to a nucleic acid target sequence. Optionally a second Cas12-chRDNA guide nucleoprotein complex comprises a Cas12 chRDNA guide having a second targeting region complementary to a second nucleic acid target is also introduced into a cell.

The steps of binding a nucleic acid target sequence can be carried out in vitro (e.g., in a biochemical reaction or in cultured cells); in vivo (e.g., in cells of a living organism or a patient); or ex vivo (e.g., cells removed from a subject or patient for return to the subject or patient).

In an additional embodiment, a donor polynucleotide can also be introduced into a cell to facilitate incorporation of at least a portion of the donor polynucleotide into genomic DNA of the cell using the Cas 12 protein-chRDNA guide nucleoprotein complex. Typically, a donor polynucleotide is brought into close proximity to a site-directed target nucleic acid break by binding it to the Cas12 protein that generates the double-strand break (e.g., Cas12a). The proximity enhances insertion (e.g., homologous recombination) of the donor polynucleotide into the site of the double-strand break.

### Therapeutic Compositions, Applications, and Methods

Cas12 chRDNA guide molecules, and Cas12-chRDNA guide nucleoprotein complexes of the present disclosure can be used in the production of modified cells used for therapeutic purposes. As disclosed in the Ser. No. PCT/US21/55394, the modified cells can be used for adoptive cell therapy such as adoptive immunotherapy.

Lymphocytes can be isolated from a subject, such as a human subject, for example from blood or from solid tumors, such as in the case of TILs, or from lymphoid organs such as the thymus, bone marrow, lymph nodes, and mucosal-associated lymphoid tissues by techniques well known in the art. Upon isolation, lymphocytes can be characterized in terms of specificity, frequency, and function, e.g., by the ELISPOT assay, which measures the frequency of T cell response. The isolated lymphocytes can optionally be activated using techniques well known in the art in order to promote proliferation and differentiation into specialized effector lymphocytes.

In some embodiments, the isolated lymphocytes can be modified using Cas12-chRDNA guide nucleoprotein complexes of the present disclosure e.g., by inserting a gene encoding a chimeric antigen receptor (CAR). In some embodiments, Cas12-chRDNA guide nucleoprotein complexes are used to inactivate the endogenous T-cell receptor e.g., TRAC gene. The resulting lymphocytes form CAR-T cells or CAR-NK cells for use in adoptive immunotherapies.

Additionally, the Cas12-chRDNA nucleoprotein complexes can be used to armor adoptive cells for survival in the host. In some embodiments, the armoring modification comprises inactivation of an immune checkpoint protein such as PD-1 protein encoded by the PDCD1 gene. Other immune checkpoint proteins that can be inactivated include Cytotoxic T-Lymphocyte Antigen 4 (CTLA-4, also known as CD152), LAG3 (also known as CD223), Tim3 (also known as HAVCR2), BTLA (also known as CD272), BY55 (also known as CD160), TIGIT (also known as IVSTM3), LAIR1 (also known as CD305), SIGLEC10, 2B4 (also known as CD244), PPP2CA, PPP2CB, PTPN6, PTPN22, CD96, CRTAM, SIGLEC7, SIGLEC9, TNFRSF10B, TNFRSF10A, CASP8, CASPIO, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, and GUCY1B3. In some embodiments, one or more immune checkpoint molecules are inactivated using Cas12-chRDNA guide nucleoprotein complexes.

In some embodiments, the armoring modification comprises inactivation of Beta-2 microglobulin (B2M), a component of MHC class I molecules present on nucleated cells. In some embodiments, the armoring modification comprises inserting the HLA-E gene into the inactivated B2M locus.

In some embodiments, the invention is a method of treating or alleviating diseases or conditions by administering cells that have been modified with Type V CRISPR system comprising chRDNA of the present disclosure. Table 3 lists the diseases or conditions with genes to be targeted with Type V CRISPR system comprising chRDNA.

TABLE 3
Diseases and conditions and genes targeted
by Type V CRISPR - chRDNA technology

| | |
|---|---|
| Disease or condition | Gene target |

LNP technology targeting the liver

| | |
|---|---|
| Non-alcoholic steatohepatitis | HSD17B13, DGAT2, PNPLA3, HNF |
| | gene family members (HNF1, HNF4) |
| Alpha-1-antitrypsin deficiency | SERPIN1A |
| Hepatitis B virus infection | HBV |
| Transthyretin amyloidosis | TTR |
| Cardiovascular disease | Lp(a), ANGPTL3, APOC3, PCSK9, |
| | AGT |
| Idiopathic pulmonary fibrosis | TERT-hTR |
| Pulmonary arterial hypertension | BMPR2 |
| Acute hepatic porphyria | ALAS1 |
| Glycogen storage diseases | GSD1a |
| Hyperoxaluria, Primary | HAO1 (glycolate oxidase), |
| Hyperoxaluria | LDHA |
| Gout | XDH |
| Hemophilia | Antithrombin |
| Hemophilia A | Factor VIII |
| Hemophilia B | Factor IX |
| Factor VII deficiency | Factor VII |
| Factor XIII deficiency | Factor XIII |
| Factor X deficiency | Factor X |
| Hunter syndrome | Iduronate sulfatase |
| Complement-mediated diseases | C5, C3, CFB |
| Alcohol abuse | ALDH2 |
| Hereditary angioedema | KLKB1 (kallikrein B1), F12, C1 |
| | esterase inhibitor |
| Usher syndrome 2A | USH2A |
| Ornithine transcarbamylase deficiency | OT |
| Hereditary hemochromatosis | HFE |
| Mucopolysaccharidosis type 1 (MPS1) | Alpha-L-iduronidase |

LNP technology targeting eyes

| | |
|---|---|
| Leber congenital amaurosis 10 | CEP290 |
| Retinitis pigmentosa 4 | RHO |

TABLE 4
iPSC technology

| Disease or condition | Gene target |
|---|---|
| Cardiac fibrosis | FAP (fibroblast activation |
| | protein alpha) |
| Spinocerebellar ataxia type 1 | ATXN1 |
| Spinal Muscular Atrophy (SMA) | SMN1 |

TABLE 5
Cell therapies for cardiovascular and neurological diseases

| Disease or condition | Genetically modified cells |
|---|---|
| Heart failure | HLA⁻CIITA⁻ cardiomyocytes |
| Obstructive hypertrophic | HLA⁻CIITA⁻ cardiac myosin⁻ |
| cardiomyopathy (oHCM) | cardiomyocytes |
| Amyotrophic lateral | HLA⁻CIITA⁻ motor neurons |
| sclerosis (ALS) | |
| Spinal injury | HLA⁻CIITA⁻ neurons |
| Spinal Muscular Atrophy (SMA) | HLA⁻CIITA⁻ neurons |
| Spinal injury | HLA⁻CIITA⁻ neurons |

TABLE 6
Xenotransplantation

| Animal | Genetic modification |
|---|---|
| Pig | Gene inactivation: |
| | GGTA1, b4GalNT2, CMAH, GT |
| | (alpha(1,3)-galactosyltransferase), GHR, |
| | one or more of Class I SLA |
| | Gene insertion: |
| | A20, HO-1, FAT-1, TNF-alpha receptor, |
| | CD39, hirudin, TFPI, EPCR, TBM, CD46, |
| | DAF (CD55), CD59, CR1, CTLA4, CD47, |
| | one or more of Class I HLA |

In some embodiments, the Type V CRISPR system comprising chRDNA produces a genome modification resulting in expression or elimination of one or more genes listed in Table 3 in the modified cell. In some embodiments, the aberrant expression of the gene results in a disease or condition listed in Table 3 and elimination or expression of the gene resulting from the genome modification alleviates the diseases or condition.

In some embodiments, an exogenous nucleic acid is inserted into the genome of a cell. According to the instant disclosure (see FIG. 10 and FIG. 12), a donor polynucleotide comprising a copy of one or more exogenous gene is provided. The exogenous gene comprises a sequence coding for the protein under the control of the gene's promoter, another promoter active in the target cell, or a constitutive promoter. The exogenous gene is selected from the list provided in Table 3.

In some embodiments, in the case of heart failure, spinal injury, SMA and ALS, the Type V CRISPR system comprising chRDNA produces a genome modification that eliminates expression of genes that cause graft rejection. This genome modification enables implantation of exogenous neurons (in case of a spinal injury, SMA or ALS) or exogenous cardiomyocytes (in case of heart failure) without rejection of the exogenous neurons or cardiomyocytes by the recipient's immune system. In some embodiments, the invention is a method of treating a disease or condition of the central nervous system (CNS) comprising a gene modification effected by the Type V CRISPR system comprising chRDNA that results in the formation of HLA⁻/CIITA⁻ neurons, including motor neurons. In some embodiments, the invention is a method of treating heart failure comprising a gene modification effected by the Type V CRISPR system comprising chRDNA that results in the formation of HLA⁻/CIITA⁻ cardiomyocytes. In some embodiments, the Type V CRISPR system comprising chRDNA produces inactivation of one or more of MHC Class I genes selected from HLA-A, HLA-A2, HLA-B, HLA-C, HLA-E, HLA-F, and HLA-G, and further produces inactivation of the class II transactivator (CIITA) gene.

### Lipid Nanoparticles (LNP)

In some embodiments, the components of the Type V CRISPR system comprising chRDNA is encapsulated within a microscopic lipid droplet sometimes referred to as a lipid nanoparticle (LNP). Examples of LNPs are described e.g., in Sharma et al. (2014), Next generation delivery system for proteins and genes of therapeutic purpose: why and how? Biomed Res Int. 2014:327950 and further in the U.S. Patent Application Pub. Nos. US20190136231, Lipid nanoparticle formulations for CRISPR/Cas components; US20160317676, Methods and compositions for delivery of nucleic acids; US20190022247, Lipids and lipid nanoparticle compositions for delivery of nucleic acids; and US20210251898, Lipid nanoparticles for mRNA vaccines.

In some embodiments, the LNP used herein has a diameter of between about 100 nm and about 1 μm, preferably <100 nm. In some embodiments, the LNP includes one or more cationic lipids. The cationic lipids can be selected such that, when combined, the measured value of the pKₐ of the combination is no less than 6.1 and no greater than 6.7, e.g., between 6.2 and 6.6; or between 6.3 and 6.5. The cationic lipids can have a head group, one or more hydrophobic tails, and a linker between the head group and the one or more tails. The head group can include an amine which is a site of positive charge. The amine can be a primary, secondary, or tertiary amine, or a quaternary amine. The one or more hydrophobic tails can include two hydrophobic chains, which may be the same or different. The tails can be aliphatic chains, fatty acid chains or other hydrophobic chains. The linker can include, for example, a glyceride linker, an acyclic glyceride analog linker, or a cyclic linker. The linker can include functional groups such as an ether, an ester, a phosphate, a phosphonate, a phosphorothioate, a sulfonate, a disulfide, an acetal, a ketal, an imine, a hydrazone, or an oxime. Cationic lipids include one or more amine group(s) which bear the positive charge. Preferred cationic lipids are ionizable such that they can exist in a positively charged or neutral depending on pH. The ionization of the cationic lipid affects the surface charge of a lipid nanoparticle (LNP) and can influence plasma protein absorption, blood clearance, tissue distribution and the ability to fuse with cellular membranes.

In some embodiments, the LNP further comprises a neutral lipid. The neutral lipid can be selected from DSPC, DPPC, POPC, DOPE, or SM. The lipid capable of reducing aggregation can be a PEG lipid. In some embodiments, the lipid particle further includes a sterol. In some embodiments, the molar ratio of all cationic lipids in the particle is between about 20% and about 60%; the neutral lipid can be present in a molar ratio of about 5% to about 25%; the sterol can be present in a molar ratio of about 25% to about 55%; and the PEG lipid can be PEG-DMA, PEG-DMG, or a combination thereof, and can be present in a molar ratio of about 0.5% to about 15%.

Other examples of lipids used to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids include 98N12-5, C12-200, DLin-KC2-DMA (KC2), DLin-MC3-DMA (MC3), XTC, MD1, and 7C1. Other examples of PEG-modified lipids include PEG-CerC14, and PEG-CerC20.

In some embodiments, the LNP further comprises, a lipid capable of reducing aggregation, e.g., aggregation of LNPs.

In some embodiments, the surface of LNPs is further modified with polymers or lipids (e.g., chitosan, cationic polymers, or cationic lipids) or coupled to targeting molecules (antibodies specific for cell-surface receptors or natural ligands of cell surface receptors) to direct the nanoparticle to the appropriate cell type and increase the likelihood of cellular uptake as described e.g., in Jian et al., (2012) Cationic core shell liponanoparticles for ocular gene delivery, Biomaterials 33 (30): 7621-30).

### Delivery of LNPs

In some embodiments, the invention comprises delivery of the Type V CRISPR system comprising chRDNA described herein to the patient's cells and tissues. In some embodiments, the delivery is via a lipid nanoparticle (LNP) described herein. The delivery may be accomplished into patient's cells in vitro, ex vivo or in vivo. In some embodiments, the LNPs comprising the Type V CRISPR system comprising chRDNA are administered systemically to the patient (i.e., intravenously into systemic circulation). In some embodiments, the target nucleic acid is expressed (or aberrantly expressed) in a particular organ and the LNPs comprising the Type V CRISPR system comprising chRDNA are administered to that organ. In some embodiments, the LNPs comprising the Type V CRISPR system comprising chRDNA are contacted to the patient's cells ex vivo and the treated cells are administered to the patient.

In some embodiments, the target nucleic acid is expressed (or aberrantly expressed) in the liver. In such embodiments, the LNP comprising the Type V CRISPR system comprising chRDNA is administered systemically or into hepatic circulation (e.g., portal vein or another hepatic or liver-bound blood vessel).

In some embodiments, the target nucleic acid is expressed (or aberrantly expressed) in the liver and more specifically, in hepatocytes. In such embodiments, the patient's own (autologous) hepatocytes or donor (allogeneic) hepatocytes are treated ex vivo with the LNPs comprising the Type V CRISPR system comprising chRDNA and the treated hepatocytes are administered systemically to the patient or into hepatic circulation (e.g., portal vein or another hepatic or liver-bound blood vessel of the patient.

In some embodiments, the target nucleic acid is expressed (or aberrantly expressed) in sinusoidal endothelial cells of the liver or hematopoietic cells throughout the body (e.g., Factor VIII gene whose deficiency is the cause of hemophilia A). In such embodiments, the target cells for in vivo or ex vivo administration of the Type V CRISPR system comprising chRDNA include hepatic sinusoidal endothelial cells, progenitor cells which differentiate into hepatic sinusoidal endothelial cells, hematopoietic endothelial cells or progenitor cells which differentiate into hematopoietic endothelial cells.

In some embodiments, the target nucleic acid is expressed (or aberrantly expressed) in the cells of the eye. In some embodiments, the LNPs comprising the Type V CRISPR system comprising chRDNA are delivered into the eye (intraocular delivery). I some embodiments, the delivery is intravitreal. In some embodiments, the delivery is directly to the retina to reach the retinal pigment epithelium.

In some embodiments, administration is in combination with a pharmaceutically acceptable carrier.

Induced Pluripotent Stem Cells (iPSCs)

In some embodiments, the invention comprises a method of ex vivo cell-based therapy comprising editing the genome of induced pluripotent stem cells (iPSCs) using the Type V CRISPR system comprising chRDNA; differentiating the edited iPSC into a cell of desired lineage and implanting the differentiated cell into the patient.

In some embodiments, the iPSCs are patient-derived (autologous). In some embodiments somatic cells are obtained from a subject or patient, reprogrammed into induced pluripotent stem cell (iPSCs), genome-edited using the Type V CRISPR system comprising chRDNA as described herein, re-differentiated into cells of the desired cell type, and administered to the same subject or patient.

In some embodiments, the iPSCs are donor-derived or cell line-derived. In some embodiments, differentiation of iPSCs is artificially induced in vitro or ex vivo by administration of certain stimuli.

Reprogramming Cells into iPSC

In some embodiments, reprogramming of differentiated cells into iPSCs is artificially induced in vitro or ex vivo by administration of external agents. In some embodiments, reprogramming of differentiated cells into iPSCs comprises reversal of one or more heritable patterns of nucleic acid modification such as methylation. In some embodiments, reprogramming of differentiated cells into iPSCs is accomplished by expressing certain genes in the differentiated cells. In some embodiments, the genes are introduced into the cells using plasmid or viral expression vectors. In some embodiments, the genes are introduced as mRNA capable of being translated inside the cells. In some embodiments, the genes inducing the reprogramming are one or more of Oct4, Sox2, Klf4, and c-Myc as described in Takahashi et al., (2006) Induction of pluripotent stem cells from mouse embryonic and adult fibroblast cultures by defined factors, Cell 126 (4): 663-76. In some embodiments, the genes inducing the reprogramming are one or more of, or all three of Oct4, Sox2, and NANOG as described in Budniatzky et al., (2014) Concise review: reprogramming strategies for cardiovascular regenerative medicine: from induced pluripotent stem cells to direct reprogramming, Stem Cells Transl Med. 3 (4): 448-57 and references cited therein. In some embodiments, the genes inducing the reprogramming are one or more of Sox1, Sox3, Sox15, Sox18, Klf1, Klf2, Klf5, NR5A2, c-Myc, 1-Myc, n-Myc, Rem2, Tert, and LIN28 or Wnt. In some embodiments, reprogramming of somatic cells into iPSCs is enhanced by introducing one or more of a MEK inhibitor, a DNA methyltransferase inhibitor, a histone deacetylase (HDAC) inhibitor, valproic acid, 5′-azacytidine, dexamethasone, suberoylanilide, hydroxamic acid (SAHA), vitamin C, and trichostatin (TSA) Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylurcido) caproic hydroxamic acid), AOE (2-amino-8-oxo-9, 10-epoxydecanoic acid), CHAP31 and CHAP50.

In some embodiments, reprogramming of differentiated cells into iPSCs is assessed by detecting or measuring expression of markers associated with iPSCs. In some embodiments, the reprograming is assessed by detecting or measuring expression of one or more genes selected from SSEA3, SSEA4, CD9, Nanog, Fbx 15, Ecat1, Esg1, Eras, Gdf3, Fgf4, Cripto, Dax 1, Zpf296, Slc2a3, Rex1, Utf1, and Nat1. In some embodiments, reprograming into motor neurons is assessed by detecting or measuring expression of a combination of Sox1, Pax6, Nestin, HB9, MAP2, NeuroFilament, Tuj1 and Olig2. In some embodiments, the expression is assessed or measured by detecting the mRNA in question by Southern blotting or PCR, including reverse transcription PCR (RT-PCR) real time PCR (rtPCR) and digital droplet PCR (ddPCR), or nucleic acid sequencing. In some embodiments, the expression is confirmed by detecting the protein in question by immunological methods selected from Western blots, flow cytometry, immunochemistry and immunocytochemistry.

Modifying iPSCs

In some embodiments, the invention comprises a method of genetically modifying iPSCs in order to effect expression of a gene where aberrant expression of the gene is associated with a disease or condition in a patient. The genetically modified iPSCs are then differentiated into a cell type characterized by aberrant expression of the gene in a patient and the differentiated cells are administered to the patient to alleviate the symptoms of the disease or condition.

In some embodiments, iPSCs are cultured in a suitable medium (e.g., mTeSR-plus medium (STEMCELL Technologies, Cambridge, Mass.) with one or more supplements prior to nucleofection with Type V CRISPR system comprising chRDNA. In some embodiments, prior to nucleofection the cells are dispersed with accutase (STEMCELL Technologies, Cambridge, Mass.) In some embodiments, the cells are counted in order to achieve the desired number of cells in a nucleofection well. In some embodiments, 4×10³-2×10⁴ cells are present in a well of a 96-well plate. The Cas12a guide/nucleoprotein complex is added and the nucleofection is performed according to the manufacturer's recommendations. In some embodiments, the Nucleocuvette™ Plate and the Nucleofector™ instrument are used (Lonza, Allendale, NJ).

Differentiating iPSCs

In some embodiments, the method comprises a step of differentiating the iPSCs genetically modified using the Type V CRISPR system comprising chRDNA according to the method of the invention. In some embodiments, the iPSCs are differentiated into cells of the central nervous system (CNS) such as neurons, including motor neurons, retinal cells or glial cells, cells of the cardiovascular system such as endothelial cells or cardiac myocytes. In some embodiments, the iPSCs are differentiated into hepatic cells, or mesenchymal stem cells. In some embodiments, iPSCs are differentiated into ocular (non-neural) cells such as corneal, scleral or choroid cells.

In some embodiments, the iPSCs are differentiated into neurons. In some embodiments, the method includes the steps of preparing fresh cultures of confluent iPSCs and dissociating the confluent cultures of iPSCs prior to plating in neural induction medium comprising one or more of serum replacement, non-essential amino acids, glutamine or glutamine alternative, vitamins, GSK-3 inhibitors, TGF-beta receptor, or TGF-beta inhibitors, ALK inhibitors, dorsomorphin and compound E. In some embodiments, one or more exchanges of medium are performed. In some embodiments, the subsequent medium comprises growth factors such as FGF and EGF. In some embodiments, for differentiation and generation of motor neurons, the subsequent medium is MN induction medium and Neurobasal Medium. In some embodiments, the medium is further supplemented with one or more of all-trans-retinoic acid, Sonic Hedgehog protein, purmorphamine, SAG dihydrochloride, CNTF, and GDNF. In some embodiments, to assess differentiation into neurons, the cells assessed by fluorescent microscopy. In some embodiments, the cells are fixed in formaldehyde or paraformaldehyde, permeabilized, e.g., with Triton-X and/or Tween-20 and stained with primary antibodies capable of specific binding to one or more of Sox 1, Pax6, Nestin, HB9, MAP2, NeuroFilament, Tuj1 and Olig2. To further assess differentiation into motor neurons, calcium activity is assessed e.g., by imaging using Oregon Green 488 BAPTA-2 calcium indicator. In some embodiments, electrical activity of the cells is measured e.g., using the MultiClamp 700B Microelectrode Amplifier (Molecular Devices, San Jose, Cal.)

In some embodiments, the iPSCs are differentiated into myocytes, such as cardiomyocytes. In some embodiments, the iPSCs are freshly grown to achieve 60-70% confluency of the culture and treated with one or more of a GSK-3 inhibitor, and a Wnt-dependent phosphorylation blocker, and incubated in a medium containing insulin. In some embodiments, the cells are assessed by measuring gene expression of cardiomyocyte-specific markers (e.g., TBX5, TNNT2, MYH6 and MYL7), and optionally, the decrease in expression of pluripotency markers (NANOG, POUF5F1).

### Xenotransplantation

In some embodiments, the invention is a method of making transgenic non-human mammals for xenotransplantation by modifying the animal's genome using the Type V CRISPR system comprising chRDNA in order to eliminate expression of one or more genes that create immunological incompatibility and/or introduce one or more genes that establish immunological compatibility between a non-human donor and a human recipient, or decrease the likelihood of rejection by the human immune system.

In some embodiments, the transgenic non-human mammal is a pig, and the method comprises delivering the Type V CRISPR system comprising chRDNA into a porcine oocyte, ovum or zygote followed by transfer of the genetically modified oocyte, ovum or zygote into a foster female.

In some embodiments, the transgenic non-human mammal is a pig, and the method comprises delivering the Type V CRISPR system comprising chRDNA into a porcine somatic cell, and further comprises transferring the nucleus of the somatic cell into an enucleated ovum or zygote followed by transfer of the resulting ovum or zygote into a foster female.

In some embodiments, the genome modification is insertion of a functional copy of a gene that results in production of an exogenous protein. In some embodiments, the exogenous protein has cytoprotective properties, anticoagulant properties, complement inhibitor properties, or immunosuppressive properties. In some embodiments, the exogenous gene is a human gene. In some embodiments, the exogenous protein has cytoprotective properties, and the gene is selected from A20, HO-1, FAT-1, and TNF-alpha receptor. In some embodiments, the exogenous protein has anticoagulant properties, and the gene is selected from CD39, hirudin, TFPI, EPCR, and TBM. In some embodiments, the exogenous protein has complement inhibitor properties, and the gene is selected from CD46, DAF (CD55), CD59, and CR1. In some embodiments, the exogenous protein has immunosuppressive properties, and the gene is selected from CTLA4 and CD47.

In some embodiments, the genome modification is disruption of an endogenous gene that results in reduction or elimination of expression of an endogenous protein. In some embodiments, the disrupted endogenous gene is a part of the porcine Major Histocompatibility Complex (SLA complex) and is selected from SLA-1, SLA-2, SLA-3, SLA-6, SLA-7, SLA-8, SLA-9, SLA-11, and SLA-12. In some embodiments, disruption of one or more of porcine Class I SLA genes is accompanied by insertion of one or more of the human Class I HLA genes selected from HLA-A, HLA-A2, HLA-B, HLA-C, HLA-E, HLA-F, and HLA-G.

In some embodiments, the disrupted endogenous gene is one or both of alpha (1,3)-galactosyltransferase (GT), and cytidine monophosphate-N-acetylneuraminic acid hydroxylase (CMAH). Reducing or eliminating expression of GT and CMAH reduces immunogenicity of porcine xenotransplants by disrupting glycosylation of surface proteins in pig cells. GT catalyzes addition of galactose-alpha-1,3-galactose residues to glycoproteins. CMAH catalyzes conversion of N-acetylneuraminic acid (Neu5Ac) to N-glycolylneuraminic acid (Neu5Gc) which with involvement of other porcine enzymes, forms immunogenic glycoproteins found on the surface of pig cells but not found in human cells.

### Examples

### Example 1. Cloning, Expression, Production, and Assembly of Cas12a-Guide Nucleoprotein Complexes

A non-limiting example of successful preparation of a functional Cas12a-guide nucleoprotein complex is described in the International Patent application no. PCT/US2021/055394 filed on Oct. 18, 2021.

Briefly, the Acidaminococcus spp. (strain BV3L6) catalytically active Cas12a protein (AsCas12a) sequence can be codon optimized for expression in E. coli cells and conjugated to a nuclear localization sequence (NLS) via a linker, e.g., a glycine-serine linker. Several NLS sequences have been validated for Cas12a including nucleoplasmin (NLP) NLS and SV40 large T-antigen NLS. The DNA sequences encoding NLS-Cas9 may be cloned into suitable bacterial expression vectors using standard cloning methods.

The AsCas12a protein can be expressed in E. coli using an expression vector and purified using affinity chromatography, ion exchange, and size exclusion chromatography, essentially as described in, for example, Swarts et al. (Molecular Cell, 2017, 66:221-233).

Cas12a guides may be produced by linking a targeting region to a particular Cas12a guide activating region. A targeting region, or spacer, preferably comprised a 20-nucleotide target binding sequence. The target binding sequence was complementary to a target sequence that occurred downstream (in a 3′ direction) of a 5′-TTTV or 5′-TTTN PAM. Cas 12a guides (such as crRNAs and chRDNA) can be synthesized by commercial manufacturers or produced by in vitro transcription (e.g., T7 Quick High Yield RNA Synthesis Kit; New England Biolabs, Ipswich, Mass.)

Nucleoprotein complexes can be formed for example, at a concentration of 80 pmol Cas12a protein: 240 pmol guide. The Cas12a protein and each of the guide components (e.g., crRNA or chRDNA) can be adjusted to the desired total concentration, incubated for 2 minutes at 95° C., removed from a thermocycler, and allowed to equilibrate to room temperature. The Cas 12a protein was diluted to an appropriate concentration in binding buffer (60 mM TRIS-acetate, 150 mM potassium acetate, 30 mM magnesium acetate, at pH 7.9) to a final volume of 1.5 μl and mixed with the 1 μl of the guide components, followed by incubation at 37° C. for 10 minutes.

### Example 2. Tiling Target Genes for Cas12a Cleavage

For targeting a selected gene, all 20-nucleotide sequences downstream (in a 3′ direction) of a PAM motif (e.g., 5′-TTTV) can be used for targeting. Target selection criteria included, but are not limited to, homology to other regions in the genome; percent G-C content; melting temperature; and presence of homopolymer within the spacer.

The identified 20-nucleotide sequences can be appended downstream (in a 3′ direction) to the AsCas12a guide activating region sequence and the desired guides can be produced.

Optionally, the guides can be designed to include one or more deoxyribonucleotides (DNA) among the ribonucleotides (RNA) in the targeting region or the activating region.

Further optionally, the guides can be designed to include one or more chemically modified nucleotides. Chemical modifications can include backbone modifications, nitrogenous base modifications and sugar modifications. One example is a phosphorothioate modification of the nucleic acid backbone.

### Example 3. (Prophetic) Transfection of Human Induced Pluripotent Stem Cells With Cas12a-chRDNA Nucleoprotein Complexes

This Example describes the nucleofection of iPSCs with a Cas12a guide/nucleoprotein complex. Cas12a (AsCas12a) is prepared as described herein. Guides are prepared for the target sequences of genes listed in Table 3.

The cells are prepared as follows. The iPSCs are cultured in mTeSR-plus medium (STEMCELL Technologies, Cambridge, Mass.), supplemented with Rho-associated, coiled-coil-containing protein kinase inhibitor (“ROCKi,” MilliporeSigma, Burlington, Mass.) at a final concentration of 10 uM for 3 hours at 37° C. prior to transfection. The mTeSR-plus/ROCKi media is removed and the iPSCs are washed with 10 mL of PBS, followed by the addition of 3 mL of accutase (STEMCELL Technologies, Cambridge, Mass.) and the cells are incubated for 5-10 minutes at 37° C. 7 mL of mTeSR-pulse and ROCKi is then added to the cells, and the cells are mixed and counted. Cells are then centrifuged, the medium removed, and the cells are washed with 10 mL of PBS, centrifuged again and PBS removed. The cells are resuspended in Nucleofector™ P4 or P3 (Lonza, Allendale, NJ) solution to a density of 2×10⁵-10⁶ cells/ml per sample. 20 μl of the cell suspension was then added to each well containing 2.5 μl of the Cas12a guide/nucleoprotein complexes, and the entire volume from each well was transferred to a well of a 96-well Nucleocuvette™ Plate (Lonza, Allendale, NJ). The plate was loaded onto the Nucleofector™ 96-well Shuttle and cells nucleofected using the CA137 Nucleofector™ program. Post-nucleofection, 77.5 μl of ImmunoCult-XF complete medium supplemented with IL-2 (100 units/mL) was added to each well, and the entire volume of transfected cell suspension was transferred to a 96-well cell culture plate containing 100 μl pre-warmed ImmunoCult-XF complete medium supplemented with IL-2 (100 units/mL). The plate was transferred to a tissue culture incubator and maintained at 37° C. in 5% CO₂ for 48 hours before downstream analysis.

### Example 4. (Prophetic) Differentiation of Nucleofected iPSCs into Motor Neurons

This example describes differentiation of nucleofected iPSCs into motor neurons. Briefly, the protocol from Bianchi, F., et al., ((2018) Rapid and efficient differentiation of functional motor neurons from iPSC for neural injury, Stem Cell Research 32:126) is used. After nucleofection, the iPSCs are allowed to rest and grown to confluence. Confluent iPSCs are dissociated using accutase and plated at 0.5×10⁶ cells/well on MG-coated 6-well plates in neural induction medium (NIM), consisting of a 1:1 mix of KO-DMEM/F: 12 and neurobasal medium (NBM) supplemented with 10% KnockOut Serum Replacement, 1% Non-Essential Amino Acids (NEAA) (all from ThermoFisher Scientific), and 1% GlutaMAX, 0.1 mML-ascorbic acid, 3 uM CHIR99021 (both from Sigma Aldrich), 2 uM SB431542 (CellGuidance Systems), 1 μM dorsomorphin and 1 μM compound E (both from StemCell). 1% RevitaCell (ThermoFisher Scientific) is added for the first 24 h only. NIM is replaced daily for six days, after which cells were dissociated with accutase, and plated in NPC expansion medium, consisting of a 1:1 mix of KO-DMEM: F12 and NBM, supplemented with 1% P/S, 1% B27, 1% N2, 1% NEAA, 1% GlutaMAX, 0.1 mML-AA, 10 ng/mL bFGF and 10 ng/mL EGF. Optionally, one fifth of the cells are retained at each passage for marker analysis.

For differentiation and generation of motor neurons, NPCs are cultured for 6 days in MN induction medium, consisting of a 1:1 mix of KO-DMEM: F12 and Neurobasal Medium supplemented with 1% P/S, 1% B27, 1% N2, 1% Non-Essential Amino Acids, 1% GlutaMAX, 0.1 mML-ascorbic acid, 10 uM all-trans retinoic acid, 100 ng/ml recombinant SHH, 1 μM Purmorphamine (Abcam) and 1 mM SAG Dihydrochloride (Sigma Aldrich). After seven days, cells are dissociated using accutase, and re-plated in maturation medium, consisting of 1:1 KO-DMEM: F12 and NBM, supplemented with 1% P/S, 1% B27, 1% N2, 1% NEAA, 1% GlutaMAX, 0.1 mML-AA, 10 ng/mL CNTF, 10 ng/ml BDNF, 10 ng/mL NT-3 and 10 ng/ml GDNF.

### Cell Analysis

To assess differentiation, the cells are fixed in 3.75% paraformaldehyde solution in phosphate buffered saline (PBS), blocked and permeabilized with 0.1% Triton-X, 0.1% Tween-20 and 2.5% BSA in PBS. Primary antibodies (e.g., from AbCam) to Sox1, Pax6, Nestin, HB9, MAP2, NeuroFilament, Tuj1 and Olig2 are added. Next, cells are counter-stained with AlexaFluor-labeled secondary antibodies. Cell nuclei were labelled e.g., with NucBlue. Cells are imaged using a fluorescence microscope, e.g., an inverted fluorescence microscope.

To further assess differentiation into motor neurons, calcium activity is imaged using Oregon Green 488 BAPTA-2 calcium indicator. Cells are incubated in a dye solution in an imaging medium, e.g., FluoroBrite-DMEM Imaging Medium for 30 min, washed twice in PBS, and further incubated in fresh FBDMEM for 30 min. Cells were imaged using standard FITC filters, and fluorescence intensities from individual segmented cells is recorded in time. To further assess differentiation into motor neurons, electrical activity of the cells is measured using the MultiClamp 700B Microelectrode Amplifier (Molecular Devices, San Jose, Cal.)

### Example 5 (Prophetic) Differentiation of Nucleofected iPSCs into Cardiomyocytes

This example describes differentiation of nucleofected iPSCs into cardiomyocytes. Briefly, the protocol from Balafkan, N., et al., ((2020) A method for differentiating human induced pluripotent stem cells toward functional cardiomyocytes in 96-well microplates, Nature 10:18498) is used. After nucleofection, the iPSCs are allowed to rest and grown to confluence. Advanced DMEM/F-12 with Geltrex (both Thermo Fisher Scientific) are used to coat wells in the 96-well plate. Before plating, the human iPSC colonies are converted into a homogeneous cell suspension and plated at a density of 2.4×10⁴ cells/cm² using Essential 8 Medium (Thermo Fisher Scientific) and incubated for 3 days with daily changes of medium to achieve 60-70% confluency of the culture. Then cells are treated with a GSK-3 inhibitor (e.g., CHIR99021) in a medium (e.g., RPMI 1640). After 24 h the medium is changed to no GSK-3 inhibitor and cells are left for 48 h (days 1-2). On day 3 cells are treated with 5 uM Wnt-dependent phosphorylation blocker (e.g., IWP2) and incubated for further 48 hrs (days 3-4). On day 5 the medium is changed to no Wnt-P inhibitor and cells are left for 48 h (days 5-6). At day 7 medium is changed to contain insulin. Cells are assessed by measuring gene expression of cardiomyocyte specific markers (e.g., TBX5, TNNT2, MYH6 and MYL7), decrease in expression of pluripotency markers (NANOG, POUF5F1).

### Example 6. Culture of Immortalized Mouse Hepatocytes

This Example illustrates the culture of the immortalized mouse hepatocyte line H2.35 (ATCC; Manassas, VA).

H2.35 cells were retrieved from liquid nitrogen storage, and cells were thawed in a 37° C. water bath for 3 minutes. Cells are diluted into calcium and magnesium-free phosphate buffered saline (PBS; Thermo Scientific, Wilmington, DE) to a final volume of 10 mLs and are centrifuged at 300 g for 5 minutes. PBS is aspirated and cells are resuspended in 10 mL of prewarmed H2.35 medium comprising Dulbecco's Modified Eagle Medium (DMEM; Thermo Scientific, Wilmington, DE) with 1 g/mL glucose supplemented with 4% fetal bovine serum and 200 nM dexamethasone (Merck/Millipore-Sigma, Munich, Germany). The cells were counted using the Countess® 3 Automated Cell Counter (Life Technologies; Grand Island, NY). Cells were then cultured in an adherent flatbottom flask at a density of 10,000 cells/cm² and cultured at 32° C. in 10% CO₂.

Cells are routinely passaged when confluency reaches 60-70% by aspirating media and washing with sufficient PBS to cover the bottom of the flask and gently rocking back and forth. PBS is then aspirated, and room temperature (RT) Accutase (Thermo Scientific, Wilmington, DE) is added to sufficient volume to cover the bottom of the flask, followed by gently rocking the flask back and forth, and incubated at RT for 3 minutes. Flask was gently tapped against the palm of the hand five to ten times to loosen cells, and 2.5×volumes of H2.35 medium relative to the Accutase volume is added to the flask and mixed using a serologic pipette. Cells are centrifuged at 300 g for 5 minutes, medium decanted, and counted using a Countess® 3 Automated Cell Counter and seeded into a new flask at a density of 10,000-20,000 cells/cm² in H2.35 medium.

### Example 7. Cloning, Expression, Production, and Assembly of Cas12a Guide/Nucleoprotein Complexes

This Example describes a method for cloning, expressing, and purifying Cas12a guide/nucleoprotein complexes, as well as methods of producing Cas 12a guide components.

### A. Cloning of a Cas12 Protein

The Acidaminococcus spp. (strain BV3L6) catalytically active Cas 12a protein sequence (SEQ ID NO: 1) was codon optimized for expression in E. coli cells. At the C-terminus, a glycine-serine linker and one nuclear localization sequence (NLS) (SEQ ID NO: 2) was added. Oligonucleotide sequences coding for the Cas12a-NLS protein (referred to as the AsCas12a and Cas 12a protein in the following Examples) were provided to commercial manufacturers for synthesis. DNA sequences were then cloned into suitable bacterial expression vectors using standard cloning methods.

### B. Expression and Purification of a Cas12a Protein

The AsCas12a protein was expressed in E. coli using an expression vector and purified using affinity chromatography, ion exchange, and size exclusion chromatography, essentially as described in, for example, Swarts et al. (Molecular Cell, 2017, 66:221-233).

### C. Production of Cas12a Guide Components

Cas12a guides were produced by linking a targeting region to a particular Cas12a guide activating region. A targeting region, or spacer, preferably comprised a 20-nucleotide target binding sequence. The target binding sequence was complementary to a target sequence that occurred downstream (in a 3′ direction) of a 5′-TTTV or 5′-TTTN PAM. Exemplary Cas12a guide activating region sequences are SEQ ID NO: 6, SEQ ID NO: 8, and SEQ ID NO: 10, for the Acidaminococcus spp., L. bacterium, and F. novicida Cas12a species, respectively.

Cas 12a guide sequences (such as crRNAs and chRDNA) were provided to a commercial manufacturer for synthesis.

Guide RNA components (such as crRNAs) can be produced by in vitro transcription (e.g., T7 Quick High Yield RNA Synthesis Kit; New England Biolabs, Ipswich, MA) from double-stranded (ds) DNA templates by incorporating a T7 promoter at the 5′ end of the dsDNA template sequences.

### D. Assembly of a Cas12a Guide/Nucleoprotein Complex

Acidaminococcus spp. Cas12a (AsCas12a) tagged with a C-terminal nuclear localization sequence was recombinantly expressed in E. coli and purified using chromatographic methods. Nucleoprotein complexes were formed at a concentration of 80 pmol Cas12a protein: 240 pmol guide, unless otherwise stated. Prior to assembly with Cas12a protein, each of the guide components (e.g., crRNA or chRDNA) was adjusted to the desired total concentration (240 μmol) in a final volume of 1 μl, incubated for 2 minutes at 95° C., removed from a thermocycler, and allowed to equilibrate to room temperature. The Cas12a protein was diluted to an appropriate concentration in binding buffer (60 mM TRIS-acetate, 150 mM potassium acetate, 30 mM magnesium acetate, at pH 7.9) to a final volume of 1.5 μl and mixed with the 1 μl of the guide components, followed by incubation at 37° C. for 10 minutes. Cas12a guide/nucleoprotein complexes are used immediately, or were frozen down at −20° C. until needed.

### Example 8. Electroporation of Immortalized Mouse Hepatocytes with Cas12a Guide/Nucleoprotein Complexes

This Example illustrates the electroporation of the immortalized mouse hepatocyte line H2.35 with Cas12a guide/nucleoprotein complexes for gene editing.

The Cas12a guide/nucleoprotein complexes of Example 7 were transfected into H2.35 using the Nucleofector™ 96-well Shuttle System (Lonza, Allendale, NJ). The Cas12a guide/nucleoprotein complex were dispensed in a 2.5 μl final volume into individual wells of a 96-well plate. The H2.35 cells were removed from a culture flask in a manner similar to process described in Example 1. After counting of the H2.35, 100,000 cells were resuspended in 17.5 uL of CTS™ Xenon™ Electroporation Buffer (Thermo Scientific, Wilmington, DE) per transfection condition and mixed with the 2.5 uL of the Cas12a guide/nucleoprotein complex and transferred to a well of a 96-well Nucleocuvette™ Plate (Lonza, Allendale, NJ). The plate was loaded onto the Nucleofector™ 96-well Shuttle (Lonza, Allendale, NJ) and cells were nucleofected using the EH-110 Nucleofector™ program (Lonza, Allendale, NJ).

Post-nucleofection, 80 μl of H2.35 medium was added to each well, and the entire volume of transfected cell suspension was transferred to a 96-well cell culture plate containing 100 μl pre-warmed H2.35 medium. The plate was transferred to a tissue culture incubator and maintained at 32° C. in 10% CO₂ for 72 hours before downstream analysis.

### Example 9. Tiling of Mouse Genes with Cas12a Guide/Nucleoprotein Complexes

This Example describes the design and use of Cas 12a guide/nucleoprotein complexes to target the genes encoding the mouse Proprotein convertase subtilisin/kexin type 9 (PCSK9), Transthyretin (TTR), and Angiopoietin Like 3 (ANGPTL3) in the immortalized mouse hepatocyte line H2.35.

A. Designing the AsCas12a crRNA Guides

A collection of 20-nucleotide sequences downstream (in a 3′ direction) of a 5′-TTTV PAM motif in the coding regions of the genes encoding mouse PCSK9, TTR, and ANGPLT3, were selected for targeting (SEQ ID Nos: 5-94). Target selection criteria included, but were not limited to, homology to other regions in the genome; percent G-C content; melting temperature; and presence of homopolymer within the spacer.

The identified 20-nucleotide sequences were appended downstream (in a 3′ direction) to the AsCas12a activating region sequence (SEQ ID NO: 3)

Sequences were provided to commercial manufacturers for synthesis. Then, individual Cas12a guide/nucleoprotein complexes were prepared as described in Example 7 and transfected into primary H2.35 cells as described in Example 3.

### B. Determining Genome Editing Efficiency

(1) Target dsDNA sequence generation for deep sequencing gDNA was isolated from the nucleofected H2.35 cells 72 hours after transfection using the Cas12a guide/nucleoprotein complexes and 50 uL QuickExtract™ DNA extraction solution (Epicentre, Madison, WI) per well, followed by incubation at 37° C. for 10 minutes, 65° C. for 30 minutes, and 95° C. for 3 minutes to stop the reaction. The isolated gDNA was diluted with 50 μL sterile water and samples were stored at −20° C.

Using the isolated gDNA, a first PCR was performed using Q5 Hot Start High-Fidelity 2X Master Mix (New England Biolabs, Ipswich, MA) at 1× concentration, primers designed to amplify the region around the Cas 12a target were used at 0.5 uM each, and 3.75 μL of gDNA was used in a final volume of 10 uL. Amplification was conducted by an initial cycle at 98° C. for 1 minute, 35 cycles of 10s at 98° C., and 20 seconds at 60° C., 30 seconds at 72° C.; and a final extension at 72° C. for 2 minutes. The PCR reactions were diluted 1:100 in water.

A unique set of index primers for a barcoding PCR were used to facilitate multiplex sequencing for each sample. Barcoding PCRs were performed using a reaction mix comprising Q5 Hot Start High-Fidelity 2X Master Mix (New England Biolabs, Ipswich, MA) at 1× concentration, primers at 0.5 μM each, and 1 μL of 1:100 diluted first PCR in a final volume of 10 μL. The reaction mixtures were amplified as follows: 98° C. for 1 minute; followed by 12 cycles of 10s at 98° C., 20 seconds at 60° C., and 30 seconds at 72° C.; with a final extension reaction at 72° C. for 2 minutes.

### (2) SPRIselect Clean-Up

The PCR reactions were pooled and transferred into a single microfuge tube for SPRIselect (Beckman Coulter, Pasadena, CA) bead-based cleanup of amplicons for sequencing.

To the amplicon, 0.9× volumes of SPRIselect beads were added, mixed, and incubated at room temperature for 10 minutes. The microfuge tube was placed on a magnetic tube stand until the solution cleared. Supernatant was removed and discarded, the residual beads were washed with 1 volume of 85% ethanol, and the beads were incubated at room temperature for 30 seconds. After incubation, ethanol was aspirated, and the beads were air-dried at room temperature for 10 minutes. The microfuge tube was removed from the magnetic stand and 0.25× volumes of Qiagen EB buffer (Qiagen, Venlo, Netherlands) was added to the beads, mixed vigorously, and incubated for 2 minutes at room temperature. The microfuge tube was returned to the magnet, incubated until the solution had cleared, and supernatant containing the purified amplicons was dispensed into a clean microfuge tube. The purified amplicons were quantified using the Nanodrop™ 2000 System (Thermo Scientific, Wilmington, DE) and library quality analyzed using the Fragment Analyzer™ System (Advanced Analytical Technologies, Ames, IA) and the DNF-910 dsDNA Reagent Kit (Advanced Analytical Technologies, Ames, IA).

### (3) Deep Sequencing Set-Up

The pooled amplicons were normalized to a 4 nM concentration as calculated from the Nanodrop™ 2000 System values and the average size of the amplicons. The library was analyzed on a MiSeq Sequencer (Illumina, San Diego, CA) with MiSeq Reagent Kit v2 (Illumina, San Diego, CA) for 300 cycles with two 151-cycle paired-end runs and two 8-cycle index reads.

### (4) Deep Sequencing Data Analysis

The identities of products in the sequencing data were determined based on the index barcode sequences adapted onto the amplicons in the barcoding PCR. A computational script was used to process the MiSeq data that executes, for example, the following tasks:
a. Reads were aligned to the mouse genome (build GRCm38/mm10) using Bowtie (bowtie-bio.sourceforge.net/index.shtml) software;
b. Aligned reads were compared to the expected wild type genomic locus sequence, and reads not aligning to any part of the wild type locus discarded;
c. Reads matching wild type sequence were tallied;
d. Reads with indels (insertion or deletion of bases) were categorized by indel type and tallied; and
e. Total indel reads were divided by the sum of wild type reads and indel reads to give percent-mutated reads.

Through the identification of indel sequences at regions targeted by the Cas12a guide/nucleoprotein complexes, the resulting genome editing efficiency of the Cas12a guide/nucleoprotein complexes was determined. The results of the in-cell editing experiment are shown in Table 7 below.

TABLE 7
Percent Indels Detected with Cas12a
crRNA/nucleoprotein Complexes

| SEQ ID NO: | Target ID | % editing | StDev |
|---|---|---|---|
| SEQ ID NO: 05 | mAGP-tgt1 | 4.6% | 1.1% |
| SEQ ID NO: 06 | mAGP-tgt2 | 7.8% | 1.2% |
| SEQ ID NO: 07 | mAGP-tgt3 | 85.6% | 1.2% |
| SEQ ID NO: 08 | mAGP-tgt4 | 0.2% | 0.1% |
| SEQ ID NO: 09 | mAGP-tgt5 | 19.2% | 0.2% |
| SEQ ID NO: 10 | mAGP-tgt6 | 74.1% | 3.6% |
| SEQ ID NO: 11 | mAGP-tgt7 | 49.5% | 3.6% |
| SEQ ID NO: 12 | mAGP-tgt8 | 87.7% | 3.0% |
| SEQ ID NO: 13 | mAGP-tgt9 | 5.8% | 0.0% |
| SEQ ID NO: 14 | mAGP-tgt10 | 26.7% | 4.9% |
| SEQ ID NO: 15 | mAGP-tgt11 | 85.6% | 1.8% |
| SEQ ID NO: 16 | mAGP-tgt12 | 12.6% | 1.8% |
| SEQ ID NO: 17 | mAGP-tgt13 | 1.5% | 0.1% |
| SEQ ID NO: 18 | mAGP-tgt14 | 53.0% | 3.5% |
| SEQ ID NO: 19 | mAGP-tgt15 | 0.6% | 0.0% |
| SEQ ID NO: 20 | mAGP-tgt16 | 37.2% | 7.6% |
| SEQ ID NO: 21 | mAGP-tgt17 | 1.1% | 0.2% |
| SEQ ID NO: 22 | mAGP-tgt18 | 88.5% | 5.6% |
| SEQ ID NO: 23 | mAGP-tgt19 | 22.4% | 5.1% |
| SEQ ID NO: 24 | mAGP-tgt20 | 86.2% | 2.8% |
| SEQ ID NO: 25 | mAGP-tgt21 | 20.6% | 3.3% |
| SEQ ID NO: 26 | mAGP-tgt22 | 30.6% | 3.0% |
| SEQ ID NO: 27 | mAGP-tgt23 | 8.1% | 2.1% |
| SEQ ID NO: 28 | mAGP-tgt24 | 13.1% | 1.6% |
| SEQ ID NO: 29 | mAGP-tgt25 | 75.8% | 5.4% |
| SEQ ID NO: 30 | mAGP-tgt26 | 16.3% | 2.8% |
| SEQ ID NO: 31 | mAGP-tgt27 | 1.7% | 0.3% |
| SEQ ID NO: 32 | mAGP-tgt28 | 0.7% | 0.1% |
| SEQ ID NO: 33 | mAGP-tgt29 | 10.3% | 1.5% |
| SEQ ID NO: 34 | mAGP-tgt30 | 75.9% | 3.9% |
| SEQ ID NO: 35 | mAGP-tgt31 | 37.3% | 2.8% |
| SEQ ID NO: 36 | mAGP-tgt32 | 5.2% | 1.5% |
| SEQ ID NO: 37 | mAGP-tgt33 | 21.7% | 2.8% |
| SEQ ID NO: 38 | mAGP-tgt34 | 3.6% | 0.4% |
| SEQ ID NO: 39 | mAGP-tgt35 | 13.4% | 3.3% |
| SEQ ID NO: 40 | mAGP-tgt36 | 77.4% | 6.1% |
| SEQ ID NO: 41 | mAGP-tgt37 | 15.5% | 2.2% |
| SEQ ID NO: 42 | mAGP-tgt38 | 54.9% | 38.7% |
| SEQ ID NO: 43 | mAGP-tgt39 | 47.8% | 5.2% |
| SEQ ID NO: 44 | mAGP-tgt40 | 52.3% | 5.6% |
| SEQ ID NO: 45 | mAGP-tgt41 | 5.4% | 0.9% |
| SEQ ID NO: 46 | mAGP-tgt42 | 62.6% | 3.7% |
| SEQ ID NO: 47 | mAGP-tgt43 | 21.2% | 4.9% |
| SEQ ID NO: 48 | mAGP-tgt44 | 2.1% | 0.3% |
| SEQ ID NO: 49 | mAGP-tgt45 | 70.6% | 4.5% |
| SEQ ID NO: 50 | mAGP-tgt46 | 62.3% | 5.6% |
| SEQ ID NO: 51 | mAGP-tgt47 | nd | nd |
| SEQ ID NO: 52 | mAGP-tgt48 | nd | nd |
| SEQ ID NO: 53 | mAGP-tgt49 | nd | nd |
| SEQ ID NO: 54 | mAGP-tgt50 | nd | nd |
| SEQ ID NO: 55 | mAGP-tgt51 | nd | nd |
| SEQ ID NO: 56 | mAGP-tgt52 | 78.0% | 10.3% |
| SEQ ID NO: 57 | mAGP-tgt53 | 32.5% | 6.8% |
| SEQ ID NO: 58 | mAGP-tgt54 | 19.4% | 4.8% |
| SEQ ID NO: 59 | mAGP-tgt55 | 30.6% | 9.6% |
| SEQ ID NO: 60 | mAGP-tgt56 | 4.6% | 1.5% |
| SEQ ID NO: 61 | mAGP-tgt57 | 65.5% | 9.0% |
| SEQ ID NO: 62 | mAGP-tgt58 | 91.2% | 6.1% |
| SEQ ID NO: 63 | mAGP-tgt59 | 58.9% | 1.1% |
| SEQ ID NO: 64 | mAGP-tgt60 | 33.7% | 1.3% |
| SEQ ID NO: 65 | mAGP-tgt61 | 0.8% | 0.1% |
| SEQ ID NO: 66 | mAGP-tgt62 | 32.3% | 10.5% |
| SEQ ID NO: 67 | mAGP-tgt63 | 10.6% | 3.6% |
| SEQ ID NO: 68 | mAGP-tgt64 | 27.4% | 1.9% |
| SEQ ID NO: 69 | mAGP-tgt65 | 16.3% | 2.0% |
| SEQ ID NO: 70 | mPCK9-tgt1 | 3.4% | 0.9% |
| SEQ ID NO: 71 | mPCK9-tgt2 | 54.7% | 8.1% |
| SEQ ID NO: 72 | mPCK9-tgt3 | 5.2% | 1.7% |
| SEQ ID NO: 73 | mPCK9-tgt4 | 17.2% | 2.0% |
| SEQ ID NO: 74 | mPCK9-tgt5 | 70.3% | 7.1% |
| SEQ ID NO: 75 | mPCK9-tgt6 | 66.5% | 6.1% |
| SEQ ID NO: 76 | mPCK9-tgt7 | 5.0% | 1.0% |
| SEQ ID NO: 77 | mPCK9-tgt8 | 20.8% | 5.9% |
| SEQ ID NO: 78 | mPCK9-tgt9 | 55.1% | 2.9% |
| SEQ ID NO: 79 | mPCK9-tgt10 | 44.0% | 3.4% |
| SEQ ID NO: 80 | mPCK9-tgt11 | 9.8% | 0.5% |
| SEQ ID NO: 81 | mPCK9-tgt12 | nd | nd |
| SEQ ID NO: 82 | mPCK9-tgt13 | 27.7% | 2.6% |
| SEQ ID NO: 83 | mPCK9-tgt14 | 1.1% | 0.1% |
| SEQ ID NO: 84 | mTR-tgt1 | 43.1% | 3.7% |
| SEQ ID NO: 85 | mTR-tgt2 | 1.9% | 0.5% |
| SEQ ID NO: 86 | mTR-tgt3 | 2.3% | 0.2% |
| SEQ ID NO: 87 | mTR-tgt4 | 6.1% | 0.6% |
| SEQ ID NO: 88 | mTR-tgt5 | nd | nd |
| SEQ ID NO: 89 | mTR-tgt6 | 0.2% | 0.0% |
| SEQ ID NO: 90 | mTR-tgt7 | 0.5% | 0.0% |
| SEQ ID NO: 91 | mTR-tgt8 | 27.3% | 2.8% |
| SEQ ID NO: 92 | mTR-tgt9 | 90.9% | 2.0% |
| SEQ ID NO: 93 | mTR-tgt10 | 5.6% | 1.8% |
| SEQ ID NO: 94 | mTR-tgt11 | 6.4% | 0.6% |

StDev = standard deviation;
nd = not determined
n = 3

The data presented in Table 7 above demonstrate that Cas12a crRNA/nucleoprotein complexes are capable of on-target editing multiple genes in mouse H2.35 cells. Other genes, such as those described elsewhere herein, can be targeted in a similar manner, using AsCas12a or other Cas12a proteins (such as L. bacterium or F. novicida).

### Example 10. Engineering Cas12a chRDNA Guide Molecules with DNA in the Activating Region Sequence

The following Example describes the engineering of AsCas12a chRDNA guide molecules to comprise DNA bases in the activating region sequence.

A. In Silico Cas 12a chRDNA Guide Design

The 20-nucleotide activating region sequence (SEQ ID NO:03) of the AsCas12a guide was selected for engineering and DNA bases were designed in place of RNA at positions 1, 3, 7, 10, 12, 14, 15, and 19 (counting from a 5′ to 3′ direction along the guide).

Nine target sequences were selected from the list of targets shown in Example 9 Table 7 and engineered with DNA bases in the activating region sequence, as well as a Cas12a crRNA control sequence, and were provided to a commercial manufacturer for synthesis.

### B. Cell Transfection and Analysis

Individual Cas12a guide/nucleoprotein complexes for screening were prepared essentially as described in Example 7. The nucleoprotein complexes were transfected into H2.35 cells as described in Example 8, and the resulting genome editing efficiency of the Cas12a guide/nucleoprotein complexes was determined as described in Example 9. The results of the in-cell editing experiment are shown in Table 8 below.

TABLE 8
AsCas12a chRDNA Guide with DNA in Activating Region Sequence

| | Target | DNA | % | |
|---|---|---|---|---|
| SEQ ID NO: | ID | position | Editing | StDev |
| SEQ ID NO: 148 | mPCK9-tgt5_crRNA | — | 71.6% | 8.0% |
| SEQ ID NO: 149 | mPCK9-tgt5_V3′ | 1, 3, 7, 10, 12, | 77.5% | 2.8% |
| | | 14, 15, 19 | | |
| SEQ ID NO: 150 | mPCK9-tgt6_crRNA | — | 85.3% | 1.1% |
| SEQ ID NO: 151 | mPCK9-tgt6_V3′ | 1, 3, 7, 10, 12, | 83.2% | 2.0% |
| | | 14, 15, 19 | | |
| SEQ ID NO: 152 | mTR-tgt9_crRNA | — | 97.1% | 0.3% |
| SEQ ID NO: 153 | mTR-tgt9_V3′ | 1, 3, 7, 10, 12, | 94.8% | 0.3% |
| | | 14, 15, 19 | | |
| SEQ ID NO: 154 | mAGP-tgt58_crRNA | — | 98.3% | 0.3% |
| SEQ ID NO: 155 | mAGP-tgt58_V3′ | 1, 3, 7, 10, 12, | 96.6% | 0.9% |
| | | 14, 15, 19 | | |
| SEQ ID NO: 156 | mAGP-tgt18_crRNA | — | 95.7% | 0.6% |
| SEQ ID NO: 157 | mAGP-tgt18_V3′ | 1, 3, 7, 10, 12, | 94.7% | 0.6% |
| | | 14, 15, 19 | | |
| SEQ ID NO: 158 | mAGP-tgt8_crRNA | — | 96.5% | 0.3% |
| SEQ ID NO: 159 | mAGP-tgt8_V3′ | 1, 3, 7, 10, 12, | 95.2% | 0.8% |
| | | 14, 15, 19 | | |
| SEQ ID NO: 160 | mAGP-tgt20_crRNA | — | 88.4% | 1.4% |
| SEQ ID NO: 161 | mAGP-tgt20_V3′ | 1, 3, 7, 10, 12, | 75.8% | 1.0% |
| | | 14, 15, 19 | | |
| SEQ ID NO: 162 | mAGP-tgt3_crRNA | — | 95.6% | 0.8% |
| SEQ ID NO: 163 | mAGP-tgt3_V3′ | 1, 3, 7, 10, 12, | 94.1% | 1.8% |
| | | 14, 15, 19 | | |
| SEQ ID NO: 164 | mAGP-tgt11_crRNA | — | 92.5% | 2.6% |
| SEQ ID NO: 165 | mAGP-tgt11_V3′ | 1, 3, 7, 10, 12, | 91.1% | 2.6% |
| | | 14, 15, 19 | | |

StDev = standard deviation;
n = 3

The data presented in Table 8 above demonstrate that AsCas12a guide with DNA in the activating region sequence are capable of on-target editing rates equivalent to all-RNA guides (compare, for example, SEQ ID NO: 148 to SEQ ID NO: 149; SEQ ID NO: 153 to SEQ ID NO: 154; SEQ ID NO: 160 to SEQ ID NO: 161). Other guides, such as those described elsewhere herein, can be engineered with DNA in the activating region sequence in a similar manner, using AsCas12a or other Cas 12a proteins (such as L. bacterium or F. novicida).

### Example 11. Engineering Cas12a chRDNA Guide Molecules with DNA in the Guide Repeat Sequence

The following Example describes the engineering of AsCas12a chRDNA guide molecules to comprise DNA bases in the target binding sequence.

A. In Silico Cas12a chRDNA Guide Design

One target sequence in the genes encoding mouse PCSK9 (PCSK9-tgt9), TTR (TTR-tgt5), and ANGPTL3 (ANGPLT3-tgt18) were selected for engineering. A Cas12a chRDNA guide for each target comprising an individual DNA base at a subset of positions in the target binding sequence, as well as a Cas12a crRNA control sequence, were provided to a commercial manufacturer for synthesis (SEQ ID NO. 74, SEQ ID NO. 92, SEQ ID NO. 22, and SEQ ID NO. 95-130).

### B. Cell Transfection and Analysis

Individual Cas12a guide/nucleoprotein complexes for screening were prepared essentially as described in Example 7. The nucleoprotein complexes were transfected into H2.35 cells as described in Example 8, and the resulting genome editing efficiency of the Cas12a guide/nucleoprotein complexes was determined as described in Example 9. The results of the in-cell editing experiment are shown in Table 9 below.

TABLE 9
Editing Rates of Cas12a Guide/nucleoprotein Complexes
Comprising DNA in the Target Binding Sequence

| | Target | DNA | % | |
|---|---|---|---|---|
| SEQ ID NO: | ID | position | Editing | StDev |
| SEQ ID NO: 148 | mPCK9-tgt5 | — | 91.0% | 1.2% |
| SEQ ID NO: 95 | mPCK9-tgt5_d1 | 21 | 64.7% | 6.0% |
| SEQ ID NO: 96 | mPCK9-tgt5_d8 | 28 | 43.2% | 3.2% |
| SEQ ID NO: 97 | mPCK9-tgt5_d9 | 29 | 56.8% | 3.7% |
| SEQ ID NO: 98 | mPCK9-tgt5_d10 | 30 | 56.1% | 1.7% |
| SEQ ID NO: 99 | mPCK9-tgt5_d11 | 31 | 56.5% | 6.3% |
| SEQ ID NO: 100 | mPCK9-tgt5_d12 | 32 | 45.8% | 3.1% |
| SEQ ID NO: 101 | mPCK9-tgt5_d14 | 34 | 58.3% | 4.7% |
| SEQ ID NO: 102 | mPCK9-tgt5_d15 | 35 | 59.7% | 2.4% |
| SEQ ID NO: 103 | mPCK9-tgt5_d17 | 37 | 65.1% | 2.6% |
| SEQ ID NO: 104 | mPCK9-tgt5_d18 | 38 | 50.5% | 3.5% |
| SEQ ID NO: 105 | mPCK9-tgt5_d19 | 39 | 65.2% | 1.2% |
| SEQ ID NO: 106 | mPCK9-tgt5_d20 | 40 | 73.6% | 2.0% |
| SEQ ID NO: 152 | mTR-tgt9 | — | 95.9% | 1.7% |
| SEQ ID NO: 107 | mTR-tgt9_d1 | 21 | 83.5% | 1.4% |
| SEQ ID NO: 108 | mTR-tgt9_d8 | 28 | 67.9% | 20.5% |
| SEQ ID NO: 109 | mTR-tgt9_d9 | 29 | 84.9% | 1.9% |
| SEQ ID NO: 110 | mTR-tgt9_d10 | 30 | 81.0% | 3.6% |
| SEQ ID NO: 111 | mTR-tgt9_d11 | 31 | 80.0% | 3.1% |
| SEQ ID NO: 112 | mTR-tgt9_d12 | 32 | 64.4% | 3.6% |
| SEQ ID NO: 113 | mTR-tgt9_d14 | 34 | 79.1% | 2.0% |
| SEQ ID NO: 114 | mTR-tgt9_d15 | 35 | 72.8% | 2.0% |
| SEQ ID NO: 115 | mTR-tgt9_d17 | 37 | 79.8% | 3.0% |
| SEQ ID NO: 116 | mTR-tgt9_d18 | 38 | 67.3% | 4.8% |
| SEQ ID NO: 117 | mTR-tgt9_d19 | 39 | 84.8% | 2.5% |
| SEQ ID NO: 118 | mTR-tgt9_d20 | 40 | 85.9% | 3.4% |
| SEQ ID NO: 156 | mAGP-tgt18 | — | 95.2% | 0.2% |
| SEQ ID NO: 119 | mAGP-tgt18_d1 | 21 | 78.7% | 1.3% |
| SEQ ID NO: 120 | mAGP-tgt18_d8 | 28 | 78.7% | 1.9% |
| SEQ ID NO: 121 | mAGP-tgt18_d9 | 29 | 70.3% | 13.2% |
| SEQ ID NO: 122 | mAGP-tgt18_d10 | 30 | 74.9% | 2.6% |
| SEQ ID NO: 123 | mAGP-tgt18_d11 | 31 | 70.8% | 12.9% |
| SEQ ID NO: 124 | mAGP-tgt18_d12 | 32 | 76.0% | 2.4% |
| SEQ ID NO: 125 | mAGP-tgt18_d14 | 34 | 77.0% | 2.6% |
| SEQ ID NO: 126 | mAGP-tgt18_d15 | 35 | 80.9% | 1.8% |
| SEQ ID NO: 127 | mAGP-tgt18_d17 | 37 | 78.6% | 4.1% |
| SEQ ID NO: 128 | mAGP-tgt18_d18 | 38 | 76.6% | 0.5% |
| SEQ ID NO: 129 | mAGP-tgt18_d19 | 39 | 80.2% | 3.8% |
| SEQ ID NO: 130 | mAGP-tgt18_d20 | 40 | 82.1% | 2.5% |

StDev = standard deviation;
n = 3

The editing results in Table 9 above demonstrate that Cas 12a chRDNA guide molecules comprising DNA in the spacer are capable of editing at a rate comparable to the crRNA across multiple targets (compare SEQ ID NO: 148 to SEQ ID NO: 103; SEQ ID NO: 152 to SEQ ID NO: 109; SEQ ID NO: 156 to SEQ ID NO: 130). The editing rates of the chRDNA guide designs in Table 3 were normalized to the editing rates of the crRNA for each target and were used to determine which positions within the target binding sequence of a the selected targets can be engineering as DNA in the Cas12a guide.

### Example 12. Cas12a chRDNA Guide Molecules with Multiple DNA Bases in the Target Binding Sequence

This Example describes the designing and testing of Cas12a chRDNA guide molecules with multiple DNA bases in the target binding sequence.

A. In Silico Design of Cas12a chRDNA Guides

The 20-nucleotide sequence of three targets in the gene encoding human B2M (B2M-tgt12, B2M-tgt1, B2M-intron-tgt12), a target in the gene encoding human TRAC (TRAC-tgt12), and a target in the gene encoding human DNA methyltransferase 1 (DNMT1-tgt1), were selected for editing. For each target, between 1 and 7 nucleotides of DNA were designed into the target binding sequence of each AsCas12a guide. Design criteria for the position of DNA bases included, but were not limited to, previously single position screen data (see Example 10), prior consensus of positions tolerant to DNA, distance between individual DNA bases in target binding sequence, and known location of mismatches in an off-target sequence. Cas12a chRDNA guide designs, as well as a control sequence with no DNA in the target binding sequence (“V3′” in Table 10), were provided to a commercial manufacturer for synthesis.

### B. Cell Transfection and Analysis

Individual Cas12a guide/nucleoprotein complexes for screening were prepared essentially as described in Example 7. The Cas12a guide/nucleoprotein complexes were transfected into primary T cells as described in Example 8, and the resulting genome editing efficiency of the Cas12a guide/nucleoprotein complexes was determined as described in Example 9. The results of the in-cell editing experiment, and the location of DNA bases in the target binding sequence of each Cas12a chRDNA guide, are shown in Table 10 below.

TABLE 10
Editing Rates of Cas12a Guide/nucleoprotein Complexes
Comprising DNA in the Target Sequence

| SEQ ID NO: | Target ID | DNA position | % Editing | StDev |
|---|---|---|---|---|
| SEQ ID NO: 131 | mPCK9-tgt5_d1.20. | 21 and 40 | 68.61% | 9.1% |
| SEQ ID NO: 132 | mPCK9-tgt5_d1.17.20. | 21, 37, and 40 | 61.04% | 7.6% |
| SEQ ID NO: 133 | mPCK9-tgt5_d1.17. | 21 and 37 | 42.58% | 20.2% |
| SEQ ID NO: 134 | mPCK9-tgt5_d17.20. | 37 and 40 | 66.50% | 8.6% |
| SEQ ID NO: 135 | mPCK9-tgt5_d7.16. | 27 and 36 | 0.90% | 0.4% |
| SEQ ID NO: 136 | mPCK9-tgt5_V3′ | — | 65.39% | 6.4% |
| SEQ ID NO: 137 | mTR-tgt9_d1.9.20. | 21, 29, and 40 | 86.96% | 3.4% |
| SEQ ID NO: 138 | mTR-tgt9_d9.19.20. | 21, 39, 40 | 85.23% | 5.7% |
| SEQ ID NO: 139 | mTR-tgt9_d1.9.19. | 21, 29, and 39 | 87.53% | 3.2% |
| SEQ ID NO: 140 | mTR-tgt9_d1.9.19.20. | 21, 29, 39, and 40 | 86.28% | 3.6% |
| SEQ ID NO: 141 | mTR-tgt9_V3′ | — | 54.90% | 9.6% |
| SEQ ID NO: 142 | mAGP-tgt18_d1.15.20. | 21, 35, and 40 | 85.37% | 2.4% |
| SEQ ID NO: 143 | mAGP-tgt18_d1.8.15. | 21, 28, and 35 | 68.76% | 6.5% |
| SEQ ID NO: 144 | mAGP-tgt18_d8.15.20. | 28, 35, and 40 | 42.83% | 23.9% |
| SEQ ID NO: 145 | mAGP-tgt18_d1.8.19. | 21, 28, and 39 | 69.02% | 13.9% |
| SEQ ID NO: 146 | mAGP-tgt18_d15.17.20. | 35, 37, and 40 | 80.76% | 3.1% |
| SEQ ID NO: 147 | mAGP-tgt18_V3′ | — | 85.78% | 5.8% |

StDev = standard deviation;
n = 3

The editing results in Table 10 above demonstrate that Cas12a chRDNA guide molecules comprising multiple DNA bases in the target binding sequence are capable of editing at a rate comparable to the crRNA across multiple targets (compare SEQ ID NO: 136 to SEQ ID NO: 131; SEQ ID NO: 141 to SEQ ID NO: 139, or SEQ ID NO: 147 to SEQ ID NO: 142).

While the invention has been described in detail with reference to specific examples, it will be apparent to one skilled in the art that various modifications can be made within the scope of this invention. Thus, the scope of the invention should not be limited by the examples described herein, but by the claims presented below.

What is claimed is:

1-11. (canceled)

12. A therapeutic composition for treating a disease or condition characterized by aberrant expression of a gene, the composition comprising:
(a) a first nucleoprotein complex comprising a Cas 12a protein and a first CRISPR guide molecule having a targeting region capable of binding a first target nucleic acid sequence; and an activating region capable of forming a nucleoprotein complex with the Cas 12a protein, and the Cas12a protein is capable of cleaving the first target nucleic acid wherein said first CRISPR guide molecule comprises at least one deoxyribonucleotide; and
(b) a donor polynucleotide comprising a coding sequence of the gene target aberrantly expressed in individuals suffering from the disease or condition;
wherein the first nucleoprotein complex and the donor polynucleotide are present in a lipid nanoparticle, and wherein the gene target is selected from Table 3.

13. The composition of claim 12, wherein in the CRISPR guide molecule, the activating region, the targeting region, or both comprise at least one deoxyribonucleotide.

14. (canceled)

15. (canceled)

16. The composition of claim 12, wherein the lipid nanoparticle comprises one or more cationic lipids with pKₐ of the lipid or combination of two or more lipids is between 6.1 and 6.7.

17. The composition of claim 12, wherein the lipid nanoparticle comprises a neutral lipid.

18. The composition of claim 12, wherein the lipid nanoparticle comprises a sterol.

19. The composition of claim 12, wherein the lipid nanoparticle comprises one or more lipids selected from the group consisting of DSPC, DPPC, POPC, DOPE, SM, PEG-DMA, PEG-DMG, DOTMA, DOSPA, DOTAP, DMRIE, DC-cholesterol, DOTAP-cholesterol, GAP-DMORIE-DPyPE, GL67A-DOPE-DMPE-PEG, 98N12-5, C12-200, DLin-KC2-DMA (KC2), DLin-MC3-DMA (MC3), XTC, MD1, 7C1, PEG-CerC14, and PEG-CerC20.

20. The composition of claim 12, further comprising a pharmaceutically acceptable carrier.

21-90. (canceled)

91. A method of making a transgenic animal for xenotransplantation, the method comprising:
(1) introducing into a cell of an animal:
a first nucleoprotein complex comprising a Cas12a protein and a first CRISPR guide molecule having a targeting region capable of binding a first target nucleic acid sequence; and an activating region capable of forming a nucleoprotein complex with the Cas 12a protein, and the Cas12a protein is capable of cleaving the first target nucleic acid wherein said first CRISPR guide molecule comprises at least one deoxyribonucleotide;
wherein cleavage by the Cas 12a protein results in a modification of a gene target selected from Table 6;
(2) introducing the cell into a foster female animal.

92. The method of claim 91, wherein the cell of an animal is an oocyte, ovum, or zygote.

93. The method of claim 91, wherein the cell of an animal is a somatic cell and the method further comprises after step (1), transferring the nucleus of the cell into an enucleated ovum or zygote.

94. The method of claim 91, wherein the animal is a pig.

95. The method of claim 91, wherein in the CRISPR guide molecule, the activating region, the targeting region, or both comprise at least one deoxyribonucleotide.

96. (canceled)

97. (canceled)

98. The method of claim 91, further comprising introducing into the cell a donor polynucleotide comprising a coding sequence of the gene target selected from A20, HO-1, FAT-1, TNF-alpha receptor, CD39, hirudin, TFPI, EPCR, TBM, CD46, DAF (CD55), CD59, CR1, CTLA4, CD47, one or more of Class I HLA.

99. The method of claim 98, wherein the cleavage with the Cas12a protein results in an insertion of the coding sequence into the genome of the cell and an increased expression of the gene in the cell.

100. (canceled)

101. The method of claim 91, wherein the cleavage with the Cas12a protein results in a disruption in the genome of the cell of a coding sequence of a gene target selected from GGTA1, b4GalNT2, CMAH, GT (alpha (1,3)-galactosyltransferase), GHR, one or more of Class I SLA.

102. (canceled)

103. A composition for making a transgenic animal for xenotransplantation, comprising an animal cell comprising:
i. a first nucleoprotein complex comprising a Cas 12a protein and a first CRISPR guide molecule having a targeting region capable of binding a first target nucleic acid sequence; and an activating region capable of forming a nucleoprotein complex with the Cas12a protein, and the Cas12a protein is capable of cleaving the first target nucleic acid wherein said first CRISPR guide molecule comprises at least one deoxyribonucleotide;
wherein cleavage by the Cas 12a protein results in a modification of a gene target selected from Table 6.

104. (canceled)

105. (canceled)

106. (canceled)

107. The composition of claim 103, wherein in the CRISPR guide molecule, the activating region, the targeting region, or both comprise at least one deoxyribonucleotide.

108. (canceled)

109. (canceled)

110. The composition of claim 103, further comprising a donor polynucleotide comprising a coding sequence of the gene target selected from A20, HO-1, FAT-1, TNF-alpha receptor, CD39, hirudin, TFPI, EPCR, TBM, CD46, DAF (CD55), CD59, CR1, CTLA4, CD47, one or more of Class I HLA.

111-114. (canceled)

115. A method of treating a disease or condition characterized by aberrant expression of a gene, the method comprising introducing into a somatic cell of a patient suffering from a disease or condition the composition of claim 12.
